SEQUENCE LISTING (24 sequences)

SEQ ID NO: 1 - protein, 181 aa, Homo sapiens
MESKEPQLKGIVTRLFSQQGYFLQMHPDGTIDGTKDENSDYTLFNLIPVGLRVVAIQGVKASLYVAMNGEGYLYSSDVFTPECKFKESVFENYYVIYSSTLYRQQESGRAWFLGLNKEGQIMKGNRVKKTKPSSHFVPKPIEVCMYREQSLHEIGEKQGRSRKSSGTPTMNGGKVVNQDST

SEQ ID NO: 2 - DNA, 2754 bp, Homo sapiens
aaatctgctgtgcatccagagagcaaagtgggatgatctgtcactacacctgcagcaccacgctcggaggacagctcctgcctgcagcttccagacccaggaagcctgaggggaaggaaggaagtacgggcgaaatcatcagattggcttcccagatttgggaatctgaagcgggcccacatcttccggccaacttccattgaacttcccagcactcgaaagggaccgaaatggagagcaaagaaccccagctaaaagggattgtgacaaggttattcagccagcagggatacttcctgcagatgcacccagatggtaccattgatgggaccaaggacgaaaacagcgactacactctcttcaatctaattcccgtgggcctgcgtgtagtggccatccaaggagtgaaggctagcctctatgtggccatgaatggtgaaggctatctctacagttcagatgttttcactccagaatgcaaattcaaggaatctgtgtttgaaaactactatgtgatctattcttccacactgtaccgccagcaagaatcaggccgagcttggtttctgggactcaataaagaaggtcaaattatgaaggggaacagagtgaagaaaaccaagccctcatcacattttgtaccgaaacctattgaagtgtgtatgtacagagaacaatcgctacatgaaattggagaaaaacaagggcgttcaaggaaaagttctggaacaccaaccatgaatggaggcaaagttgtgaatcaagattcaacatagctgagaactctccccttcttccctctctcatcccttccccttcccttccttcccatttacccatttccttccagtaaatccacccaaggagaggaaaataaaatgacaacgcaagacctagtggctaagattctgcactcaaaatcttcctttgtgtaggacaagaaaattgaaccaaagcttgcttgttgcaatgtggtagaaaattcacgtgcacaaagattagcacacttaaaagcaaaggaaaaaataaatcagaactcaataaatattaaactaaactgtattgttattagtagaaggctaattgtaatgaagacattaataaagatgaaataaacttattactttaaaggaaaggatttggagaattgaactcacaaactgatgttatatactcaatagcttaaactcatgataatgctgcgatgtgtggttttgcttgattttgtattttatttgggcatctggaattgacacaccattacattctgtttgcaggattttttttgtaaccatgaaattgaacatttccaaattataaactatgttaatacctataaaatatatagccaggaaccatttatcatcaagaaaagtgtaagaaattatttttgagatgtaatttaagattgttttatgtaaaaggaaaatcttgtatggcatcgaatagccttaatgaatttaattctttcacaaaaatgatttcaaattatcctagagtataacatttttatcaaagatattatttccggagttcttctttctttcttttttttttttttttagtaatttagcaaaaacattactgttctaatgctgaagtgacttttgccagtgccatgtccaggtggtgaggtataagttacttgctcttagcatttggtctgatttttttgctttgtggacacctttgagagtatccacaaagcaatgtctcaggtgtggacacctgagagcatgttttagaaagctttgtaccctgtcttgtggcaggaaagaaagaacaggggttttacataaggaaataagtcctaggaaattagtcaacgcaaattgcatttgcctttgtaccttaccacagtcttatattgttttttaaactctgccatgaaatttggagacatgactgtgaaattcctaacttactatcttacaaagccagtagctaatttgttgctctatgtatgatcctgttacaagtccagtttgcaattcatttgtttcctagaacacagaagggtaccagtaatacactaaatgttcaaggtgtgtagagaaataatatggaattagcagctatgactccaacagacaggattgtgtgagcagctgaaaggagcaaaaaagaactcagtgtaagagaaggcacatacatagttaagaatactaaagtatttttaaaaatcaaggaagaaataaatgttacacaatttgcattggaataaatagatctatttagtcctacaaatcaggagtggtgtagagacatccaaatttaaagaaaaaaaaacacaaaacagaatgttaaaaatgtatgcagatttatggatattatcaatgagaagacatagcatgtaacttctcctatatctctactgtccagcatgtattgttccaaatatgactccctaaaatatatacactttgcagaagctctaggccctcacctcaaaccttgccattggttgccgtatttcaaggtcaatatagtttccctcactttacacaatcattattcttcaatagtggaccatatccttcaccaggtatcctatttctgttatctagaggttagcagaaaatgaaatgaaggaatttccctaagcagttgggaagaacaaattgtatgcatgtaggcaaagattttgaagatacatttgcaagagatatttgtttaaccaaaatatttggaaagtaacaaataaagacatttaaattttctaaaaaaaaaaaaaaaaacaaaaaaaaaaaaaaa

SEQ ID NO: 3 - protein, 399 aa, Homo Sapiens
MRPERPRPRGSAPGPMETPPWDPARNDSLPPTLTPAVPPYVKLGLTVVYTVFYALLFVFIYVQLWLVLRYRHKRLSYQSVFLFLCLFWASLRTVLFSFYFKDFVAANSLSPFVFWLLYCFPVCLQFFTLTLMNLYFTQVIFKAKSKYSPELLKYRLPLYLASLFISLVFLLVNLTCAVLVKTGNWERKVIVSVRVAINDTLFVLCAVSLSICLYKISKMSLANIYLESKGSSVCQVTAIGVTVILLYTSRACYNLFILSFSQNKSVHSFDYDWYNVSDQADLKNQLGDAGYVLFGVVLFVWELLPTTLVVYFFRVRNPTKDLTNPGMVPSHGFSPRSYFFDNPRRYDSDDDLAWNIAPQGLQGGFAPDYYDWGQQTNSFLAQAGTLQDSTLDPDKPSLG

SEQ ID NO: 4 - DNA, 1705 bp, Homo sapiens
gcggcttgttttctttcctccagtctcggggctgcaggctgagcgcgatgcgcggagacccccgcgggggcggcggcggccgtgagccccgatgaggcccgagcgtccccggccgcgcggcagcgcccccggcccgatggagaccccgccgtgggacccagcccgcaacgactcgctgccgcccacgctgaccccggccgtgcccccctacgtgaagcttggcctcaccgtcgtctacaccgtgttctacgcgctgctcttcgtgttcatctacgtgcagctctggctggtgctgcgttaccgccacaagcggctcagctaccagagcgtcttcctctttctctgcctcttctgggcctccctgcggaccgtcctcttctccttctacttcaaagacttcgtggcggccaattcgctcagccccttcgtcttctggctgctctactgcttccctgtgtgcctgcagtttttcaccctcacgctgatgaacttgtacttcacgcaggtgattttcaaagccaagtcaaaatattctccagaattactcaaataccggttgcccctctacctggcctccctcttcatcagccttgttttcctgttggtgaatttaacctgtgctgtgctggtaaagacgggaaattgggagaggaaggttatcgtctctgtgcgagtggccattaatgacacgctcttcgtgctgtgtgccgtctctctctccatctgtctctacaaaatctctaagatgtccttagccaacatttacttggagtccaagggctcctccgtgtgtcaagtgactgccatcggtgtcaccgtgatactgctttacacctctcgggcctgctacaacctgttcatcctgtcattttctcagaacaagagcgtccattcctttgattatgactggtacaatgtatcagaccaggcagatttgaagaatcagctgggagatgctggatacgtattatttggagtggtgttatttgtttgggaactcttacctaccaccttagtcgtttatttcttccgagttagaaatcctacaaaggaccttaccaaccctggaatggtccccagccatggattcagtcccagatcttatttctttgacaaccctcgaagatatgacagtgatgatgaccttgcctggaacattgcccctcagggacttcagggaggttttgctccagattactatgattggggacaacaaactaacagcttcctggcacaagcaggaactttgcaagactcaactttggatcctgacaaaccaagccttgggtagcatcagttaacagttttatggacgattcctcagatgaaaagcttcagaaaagcatagtgacagctgaatttttagggcacttttccttaagaaatagaacttgatttttatttgttacaggtttccaatggccccataggaataagcaataatgtagactgataaacccttattttagtactaaagagggagccttgctatttcagtgggtataatttaaactttttaaagaaaatctgtacttttataaagatgtattttgtataacttaaataataatgctaaagtatactagggtttttttttcttgagaatgttactgcaatcatgttgtagtttgcacagacttttatgcataattcactttaaaaatatagaatatatggtctaatagttaaaaaaaaaaaaaaaaaa

SEQ ID NO: 5 - protein, 540 aa, Homo sapiens
MARKQNRNSKELGLVPLTDDTSHARPPGPGRALLECDHLRSGVPGGRRRKDWSCSLLVASLAGAFGSSFLYGYNLSVVNAPTPYIKAFYNESWERRHGRPIDPDTLTLLWSVTVSIFAIGGLVGTLIVKMIGKVLGRKHTLLANNGFAISAALLMACSLQAGAFEMLIVGRFIMGIDGGVALSVLPMYLSEISPKEIRGSLGQVTAIFICIGVFTGQLLGLPELLGKESTWPYLFGVIVVPAVVQLLSLPFLPDSPRYLLLEKHNEARAVKAFQTFLGKADVSQEVEEVLAESRVQRSIRLVSVLELLRAPYVRWQVVTVIVTMACYQLCGLNAIWFYTNSIFGKAGIPLAKIPYVTLSTGGIETLAAVFSGLVIEHLGRRPLLIGGFGLMGLFFGTLTITLTLQDHAPWVPYLSIVGILAIIASFCSGPGGIPFILTGEFFQQSQRPAAFIIAGTVNWLSNFAVGLLFPFIQKSLDTYCFLVFATICITGAIYLYFVLPETKNRTYAEISQAFSKRNKAYPPEEKIDSAVTDGKINGRP

SEQ ID NO: 6 - DNA, 1863 bp, Homo sapiens
cttggcagagtctggggtccctggactgagccatcagctgggtcactgagacccatggcaaggaaacaaaataggaattccaaggaactgggcctagttcccctcacagatgacaccagccacgccaggcctccagggccagggagggcactgctggagtgtgaccacctgaggagtggggtgccaggtggaaggagaagaaaggactggtcctgctcgctcctcgtggcctccctcgcgggcgccttcggctcctccttcctctacggctacaacctgtcggtggtgaatgcccccaccccgtacatcaaggccttttacaatgagtcatgggaaagaaggcatggacgtccaatagacccagacactctgactctgctctggtctgtgactgtgtccatattcgccatcggtggacttgtggggacattaattgtgaagatgattggaaaggttcttgggaggaagcacactttgctggccaataatgggtttgcaatttctgctgcattgctgatggcctgctcgctccaggcaggagcctttgaaatgctcatcgtgggacgcttcatcatgggcatagatggaggcgtcgccctcagtgtgctccccatgtacctcagtgagatctcacccaaggagatccgtggctctctggggcaggtgactgccatctttatctgcattggcgtgttcactgggcagcttctgggcctgcccgagctgctgggaaaggagagtacctggccatacctgtttggagtgattgtggtccctgccgttgtccagctgctgagccttccctttctcccggacagcccacgctacctgctcttggagaagcacaacgaggcaagagctgtgaaagccttccaaacgttcttgggtaaagcagacgtttcccaagaggtagaggaggtcctggctgagagccgcgtgcagaggagcatccgcctggtgtccgtgctggagctgctgagagctccctacgtccgctggcaggtggtcaccgtgattgtcaccatggcctgctaccagctctgtggcctcaatgcaatttggttctataccaacagcatctttggaaaagctgggatccctctggcaaagatcccatacgtcaccttgagtacagggggcatcgagactttggctgccgtcttctctggtttggtcattgagcacctgggacggagacccctcctcattggtggctttgggctcatgggcctcttctttgggaccctcaccatcacgctgaccctgcaggaccacgccccctgggtcccctacctgagtatcgtgggcattctggccatcatcgcctctttctgcagtgggccaggtggcatcccgttcatcttgactggtgagttcttccagcaatctcagcggccggctgccttcatcattgcaggcaccgtcaactggctctccaactttgctgttgggctcctcttcccattcattcagaaaagtctggacacctactgtttcctagtctttgctacaatttgtatcacaggtgctatctacctgtattttgtgctgcctgagaccaaaaacagaacctatgcagaaatcagccaggcattttccaaaaggaacaaagcatacccaccagaagagaaaatcgactcagctgtcactgatggtaagataaatggaaggccttaacaagtttcctcctccacgttggacaattatgtcaaaaacaggattgtctacatggatgatctcacttttcaggaaacttaaaatttacccattattgggaagcttaaatgaattgaagctatgcaagtcttttatattattaaatatttaaaagtaaacctgtactaatctaaaaaaaaaaaaaaa

SEQ ID NO: 7 - protein, 2248 aa, Homo sapiens
MAHNAGAAAAAGTHSAKSGGSEAALKEGGSAAALSSSSSSSAAAAAASSSSSSGPGSAMETGLLPNHKLKTVGEAPAAPPHQQHHHHHHAHHHHHHAHHLHHHHALQQQLNQFQQQQQQQQQQQQQQQQQQHPISNNNSLGGAGGGAPQPGPDMEQPQHGGAKDSAAGGQADPPGPPLLSKPGDEDDAPPKMGEPAGGRYEHPGLGALGTQQPPVAVPGGGGGPAAVPEFNNYYGSAAPASGGPGGRAGPCFDQHGGQQSPGMGMMHSASAAAAGAPGSMDPLQNSHEGYPNSQCNHYPGYSRPGAGGGGGGGGGGGGGSGGGGGGGGAGAGGAGAGAVAAAAAAAAAAAGGGGGGGYGGSSAGYGVLSSPRQQGGGMMMGPGGGGAASLSKAAAGSAAGGFQRFAGQNQHPSGATPTLNQLLTSPSPMMRSYGGSYPEYSSPSAPPPPPSQPQSQAAAAGAAAGGQQAAAGMGLGKDMGAQYAAASPAWAAAQQRSHPAMSPGTPGPTMGRSQGSPMDPMVMKRPQLYGMGSNPHSQPQQSSPYPGGSYGPPGPQRYPIGIQGRTPGAMAGMQYPQQQDSGDATWKETFWLMPPQYGQQGVSGYCQQGQQPYYSQQPQPPHLPPQAQYLPSQSQQRYQPQQDMSQEGYGTRSQPPLAPGKPNHEDLNLIQQERPSSLPDLSGSIDDLPTGTEATLSSAVSASGSTSSQGDQSNPAQSPFSPHASPHLSSIPGGPSPSPVGSPVGSNQSRSGPISPASIPGSQMPPQPPGSQSESSSHPALSQSPMPQERGFMAGTQRNPQMAQYGPQQTGPSMSPHPSPGGQMHAGISSFQQSNSSGTYGPQMSQYGPQGNYSRPPAYSGVPSASYSGPGPGMGISANNQMHGQGPSQPCGAVPLGRMPSAGMQNRPFPGNMSSMTPSSPGMSQQGGPGMGPPMPTVNRKAQEAAAAVMQAAANSAQSRQGSFPGMNQSGLMASSSPYSQPMNNSSSLMNTQAPPYSMAPAMVNSSAASVGLADMMSPGESKLPLPLKADGKEEGTPQPESKSKKSSSSTTTGEKITKVYELGNEPERKLWVDRYLTFMEERGSPVSSLPAVGKKPLDLFRLYVCVKEIGGLAQVNKNKKWRELATNLNVGTSSSAASSLKKQYIQYLFAFECKIERGEEPPPEVFSTGDTKKQPKLQPPSPANSGSLQGPQTPQSTGSNSMAEVPGDLKPPTPASTPHGQMTPMQGGRSSTISVHDPFSDVSDSSFPKRNSMTPNAPYQQGMSMPDVMGRMPYEPNKDPFGGMRKVPGSSEPFMTQGQMPNSSMQDMYNQSPSGAMSNLGMGQRQQFPYGASYDRRHEPYGQQYPGQGPPSGQPPYGGHQPGLYPQQPNYKRHMDGMYGPPAKRHEGDMYNMQYSSQQQEMYNQYGGSYSGPDRRPIQGQYPYPYSRERMQGPGQIQTHGIPPQMMGGPLQSSSSEGPQQNMWAARNDMPYPYQNRQGPGGPTQAPPYPGMNRTDDMMVPDQRINHESQWPSHVSQRQPYMSSSASMQPITRPPQPSYQTPPSLPNHISRAPSPASFQRSLENRMSPSKSPFLPSMKMQKVMPTVPTSQVTGPPPQPPPIRREITFPPGSVEASQPVLKQRRKITSKDIVTPEAWRVMMSLKSGLLAESTWALDTINILLYDDSTVATFNLSQLSGFLELLVEYFRKCLIDIFGILMEYEVGDPSQKALDHNAARKDDSQSLADDSGKEEEDAECIDDDEEDEEDEEEDSEKTESDEKSSIALTAPDAAADPKEKPKQASKFDKLPIKIVKKNNLFVVDRSDKLGRVQEFNSGLLHWQLGGGDTTEHIQTHFESKMEIPPRRPPPPLSSAGRKKEQEGKGDSEEQQEKSIIATIDDVLSARPGALPEDANPGPQTESSKFPFGIQQAKSHRNIKLLEDEPRSRDETPLCTIAHWQDSLAKRCICVSNIVRSLSFVPGNDAEMSKHPGLVLILGKLILLHHEHPERKRAPQTYEKEEDEDKGVACSKDEWWWDCLEVLRDNTLVTLANISGQLDLSAYTESICLPILDGLLHWMVCPSAEAQDPFPTVGPNSVLSPQRLVLETLCKLSIQDNNVDLILATPPFSRQEKFYATLVRYVGDRKNPVCREMSMALLSNLAQGDALAARAIAVQKGSIGNLISFLEDGVTMAQYQQSQHNLMHMQPPPLEPPSVDMMCRAAKALLAMARVDENRSEFLLHEGRLLDISISAVLNSLVASVICDVLFQIGQL

SEQ ID NO: 8 - DNA, 9648 bp, Homo sapiens
atggcccataacgcgggcgccgcggccgccgccggcacccacagcgccaagagcggcggctccgaggcggctctcaaggagggtggaagcgccgccgcgctgtcctcctcctcctcctcctccgcggcggcagcggcggcatcctcttcctcctcgtcgggcccgggctcggccatggagacggggctgctccccaaccacaaactgaaaaccgttggcgaagcccccgccgcgccgccccaccagcagcaccaccaccaccaccatgcccaccaccaccaccaccatgcccaccacctccaccaccaccacgcactacagcagcagctaaaccagttccagcagcagcagcagcagcagcaacagcagcagcagcagcagcagcaacagcaacatcccatttccaacaacaacagcttgggcggcgcgggcggcggcgcgcctcagcccggccccgacatggagcagccgcaacatggaggcgccaaggacagtgctgcgggcggccaggccgaccccccgggcccgccgctgctgagcaagccgggcgacgaggacgacgcgccgcccaagatgggggagccggcgggcggccgctacgagcacccgggcttgggcgccctgggcacgcagcagccgccggtcgccgtgcccgggggcggcggcggcccggcggccgtcccggagtttaataattactatggcagcgctgcccctgcgagcggcggccccggcggccgcgctgggccttgctttgatcaacatggcggacaacaaagccccgggatggggatgatgcactccgcctccgccgccgccgccggggcccccggcagcatggaccccctgcagaactcccacgaagggtaccccaacagccagtgcaaccattatccgggctacagccggcccggcgcgggcggcggcggcggcggcggcggcggaggaggaggaggcagcggaggaggaggaggaggaggaggagcaggagcaggaggagcaggagcgggagctgtggcggcggcggccgcggcggcggcggcagcagcaggaggcggcggcggcggcggctatgggggctcgtccgcggggtacggggtgctgagctccccccggcagcagggcggcggcatgatgatgggccccgggggcggcggggccgcgagcctcagcaaggcggccgccggctcggcggcggggggcttccagcgcttcgccggccagaaccagcacccgtcgggggccaccccgaccctcaatcagctgctcacctcgcccagccccatgatgcggagctacggcggcagctaccccgagtacagcagccccagcgcgccgccgccgccgccgtcgcagccccagtcccaggcggcggcggcgggggcggcggcgggcggccagcaggcggccgcgggcatgggcttgggcaaggacatgggcgcccagtacgccgctgccagcccggcctgggcggccgcgcaacaaaggagtcacccggcgatgagccccggcacccccggaccgaccatgggcagatcccagggcagcccaatggatccaatggtgatgaagagacctcagttgtatggcatgggcagtaaccctcattctcagcctcagcagagcagtccgtacccaggaggttcctatggccctccaggcccacagcggtatccaattggcatccagggtcggactcccggggccatggccggaatgcagtaccctcagcagcaggactctggagatgccacatggaaagaaacattctggttgatgccacctcagtatggacagcaaggtgtgagtggttactgccagcagggccaacagccatattacagccagcagccgcagcccccgcacctcccaccccaggcgcagtatctgccgtcccagtcccagcagaggtaccagccgcagcaggacatgtctcaggaaggctatggaactagatctcaacctcctctggcccccggaaaacctaaccatgaagacttgaacttaatacagcaagaaagaccatcaagtttaccagatctgtctggctccattgatgacctccccacgggaacggaagcaactttgagctcagcagtcagtgcatccgggtccacgagcagccaaggggatcagagcaacccggcgcagtcgcctttctccccacatgcgtcccctcatctctccagcatcccggggggcccatctccctctcctgttggctctcctgtaggaagcaaccagtctcgatctggcccaatctctcctgcaagtatcccaggtagtcagatgcctccgcagccacccgggagccagtcagaatccagttcccatcccgccttgagccagtcaccaatgccacaggaaagaggttttatggcaggcacacaaagaaaccctcagatggctcagtatggacctcaacagacaggaccatccatgtcgcctcatccttctcctgggggccagatgcatgctggaatcagtagctttcagcagagtaactcaagtgggacttacggtccacagatgagccagtatggaccacaaggtaactactccagacccccagcgtatagtggggtgcccagtgcaagctacagcggcccagggcccggtatgggtatcagtgccaacaaccagatgcatggacaagggccaagccagccatgtggtgctgtgcccctgggacgaatgccatcagctgggatgcagaacagaccatttcctggaaatatgagcagcatgacccccagttctcctggcatgtctcagcagggagggccaggaatggggccgccaatgccaactgtgaaccgtaaggcacaggaggcagccgcagcagtgatgcaggctgctgcgaactcagcacaaagcaggcaaggcagtttccccggcatgaaccagagtggacttatggcttccagctctccctacagccagcccatgaacaacagctctagcctgatgaacacgcaggcgccgccctacagcatggcgcccgccatggtgaacagctcggcagcatctgtgggtcttgcagatatgatgtctcctggtgaatccaaactgcccctgcctctcaaagcagacggcaaagaagaaggcactccacagcccgagagcaagtcaaagaagtccagctcctccaccactactggggagaagatcacgaaggtgtacgagctggggaatgagccagagagaaagctctgggtcgaccgatacctcaccttcatggaagagagaggctctcctgtctcaagtctgcctgccgtgggcaagaagcccctggacctgttccgactctacgtctgcgtcaaagagatcgggggtttggcccaggttaataaaaacaagaagtggcgtgagctggcaaccaacctaaacgttggcacctcaagcagtgcagcgagctccctgaaaaagcagtatattcagtacctgtttgcctttgagtgcaagatcgaacgtggggaggagcccccgccggaagtcttcagcaccggggacaccaaaaagcagcccaagctccagccgccatctcctgctaactcgggatccttgcaaggcccacagaccccccagtcaactggcagcaattccatggcagaggttccaggtgacctgaagccacctaccccagcctccacccctcacggccagatgactccaatgcaaggtggaagaagcagtacaatcagtgtgcacgacccattctcagatgtgagtgattcatccttcccgaaacggaactccatgactccaaacgccccctaccagcagggcatgagcatgcccgatgtgatgggcaggatgccctatgagcccaacaaggacccctttgggggaatgagaaaagtgcctggaagcagcgagccctttatgacgcaaggacagatgcccaacagcagcatgcaggacatgtacaaccaaagtccctccggagcaatgtctaacctgggcatggggcagcgccagcagtttccctatggagccagttacgaccgaaggcatgaaccttatgggcagcagtatccaggccaaggccctccctcgggacagccgccgtatggagggcaccagcccggcctgtacccacagcagccgaattacaaacgccatatggacggcatgtacgggcccccagccaagcgccacgagggcgacatgtacaacatgcagtacagcagccagcagcaggagatgtacaaccagtatggaggctcctactcgggcccggaccgcaggcccatccagggccagtacccgtatccctacagcagggagaggatgcagggcccggggcagatccagacacacggaatcccgcctcagatgatgggcggcccgctgcagtcgtcctccagtgaggggcctcagcagaatatgtgggcagcacgcaatgatatgccttatccctaccagaacaggcagggccctggcggccctacacaggcgcccccttacccaggcatgaaccgcacagacgatatgatggtacccgatcagaggataaatcatgagagccagtggccttctcacgtcagccagcgtcagccttatatgtcgtcctcagcctccatgcagcccatcacacgcccaccacagccgtcctaccagacgccaccgtcactgccaaatcacatctccagggcgcccagcccagcgtccttccagcgctccctggagaaccgcatgtctccaagcaagtctccttttctgccgtctatgaagatgcagaaggtcatgcccacggtccccacatcccaggtcaccgggccaccaccccaaccacccccaatcagaagggagatcacctttcctcctggctcagtagaagcatcacaaccagtcttgaaacaaaggcgaaagattacctccaaagatatcgttactcctgaggcgtggcgtgtgatgatgtcccttaaatcaggtcttttggctgagagtacgtgggctttggacactattaatattcttctgtatgatgacagcactgttgctactttcaatctctcccagttgtctggatttctcgaacttttagtcgagtactttagaaaatgcctgattgacatttttggaattcttatggaatatgaagtgggagaccccagccaaaaagcacttgatcacaacgcagcaaggaaggatgacagccagtccttggcagacgattctgggaaagaggaggaagatgctgaatgtattgatgacgacgaggaagacgaggaggatgaggaggaagacagcgagaagacagaaagcgatgaaaagagcagcatcgctctgactgccccggacgccgctgcagacccaaaggagaagcccaagcaagccagtaagttcgacaagctgccaataaagatagtcaaaaagaacaacctgtttgttgttgaccgatctgacaagttggggcgtgtgcaggagttcaatagtggccttctgcactggcagctcggcgggggtgacaccaccgagcacattcagactcactttgagagcaagatggaaattcctcctcgcaggcgcccacctccccccttaagctccgcaggtagaaagaaagagcaagaaggcaaaggcgactctgaagagcagcaagagaaaagcatcatagcaaccatcgatgacgtcctctctgctcggccaggggcattgcctgaagacgcaaaccctgggccccagaccgaaagcagtaagtttccctttggtatccagcaagccaaaagtcaccggaacatcaagctgctggaggacgagcccaggagccgagacgagactcctctgtgtaccatcgcgcactggcaggactcgctggctaagcgatgcatctgtgtgtccaatattgtccgtagcttgtcattcgtgcctggcaatgatgccgaaatgtccaaacatccaggcctggtgctgatcctggggaagctgattcttcttcaccacgagcatccagagagaaagcgagcaccgcagacctatgagaaagaggaggatgaggacaagggggtggcctgcagcaaagatgagtggtggtgggactgcctcgaggtcttgagggataacacgttggtcacgttggccaacatttccgggcagctagacttgtctgcttacacggaaagcatctgcttgccaattttggatggcttgctgcactggatggtgtgcccgtctgcagaggcacaagatccctttccaactgtgggacccaactcggtcctgtcgcctcagagacttgtgctggagaccctctgtaaactcagtatccaggacaataatgtggacctgatcttggccactcctccatttagtcgtcaggagaaattctatgctacattagttaggtacgttggggatcgcaaaaacccagtctgtcgagaaatgtccatggcgcttttatcgaaccttgcccaaggggacgcactagcagcaagggccatagctgtgcagaaaggaagcattggaaacttgataagcttcctagaggatggggtcacgatggcccagtaccagcagagccagcacaacctcatgcacatgcagcccccgcccctggaaccacctagcgtagacatgatgtgcagggcggccaaggctttgctagccatggccagagtggacgaaaaccgctcggaattccttttgcacgagggccggttgctggatatctcgatatcagctgtcctgaactctctggttgcatctgtcatctgtgatgtactgtttcagattgggcagttatgacataagtgagaaggcaagcatgtgtgagtgaagattagagggtcacatataactggctgttttctgttcttgtttatccagcgtaggaagaaggaaaagaaaatctttgctcctctgccccattcactatttaccaattgggaattaaagaaataattaatttgaacagttatgaaattaatatttgctgtctgtgtgtataagtacatcctttggggttttttttttctcttttttttaaccaaagttgctgtctagtgcattcaaaggtcactttttgttcttcacagatctttttaatgttctttcccatgttgtattgcatttttgggggaagcaaattgactttaaagaaaaaagttgtggcaaaagatgctaagatgcgaaaatttcaccacactgagtcaaaaaggtgaaaaattatccatttcctatgcgttttactcctcagagaatgaaaaaaactgcatcccatcacccaaagttctgtgcaatagaaatttctacagatacaggtataggggctcaaggaggtatgtcggtcagtagtcaaaactatgaaatgatactggtttctccacaggaatatggttccattaggctgggagcaaaaacaatgttttttaagattgagaatacatacctgacaacgatccggaaactgctcctcaccactcccgtcatgcctgctgtcggcgtttgaccttccacgtgacagttcttcacaattcctttcatcattttttaaatattttttttactgcctatgggctgtgatgtatatagaagttgtacattaaacataccctcatttttttcttttcttttttttttttttttttagtacaaagttttagtttctttttcatgatgtggtaactacgaagtgatggtagatttaaataattttttatttttattttatatattttttcattagggccatatctccaaaaaaagaaagaaaaaatacaaaaaacaaaaacaaaaaaaaaagagggtaatgtacaagtttctgtatgtataaagtcatgctcgatttcaggagagcagctgatcacaatttgcttcatgaatcaaggtgtggaaatggttatatatggattgatttagaaaatggttaccagtacagtcaaaaaagagaaaatgaaaaaaatacaactaaaaggaagaaacacaacttcaaagatttttcagtgatgagaatccacatttgtatttcaagataatgtagtttaaaaaaaaaaaaaagaaaaaaacttgatgtaaattcctccttttcctctggcttaatgaatatcatttattcagtataaaatctttatatgttccacatgttaagaataaatgtacattaaatcttgttaagcactgtgatgggtgttcttgaatactgttctagtttccttaaagtggtttcctagtaatcaagttatttacaagaaataggggaatgcagcagtgtattcacattataaaaccctacatttggaagagacctttaggggttacctactttagagtggggagcaacagtttgattttctcaaattacttagctaattagtctttctttgaagcaattaactctaacgacattgaggtatgatcattttcagtatttatgggaggtggctgctgacccacttgaggtgagatctcagaagcttaactggcctgaaaatgtaacattctgccttttactaactccatcttagtttaatcaaagttcaatctattccttgtttcttctgtgtgcctcagagttattttgcatttagtttactccaccgtgtataatatttatactgtgcaatgttaaaaaagaatctgttatattgtatgtggtgtacatagtgcaaagtgatgatttctatttcagggcatattatggttctcatattccttcctacctggtgcacagtagctttttaatactagtcacttctaatttaaactttctcttcctgggtcattgactgttactgtgtaataatcgatttctttgaaactgctgcataattatgctgttagtggacctctacctcttctcttccctctcccaatcacagtatactcagaatccccagcccctcgcatacattgtgtcggttcacattactcacagtaatatatggaagagttagacaagaacatgcagttacagtcattgtgagacgtgactctccagtgtcacgaggaaaaaaatcatcttttctgcaaacagtctctcatctgtcaactcccacattactgagtcaaacagtcttcttacataacaatgcaaccaaatatatgttgaattaaagacccatttataattctgctttaaatacatctgcttgctaagaacagatttcagtgctccaagcttcaaatatggagatttgtaagagggaattcaatattattctaatttctctcttacagagtacaaataaaaggtgtatacaaactccgaacatatccagtattccaattcctttgtcaatcagaagagtaaaataattaacaaaagactgttgttatggtttgcattgtaaccgatacgcagagtctgaccgttgggcaacaagtttttctatcctgatgcgcaacacagtctctagagactaatccaggaagactttagcctcctttccatattctcacccccgaatcaagatttacagaagcccacgaagaatttacagcctgcttgagatcatcttgcctataaactgagttattgctttgtcctaaaaattagtcggtttttttttttctatgaggcttttcagaaatttacaggatgcccagactttacatgtgtaccaaaaaaaaaaaaaagataaaaaataaaggtgcaaagaaagtttagtattttggaatggtgctataaagttgaaaaaaaaaaaa

SEQ ID NO: 9 - protein, 118 aa, Homo sapiens
MAAPPEPGEPEERKSLKLLGFLDVENTPCARHSILYGSLGSVVAGFGHFLFTSRIRRSCDVGVGGFILVTLGCWFHCRYNYAKQRIQERIAREEIKKKILYEGTHLDPERKHNGSSSN

SEQ ID NO: 10 - DNA, 1019 bp, Homo sapiens
ggtggagtcgcggagtagtcctcatggccgccccgccggagcccggtgagcccgaggagaggaagtcccttaagctcctaggatttttagatgttgaaaatactccctgcgcccggcattcaatattgtatggttcattaggatctgttgtggctggctttggacattttttgttcactagtagaattagaagatcatgtgatgttggagtaggagggtttatcttggtgactttgggatgctggtttcattgtaggtataattatgcaaagcaaagaatccaggaaagaattgccagagaagaaattaaaaagaagatattatatgaaggtacccacctcgatcctgaaagaaaacacaacggcagcagcagcaattgaacaatcttgagcatagaagtcaatgtaaacgaagttaagatcaaccacataaaacatttcatgtgcaataagctctcaatcaagtaaataaagtttaagttgtagtcatttttttcccacacttgtgtggaatgaaaacttgccagtttattctggccctgtgtctactgccaggatagcattcttacgtgttacatatagtggacttgtcatccttaaaatgtgaacagaatttattggcagtgtggcaaagaattataaaacatagtgtttaatgtacttggagtttccttgtagtagtaagtatagagtttgatgataagtaaacgtcccttaacaaaaacctcaaccttattactatcccattaaaaaacagcaaatacttactgagttcttgtaagagctaatgtcattgtaagatttaaaactaagggcttttatcactttgcaaattattttttaaatgcattcatcatttgacagtgttctctcatttcttaaaatgcgagtcatcttccaaaagagttgtttttaactgccctaaacatttttggggaagtatgcagggtttaaatttttaagtataattagttctgaattaaaatatgcaaaaaaaaaaaaaaaaaaaaaaaaaaaaaa

SEQ ID NO: 11 - protein, 404 aa, Homo sapiens
MAMVTGGWGGPGGDTNGVDKAGGYPRAAEDDSASPPGAASDAEPGDEERPGLQVDCVVCGDKSSGKHYGVFTCEGCKSFFKRSIRRNLSYTCRSNRDCQIDQHHRNQCQYCRLKKCFRVGMRKEAVQRGRIPHSLPGAVAASSGSPPGSALAAVASGGDLFPGQPVSELIAQLLRAEPYPAAAGRFGAGGGAAGAVLGIDNVCELAARLLFSTVEWARHAPFFPELPVADQVALLRLSWSELFVLNAAQAALPLHTAPLLAAAGLHAAPMAAERAVAFMDQVRAFQEQVDKLGRLQVDSAEYGCLKAIALFTPDACGLSDPAHVESLQEKAQVALTEYVRAQYPSQPQRFGRLLLRLPALRAVPASLISQLFFMRLVGKTPIETLIRDMLLSGSTFNWPYGSGQ

SEQ ID NO: 12 - DNA, 1804 bp, Homo sapiens
gtgcagcccgtgccccccgcgcgccggggccgaatgcgcgccgcgtagggtcccccgggccgagaggggtgcccggagggaagagcgcggtgggggcgccccggccccgctgccctggggctatggccatggtgaccggcggctggggcggccccggcggcgacacgaacggcgtggacaaggcgggcggctacccgcgcgcggccgaggacgactcggcctcgccccccggtgccgccagcgacgccgagccgggcgacgaggagcggccggggctgcaggtggactgcgtggtgtgcggggacaagtcgagcggcaagcattacggtgtcttcacctgcgagggctgcaagagctttttcaagcgaagcatccgccgcaacctcagctacacctgccggtccaaccgtgactgccagatcgaccagcaccaccggaaccagtgccagtactgccgtctcaagaagtgcttccgggtgggcatgaggaaggaggcggtgcagcgcggccgcatcccgcactcgctgcctggtgccgtggccgcctcctcgggcagccccccgggctcggcgctggcggcagtggcgagcggcggagacctcttcccggggcagccggtgtccgaactgatcgcgcagctgctgcgcgctgagccctaccctgcggcggccggacgcttcggcgcagggggcggcgcggcgggcgcggtgctgggcatcgacaacgtgtgcgagctggcggcgcggctgctcttcagcaccgtggagtgggcgcgccacgcgcccttcttccccgagctgccggtggccgaccaggtggcgctgctgcgcctgagctggagcgagctcttcgtgctgaacgcggcgcaggcggcgctgcccctgcacacggcgccgctactggccgccgccggcctccacgccgcgcctatggccgccgagcgcgccgtggctttcatggaccaggtgcgcgccttccaggagcaggtggacaagctgggccgcctgcaggtcgactcggccgagtatggctgcctcaaggccatcgcgctcttcacgcccgacgcctgtggcctctcagacccggcccacgttgagagcctgcaggagaaggcgcaggtggccctcaccgagtatgtgcgggcgcagtacccgtcccagccccagcgcttcgggcgcctgctgctgcggctccccgccctgcgcgcggtccctgcctccctcatctcccagctgttcttcatgcgcctggtggggaagacgcccattgagacactgatcagagacatgctgctgtcggggagtaccttcaactggccctacggctcgggccagtgaccatgacggggccacgtgtgctgtggccaggcctgcagacagacctcaagggacagggaatgctgaggcctcgaggggcctcccggggcccaggactctggcttctctcctcagacttctattttttaaagactgtgaaatgtttgtcttttctgttttttaaatgatcatgaaaccaaaaagagactgatcatccaggcctcagcctcatcctccccaggacccctgtccaggatggagggtccaatcctaggacagccttgttcctcagcacccctagcatgaacttgtgggatggtggggttggcttccctggcatgatggacaaaggcctggcgtcggccagaggggctgctccagtgggcaggggtagctagcgtgtgccaggcagatcctctggacacgtaacctatgtcagacactacatgatgactcaaggccaataataaagacatttcctacctgca

SEQ ID NO: 13 - protein, 589 aa, Homo sapiens
MCGPFLKDILHLAEHQGTQSEEKPYTCGACGRDFWLNANLHQHQKEHSGGKPFRWYKDRDALMKSSKVHLSENPFTCREGGKVILGSCDLLQLQAVDSGQKPYSNLGQLPEVCTTQKLFECSNCGKAFLKSSTLPNHLRTHSEEIPFTCPTGGNFLEEKSILGNKKFHTGEIPHVCKECGKAFSHSSKLRKHQKFHTEVKYYECIACGKTFNHKLTFVHHQRIHSGERPYECDECGKAFSNRSHLIRHEKVHTGERPFECLKCGRAFSQSSNFLRHQKVHTQVRPYECSQCGKSFSRSSALIQHWRVHTGERPYECSECGRAFNNNSNLAQHQKVHTGERPFECSECGRDFSQSSHLLRHQKVHTGERPFECCDCGKAFSNSSTLIQHQKVHTGQRPYECSECRKSFSRSSSLIQHWRIHTGEKPYECSECGKAFAHSSTLIEHWRVHTKERPYECNECGKFFSQNSILIKHQKVHTGEKPYKCSECGKFFSRKSSLICHWRVHTGERPYECSECGRAFSSNSHLVRHQRVHTQERPYECIQCGKAFSERSTLVRHQKVHTRERTYECSQCGKLFSHLCNLAQHKKIHT

SEQ ID NO: 14 - DNA, 2368 bp, Homo sapiens
ctaaagctagtggatgtgaagtggtatctcattatggttttggttttcatactcctcatgtttaaggatgctgaacttcttttcatatgcttattggccatttgtgtatatatcttcttttagagaaatgtctatttaagtcctttgacccatttctgtgtccttacccctggtgaggtctcccttattctgttgcttggctggtccctatcctgccaatagtaatgggcccttcttcaccctgatgatggccctgttggcctgtcagcaatccctgggacctcttcttgggtgtgaattcctgggtaacatttctaatgaagtcaaccattcccaccaagtggaattcttagttaactggcatttctctactttcaggttcttggcaatggagtagagggtgagggggcccatcccaagcagaatgtttctgtagaagtgttacaggtcaggatccctaatgcagatccttccaccaagaaagctaactcctgtgacatgtgtgggccattcttgaaagacattttgcacctggctgagcatcagggaacacagtctgaggagaaaccctacacatgtggagcatgtgggagagacttttggttgaatgcaaaccttcaccagcaccagaaggagcacagtggagggaagccctttagatggtacaaggacagggacgcacttatgaagagctctaaagtccacctgtcagagaaccccttcacttgcagggaaggtgggaaggtcatcctgggcagctgtgacctcctccagcttcaagctgttgacagtgggcagaagccatattccaatcttgggcagcttccagaagtctgtaccacacagaaactcttcgagtgcagcaactgtggaaaagccttcctgaagagctccactctccccaaccatctgagaactcactctgaagagataccatttacatgcccaacaggtggaaatttcttagaggagaaatcaatccttggtaataaaaagtttcacactggggaaataccccatgtgtgtaaggagtgtgggaaggcctttagtcactcatctaagctgaggaagcaccagaaatttcacactgaagtaaaatattatgagtgcattgcatgtgggaaaaccttcaaccacaaactcacatttgttcatcatcagagaattcactcaggtgaaagaccttatgagtgtgatgaatgtgggaaagccttcagtaacagatcacacctcattcggcatgagaaagttcacactggagaaaggccttttgagtgcctgaaatgtggaagagccttcagccaaagctccaatttccttcggcatcagaaagttcacacacaggtaagaccttatgagtgcagtcaatgtggtaaatccttcagccgaagctctgctctcattcagcactggagagttcacactggagaaagaccgtatgaatgcagtgaatgtggaagagcttttaacaataactccaaccttgctcagcaccagaaagttcacaccggagaacggccttttgagtgcagtgaatgtggaagagacttcagccaaagctcccatctccttcgacatcagaaagttcacactggagaacggccttttgaatgctgtgattgtggtaaagccttcagtaatagctccaccctcatccagcaccagaaagtacatactgggcaaaggccttatgagtgcagcgaatgtaggaaatccttcagccgcagctccagcctgattcagcactggagaattcacactggagaaaagccttacgagtgtagtgagtgtgggaaagcctttgctcacagctccactctcattgaacactggagagttcacacaaaagaaaggccttatgagtgcaatgaatgtgggaaattctttagccaaaactccattctcattaagcatcagaaagttcatactggagaaaagccttataaatgcagtgaatgtgggaaattctttagccgaaaatccagccttatttgtcactggagagttcacactggagaaaggccttacgaatgcagtgaatgtgggagagcctttagcagtaactcccacctggttcgtcatcagagagttcacacacaagaaaggccctatgagtgcatccagtgtggaaaagcctttagtgaaagatctacacttgttcggcaccagaaagttcacaccagagaaaggacttatgagtgtagccagtgtgggaaactcttcagccatctttgtaaccttgcacagcataaaaagattcatacctgagtggagccttatggaagtggtctttgtgagaaaatcttcagccaagtcaaacttcatgcagcagaatccccataccagaaaaattacctccatgctttag

SEQ ID NO: 15 - protein, 1270 aa, Homo sapiens
MTDDNSDDKIEDELQTFFTSDKDGNTHAYNPKSPPTQNSSASSVNWNSANPDDMVVDYETDPAVVTGENISLSLQGVEVFGHEKSSSDFISKQVLDMHKDSICQCPALVGTEKPKYLQHSCHSLEAVEGQSVEPSLPFVWKPNDNLNCAGYCDALELNQTFDMTVDKVNCTFISHHAIGKSQSFHTAGSLPPTGRRSGSTSSLSYSTWTSSHSDKTHARETTYDRESFENPQVTPSEAQDMTYTAFSDVVMQSEVFVSDIGNQCACSSGKVTSEYTDGSQQRLVGEKETQALTPVSDGMEVPNDSALQEFFCLSHDESNSEPHSQSSYRHKEMGQNLRETVSYCLIDDECPLMVPAFDKSEAQVLNPEHKVTETEDTQMVSKGKDLGTQNHTSELILSSPPGQKVGSSFGLTWDANDMVISTDKTMCMSTPVLEPTKVTFSVSPIEATEKCKKVEKGNRGLKNIPDSKEAPVNLCKPSLGKSTIKTNTPIGCKVRKTEIISYPRPNFKNVKAKVMSRAVLQPKDAALSKVTPRPQQTSASSPSSVNSRQQTVLSRTPRSDLNADKKAEILINKTHKQQFNKLITSQAVHVTTHSKNASHRVPRTTSAVKSNQEDVDKASSSNSACETGSVSALFQKIKGILPVKMESAECLEMTYVPNIDRISPEKKGEKENGTSMEKQELKQEIMNETFEYGSLFLGSASKTTTTSGRNISKPDSCGLRQIAAPKAKVGPPVSCLRRNSDNRNPSADRAVSPQRIRRVSSSGKPTSLKTAQSSWVNLPRPLPKSKASLKSPALRRTGSTPSIASTHSELSTYSNNSGNAAVIKYEEKPPKPAFQNGSSGSFYLKPLVSRAHVHLMKTPPKGPSRKNLFTALNAVEKSRQKNPRSLCIQPQTAPDALPPEKTLELTQYKTKCENQSGFILQLKQLLACGNTKFEALTVVIQHLLSEREEALKQHKTLSQELVNLRGELVTASTTCEKLEKARNELQTVYEAFVQQHQAEKTERENRLKEFYTREYEKLRDTYIEEAEKYKMQLQEQFDNLNAAHETSKLEIEASHSEKLELLKKAYEASLSEIKKGHEIEKKSLEDLLSEKQESLEKQINDLKSENDALNEKLKSEEQKRRAREKANLKNPQIMYLEQELESLKAVLEIKNEKLHQQDIKLMKMEKLVDNNTALVDKLKRFQQENEELKARMDKHMAISRQLSTEQAVLQESLEKESKVNKRLSMENEELLWKLHNGDLCSPKRSPTSSAIPLQSPRNSGSFPSPSISPR

SEQ ID NO: 16 - DNA, 6435 bp, Homo sapiens
aaagggggcggcagcgccggcggagcggaggcgggtctcacgtgggccagcgcagagcctgcggaagggacggatgcggatctcgtcgctgtcaccttgaaagtgaccgaggggcttgactgtggactccttacgccgcccacccgggcccggcggtcccagccttctcgcagggccccttctcagcagaagcaagcggggccgagaaagcgggtggaatagggttgctgcaggtcccaaagacccctcgtggcgcctcgctactttctgcagcttgtttgcactttttcacgctctagaaaaatctcatcttaattaagggaacaacaaatcatttaatcttcagagcatcttagactgaaaacctttcaactgtgctgaaaaacctagaagacagaccattttgcccaccctctcatttaaaaggaattgaagaagaaataaaatggcagaggtttaaggttactattcaggatgactgatgataattcagatgataaaatagaagatgaattgcaaaccttctttaccagtgataaagatggaaatacacatgcatacaacccgaaatcaccacctacacaaaactcttcagccagcagtgtgaactggaattctgccaacccagatgacatggtggttgattatgaaactgaccctgctgtagttactggtgaaaatatttctttaagccttcagggtgttgaagtatttggtcatgaaaagtcttctagtgatttcattagtaagcaggtgttagatatgcataaagattctatttgtcagtgtcctgcacttgtaggtactgagaagcccaaatatctgcaacacagttgtcattccctagaagcagttgagggccagagtgttgagccatctttgccttttgtgtggaagcctaatgacaatttgaactgtgcaggctactgtgatgccttggagctaaaccaaacatttgacatgacagtggataaagttaactgcacctttatatcacatcatgccatcggaaagagtcagtccttccatactgctggaagcctgccaccaactggtaggagaagtggaagtacatcttctttatcctattccacttggacatcttcccattctgataagacgcatgcaagagaaactacttatgatagagaaagctttgaaaaccctcaagtcacaccatcagaagcccaagacatgacttacacagcattttctgatgtggtgatgcaaagtgaggtttttgtttcagatattggaaatcagtgtgcatgttcttcaggaaaggtcaccagtgagtacacagatggatcacaacaaagactagttggagaaaaggagacacaagcactaacaccagtttctgatggcatggaagtccccaatgattctgcattacaagagttcttttgtttatcccatgatgaatccaatagcgaaccacattcacagagctcatacaggcacaaggaaatgggccaaaatctgagagagacagtgtcctattgtcttattgatgatgaatgccctttaatggtgccagcttttgataagagcgaagctcaagtgctgaacccagagcataaagtcactgagactgaagacacacaaatggtctccaaaggaaaggatttgggaacccaaaatcatacctcagaattgattctaagtagcccgccaggacaaaaggtgggctcgtcatttggactgacttgggatgcaaatgatatggtcattagcacagacaaaacgatgtgcatgtcaacaccagtcctagaacccacaaaagtaaccttttctgtttcaccgattgaagcgacggagaaatgtaagaaagtggagaagggtaatcgagggcttaaaaacataccagactcgaaggaggcacctgtgaacctgtgtaaacccagtttaggaaaatcaacaatcaaaacgaataccccaataggctgcaaagttagaaaaactgaaattataagttacccaagaccaaacttcaagaatgtcaaagcaaaagttatgtctagagcagtgttgcagcccaaagatgctgctttatcaaaggtcacgcccagacctcagcagaccagtgcctcatcaccctcatcagtgaattcaagacaacaaacagtcttgagcagaacaccgagatctgacttgaatgcagacaaaaaagcagaaattctaattaacaagacacataagcagcagtttaataaactcattactagccaggctgtgcatgttacaactcattctaaaaatgcttcacacagggttccaagaacaacatctgccgtgaaatcgaatcaggaagatgttgacaaagccagttcttctaactcagcatgcgagaccgggtccgtttctgcgttgtttcagaagatcaaaggcatactccctgttaaaatggaaagtgcagaatgtttggaaatgacctatgttcccaacattgataggattagccctgaaaagaagggtgaaaaagaaaatgggacatctatggaaaaacaagagctgaaacaagagattatgaatgagacttttgaatatggttctctgtttttgggctctgcttcaaaaacaacgaccacctcaggtaggaatatatccaagcctgactcctgcggtttgaggcaaatagctgctccaaaagccaaagtggggccccctgtttcctgtttgaggcggaacagtgacaatagaaatcccagtgctgatcgagccgtatctcctcagaggatcaggcgtgtgtccagttctggaaagcctacatccttgaaaactgcacagtcgtcatgggtgaatttgcctagaccacttcctaaatccaaagcatctttgaaaagtcctgcgctgcggaggacaggaagcaccccctcaatagccagcacccacagtgagctgagcacttacagcaacaattctggtaatgccgctgtcatcaaatatgaggagaaacctccaaaaccagcatttcagaatggttcctcaggatccttttatttgaagcctttggtatccagggctcatgttcacttgatgaaaactcctccaaaaggtccttcgagaaaaaatttatttacagctcttaatgcagttgaaaagagcaggcaaaagaatcctcgaagcttatgtatccagccacagacagctcccgatgcgctgccccctgagaaaacacttgaattgacgcaatataaaacaaaatgtgaaaaccaaagtggatttatcctgcagctcaagcagcttcttgcctgtggtaataccaagtttgaggcattgacagttgtgattcagcacctgctgtctgagcgggaggaagcactgaaacaacacaaaaccctatctcaagaacttgttaacctccggggagagctagtcactgcttcaaccacctgtgagaaattagaaaaagccaggaatgagttacaaacagtgtatgaagcattcgtccagcagcaccaggctgaaaaaacagaacgagagaatcggcttaaagagttttacaccagggagtatgaaaagcttcgggacacttacattgaagaagcagagaagtacaaaatgcaattgcaagagcagtttgacaacttaaatgctgcgcatgaaacctctaagttggaaattgaagctagccactcagagaaacttgaattgctaaagaaggcctatgaagcctccctttcagaaattaagaaaggccatgaaatagaaaagaaatcgcttgaagatttactttctgagaagcaggaatcgctagagaagcaaatcaatgatctgaagagtgaaaatgatgctttaaatgaaaaattgaaatcagaagaacaaaaaagaagagcaagagaaaaagcaaatttgaaaaatcctcagatcatgtatctagaacaggagttagaaagcctgaaagctgtgttagagatcaagaatgagaaactgcatcaacaggacatcaagttaatgaaaatggagaaactggtggacaacaacacagcattggttgacaaattgaagcgtttccagcaggagaatgaagaattgaaagctcggatggacaagcacatggcaatctcaaggcagctttccacggagcaggctgttctgcaagagtcgctggagaaggagtcgaaagtcaacaagcgactctctatggaaaacgaggagcttctgtggaaactgcacaatggggacctgtgtagccccaagagatcccccacatcctccgccatccctttgcagtcaccaaggaattcgggctccttccctagccccagcatttcacccagatgacacctccccaaagtccacagactctctgaaagcattttgatgcaggtctgcaggactgaccccaaggaggaacgtgggcacaagaggtatatcagcacacgtgtgatcaccgtagggtaactggagcgtcaccaccggcggaatcgcagcttctgagactggaactctggaggaagacttttgcctccgtccaaaagattcctccaaaaaaagatttaaaaaaagatttcggcatcgacacggacgttgttgcacaaagcacttaaagaacgagagcatcttgttcattgcctttttcacctaagcatagggggaaaaactctcagggccctattaagatttataacctttgtaatgttcttcaccacagacaccttcttgtgagttttcagtctgactgtgggggtggggggtgtgaatgaaatggatgtcacagagtgtcatgtgtctgatgcagcctcctctgctgtgtattaaatgtcaaaatctgaatatatctggatatgtactaatcaaataataatcaatcaatcagcatatacatttcagccaaagccatagaagaaaaagcaatagttgcttgaattatgatcatctaccaccaactctgctcagccctgtaacagggtagggagagggtataacaggaagagctttgacttgtccctgtctatacattctctgtatcttttgggggtaacttcttggcagtttttcagtgttcagccatgtcagttgaaactagatttttctgtagattttttacttacccatgtgagcctaacactatcctgtaattcattttctcaggctatgtgtaaatgtagaaccctaatttttctataaaaaaacaaactaactaactaactgtgtaaagaaagaaaaagggaagtaccaatgggtttttccaccttatttttacctttgatctacccttgcagatttaacctgtcttcttccctcccattattctcattttccttttacctttctccaccatccagagccacaaaagcaaaccttctacctcctacctacttttctctgggacaaggataaaggaatatgattttccagagccccagagccagctcatcttccaggtgctgaaaccactttccaaataaactaaagcctggatttgatattacaaattttgggaaatcttagaataaagaacgagaacaaggaagtcattggctagtataattaagaaaggtaggattcagtgcttaccgatgatgcagtacttgatagaagaaaacagtctgggaggatagcgctcatttttcagttaccctttaaggagtccctttgtctttgggaaagtagcagaatggtccgcttctttcccatgagtggaaaatgtggcttgtccaactctcctccaggttgcatttcagtttctttccaaaacttattacctcccctaatcctgagactttggaaaaggtggaaggaagaactgttgctttatctccccctccctgcatgtgtcaacattgtgatgtcagtatttactaatctacattcagtggctgtacaaataacagctgtagtaagaagagattcaggatgctagaggtgaatatttgggtcatttacatgtacactacatagcaagttgatactcatgttgcatgttcttttaaattagtgattttgtgtcttaagtctttaacttccaatacttcatcatgtatgtaaccttccatgtttgcttctgataaatggaaatgtaggttcactgccacttcatgagatatctctgctcacgcttccaagttgttctcaatgacattagccaaagttgggtttgccattcatcccctaggcatggtaaatcttgtgttgttccctgctgtcctccgtattacgtgaccggcaaataaatctcatagcagttaatataaaacatctttggaggatgggagagaacaggagggaagatgggaaacaaaatagagaattcttaagattttgtttaaaccaaatgtttcatgtagaatgcaaaatgttggcacgtcaaaaatatgaatgtgtagacaactgtagttgtgctcagtttgtagtgatgggaagtgtattttactctgatcaaataaataatgctggaatactcaagaattgcaaaaaaaaaaaaaaaaa

SEQ ID NO: 17 - protein, 1156 aa, Homo sapiens
MFPAAPSPRTPGTGSRRGPLAGLGPGSTPRTASRKGLPLGSAVSSPVLFSPVGRRSSLSSRGTPTRMFPHHSITESVNYDVKTFGSSLPVKVMEALTLAEVDDQLTINIDEGGWACLVCKEKLIIWKIALSPITKLSVCKELQLPPSDFHWSADLVALSYSSPSGEAHSTQAVAVMVATREGSIRYWPSLAGEDTYTEAFVDSGGDKTYSFLTAVQGGSFILSSSGSQLIRLIPESSGKIHQHILPQGQGMLSGIGRKVSSLFGILSPSSDLTLSSVLWDRERSSFYSLTSSNISKWELDDSSEKHAYSWDINRALKENITDAIWGSESNYEAIKEGVNIRYLDLKQNCDGLVILAAAWHSADNPCLIYYSLITIEDNGCQMSDAVTVEVTQYNPPFQSEDLILCQLTVPNFSNQTAYLYNESAVYVCSTGTGKFSLPQEKIVFNAQGDSVLGAGACGGVPIIFSRNSGLVSITSRENVSILAEDLEGSLASSVAGPNSESMIFETTTKNETIAQEDKIKLLKAAFLQYCRKDLGHAQMVVDELFSSHSDLDSDSELDRAVTQISVDLMDDYPASDPRWAESVPEEAPGFSNTSLIILHQLEDKMKAHSFLMDFIHQVGLFGRLGSFPVRGTPMATRLLLCEHAEKLSAAIVLKNHHSRLSDLVNTAILIALNKREYEIPSNLTPADVFFREVSQVDTICECLLEHEEQVLRDAPMDSIEWAEVVINVNNILKDMLQAASHYRQNRNSLYRREESLEKEPEYVPWTATSGPGGIRTVIIRQHEIVLKVAYPQADSNLRNIVTEQLVALIDCFLDGYVSQLKSVDKSSNRERYDNLEMEYLQKRSDLLSPLLSLGQYLWAASLAEKYCDFDILVQMCEQTDNQSRLQRYMTQFADQNFSDFLFRWYLEKGKRGKLLSQPISQHGQLANFLQAHEHLSWLHEINSQELEKAHATLLGLANMETRYFAKKKTLLGLSKLAALASDFSEDMLQEKIEEMAEQERFLLHQETLPEQLLAEKQLNLSAMPVLTAPQLIGLYICEENRRANEYDFKKALDLLEYIDEEEDININDLKLEILCKALQRDNWSSSDGKDDPIEVSKDSIFVKILQKLLKDGIQLSEYLPEVKDLLQADQLGSLKSNPYFEFVLKANYEYYVQGQI

SEQ ID NO: 18 - DNA, 4170 bp, Homo sapiens
ctcttcccttaggtgtttaagttccgcgcgcaggccaggctgcaacctgacggccagatccctcgctgtcctagtcgctgctccttggagtcatgttcccagccgccccttctccgcggaccccgggtaccgggtcccgaaggggcccgctggccggactcgggcccggctccacgccccggacggctagcaggaagggtctgcccctggggtctgcagtcagctccccagtgctcttctcgccggtcggccggcgtagctcgctaagctcgcggggaacaccaacacgaatgttcccacaccactccataactgagtctgtgaactatgatgtgaaaacgtttggatcttctcttcctgttaaagtcatggaagccctaacattggctgaagtcgatgaccagctgaccattaacatagatgaaggtggatgggcttgtctggtgtgcaaagagaagctcattatttggaagattgctctgtcacctattactaagttatccgtttgcaaagaacttcagctgccacctagtgatttccactggagtgccgacttagtggctctttcttactcttctccctcaggtgaagcacattctactcaggctgttgctgtcatggttgccaccagagaaggatctatccgctattggccaagccttgctggtgaagatacctacacagaggcttttgtagattcgggaggtgataagacttacagtttcctaacagcagtgcagggaggaagttttattttgtcttcatcaggaagccaactaattcggttgatacctgagagctcaggaaagattcatcagcatatcctgcctcaggggcaaggcatgctttcaggaattggtcgaaaagtttcttctctttttggaattttatctcctagtagtgatctcacactttcaagtgttctctgggatagagagagatcaagcttttatagcctgacgagttcaaacatcagtaaatgggaattagatgattcttcagaaaagcatgcatacagttgggatataaatagagccctgaaggaaaacattaccgatgctatttggggatctgaaagtaactatgaagctattaaagaaggagtcaacattcgatatttggacttgaagcaaaactgtgatgggctggtgattttggcagcagcatggcactcagcagacaatccatgtctcatctattactctctgataacaatagaagataatggttgccaaatgtcagatgcagttactgtagaagtcactcaatataatccaccttttcagtctgaagacctgattttgtgtcagttgacggtcccaaacttttcaaaccagactgcctatctgtataacgaaagtgctgtctatgtgtgctccacaggaactgggaaattttctcttccccaggagaaaattgtctttaatgcacaaggagatagtgttttaggtgctggtgcctgtggtggtgttcctatcattttttctagaaacagtggactggtgtctattacttcaagggaaaatgtgtctatattggcagaagacttggaagggtctttagcatcttcagttgctggaccaaacagtgagagtatgatttttgagaccactacaaagaatgaaactatagcccaggaagataaaatcaagttgctgaaagctgcctttctgcaatactgcagaaaagatttaggtcatgctcaaatggtggttgatgagctcttttcctctcactctgatttggattctgattctgaactagacagggcagttacccaaatcagtgtagacctgatggatgactacccagcatctgacccacggtgggctgagtctgtccctgaggaagcacctgggttcagcaatacgtcactgattatccttcaccagctagaagacaagatgaaagctcactcttttcttatggactttattcatcaagttggcttatttggacgtctaggcagttttccagttagagggacaccgatggccactcgactgttgctctgtgagcatgccgaaaagctgtcagccgccattgttctcaagaaccaccactcccggctttctgaccttgtcaacacagccatattgattgctttgaacaagagggagtatgaaatcccatccaacctgactcctgcagatgtctttttcagggaggtatcccaagtagataccatctgtgagtgcttactggagcatgaggagcaagtcttgagggatgcacctatggattccattgaatgggctgaagtggtgatcaatgtgaacaatattctcaaggatatgctgcaggctgctagtcattatcgccaaaatagaaactctttgtatagaagagaagaatcactagaaaaagaacctgaatatgttccatggacggcaacaagtggtcctggtggcatccgaacggtaataatacgccagcatgagattgtcctgaaggtggcttatccacaggcagacagcaacctccgaaacatcgtgaccgagcagctggtagccctgatcgattgcttcctggatggttatgtttctcagcttaagtctgtggataaatccagtaatcgggaaagatatgacaatctggagatggaatacctacagaaaagatcagatctcttatctcctcttctttcactaggccagtacctgtgggctgcttctctagcagagaaatactgtgactttgatatattggtacaaatgtgtgagcagactgacaaccagagccgactccagcgctacatgacccagtttgctgatcagaatttttcagactttctcttccgttggtatctggagaaaggaaagcgaggcaaattattatctcagcccatttctcagcatggacagttggcaaattttttgcaagctcatgaacatctcagctggttacatgaaattaatagccaagaattagaaaaggctcatgcaacacttctgggtttggcaaatatggaaactcgttactttgcaaagaagaaaacccttcttggcttgagtaaattggctgcattagcttcagacttttcagaggatatgctacaagaaaaaattgaagaaatggctgagcaggagcgctttctactgcatcaggagaccctacctgaacagctgctggcggagaaacagctaaatctcagtgcgatgccagtattgactgcaccacaactcattggtctatatatctgtgaagaaaatagaagagctaatgaatatgatttcaagaaagctttggacttgttggaatatattgatgaggaagaagatataaatataaatgatctaaaactggaaatcctttgcaaagctcttcagagagataactggtccagttctgatggcaaagatgatccaattgaagtatctaaagacagtatatttgtgaagatcttacagaaacttttaaaagatggcattcagctcagtgagtacttaccggaggtgaaagacctgctacaagcggatcagcttggaagcttaaagtccaatccttacttcgagtttgttttgaaagcaaattatgaatattatgttcagggacaaatataactttttctaaaaatggccattgtttatgaaatctgtataagtgtgtccttatacaaattttaggccataaacaagtgtaagtttgtacaatttcataacatgtatagctgagtttttatactttatatgtaggaagctaatataaaatagttatgtaactgtgattttggttttcagttatgtgacttgttttttccacctgaaatgtgtcagttgttgttcctgtactcggtgccctttctttttactctcacgtggtcccaggttctggagttcttgtcctggttctagctgctcacatgtacaaatcacttctaggcctcagtttctgcgactatgaaaattactagattgcactagcttgtctctaaaattgctgtgactccagatactttgcactgaagagaatctagggtgtttgatatctgtttcagttagggctaatgggaaatgtctagtaagataaatgtcaacttttgctgacttattatgagatgaaaaaccaaaggagagtgggcctaactcatgtgagcttgataactgatgaactcattgggagcattttaaacttttctacataaataataaatgagcactaatgaaagta

SEQ ID NO: 19 - protein, 620 aa, Homo sapiens
MGPLQFRDVAIEFSLEEWHCLDTAQRNLYRNVMLENYSNLVFLGIVVSKPDLIAHLEQGKKPLTMKRHEMVANPSVICSHFAQDLWPEQNIKDSFQKVILRRYEKRGHGNLQLIKRCESVDECKVHTGGYNGLNQCSTTTQSKVFQCDKYGKVFHKFSNSNRHNIRHTEKKPFKCIECGKAFNQFSTLITHKKIHTGEKPYICEECGKAFKYSSALNTHKRIHTGEKPYKCDKCDKAFIASSTLSKHEIIHTGKKPYKCEECGKAFNQSSTLTKHKKIHTGEKPYKCEECGKAFNQSSTLTKHKKIHTGEKPYVCEECGKAFKYSRILTTHKRIHTGEKPYKCNKCGKAFIASSTLSRHEFIHMGKKHYKCEECGKAFIWSSVLTRHKRVHTGEKPYKCEECGKAFKYSSTLSSHKRSHTGEKPYKCEECGKAFVASSTLSKHEIIHTGKKPYKCEECGKAFNQSSSLTKHKKIHTGEKPYKCEECGKAFNQSSSLTKHKKIHTGEKPYKCEECGKAFNQSSTLIKHKKIHTREKPYKCEECGKAFHLSTHLTTHKILHTGEKPYRCRECGKAFNHSATLSSHKKIHSGEKPYECDKCGKAFISPSSLSRHEIIHTGEKP

SEQ ID NO: 20 - DNA, 1990 bp, Homo sapiens
agacaccaggacccctggaagcctagaaatgggaccattgcaatttagagatgtggccatagaattctctctggaggagtggcattgcctggacactgcacagcggaatctatataggaatgtgatgttagagaactacagtaacctggtcttccttggtattgttgtctctaagccagacctgatcgcccatctggagcaaggaaaaaaacctttgactatgaagagacatgagatggtagccaacccctcagttatatgttctcattttgcccaagatctttggccagagcagaacataaaagattctttccaaaaagtgatactgagaagatatgaaaaacgtggacatggaaatttacagttaataaaaaggtgtgaaagtgtagatgagtgtaaggtgcacacaggaggttataatggacttaaccagtgtagtacaactacccagagcaaagtatttcaatgtgataaatatgggaaagtctttcataaattttcaaattcaaatagacataatataagacatactgaaaaaaaacctttcaaatgcatagaatgtggcaaagcttttaaccagttctcaacccttataacacataagaaaattcatactggagagaaaccctacatttgtgaagaatgtggcaaagcctttaagtactcctctgcccttaatacacataagagaattcatactggagagaaaccatacaagtgtgataaatgtgacaaagcctttattgcatcctcaacccttagtaaacatgagatcattcatactggaaagaaaccctacaagtgtgaagaatgtggcaaagcttttaaccaatcctcgacacttactaaacataagaaaattcatactggagagaaaccctacaaatgtgaagaatgtggcaaagcttttaaccaatcctcaacacttactaaacataagaaaattcatactggagagaagccctacgtttgtgaagaatgtggcaaagcctttaagtactcccgtatccttactacacataagagaattcatactggagagaaaccatacaagtgtaataaatgtggcaaagcctttattgcatcctcaacccttagtagacatgagttcattcatatgggaaagaaacattacaaatgtgaagaatgtggcaaagccttcatttggtcctcagtcctaactagacataagagagttcatactggagagaagccctacaaatgtgaagaatgtggcaaagcctttaagtactcctctacccttagttcacataagagaagtcatactggagagaaaccctacaaatgtgaagaatgtggcaaagcctttgttgcatcctcaacccttagtaaacatgagatcattcatactggaaagaaaccctacaagtgtgaagaatgtggcaaagcttttaaccagtcctcatcccttactaaacataagaaaattcatactggagagaaaccctacaaatgtgaagaatgtggcaaagcttttaaccagtcctcttcccttactaaacataagaaaattcatactggagagaaaccctacaaatgtgaagaatgtggcaaagcttttaaccagtcctcaacccttattaaacataagaaaattcatactagagagaaaccctacaaatgtgaagaatgtggcaaagcttttcacctatccacacaccttactacacataagatacttcatactggagagaaaccttatagatgtagagaatgtggcaaagcttttaaccattctgcaaccctttcttcacataagaaaatccattctggagagaaaccatacgagtgtgataaatgtggcaaagcctttatttcaccctcaagccttagtagacatgagataattcatactggggagaaaccctagaagtgtgaagaatgtggcaaagccttcaagtggtcctcacaccttactatacactgagagttctgaacttactctgtaaccatcccaaactcctcccag

SEQ ID NO: 21 - protein, 303 aa, Homo sapiens
MAAVHDLEMESMNLNMGREMKEELEEEEKMREDGGGKDRAKSKKVHRIVSKWMLPEKSRGTYLERANCFPPPVFIISISLAELAVFIYYAVWKPQKQWITLDTGILESPFIYSPEKREEAWRFISYMLVHAGVQHILGNLCMQLVLGIPLEMVHKGLRVGLVYLAGVIAGSLASSIFDPLRYLVGASGGVYALMGGYFMNVLVNFQEMIPAFGIFRLLIIILIIVLDMGFALYRRFFVPEDGSPVSFAAHIAGGFAGMSIGYTVFSCFDKALLKDPRFWIAIAAYLACVLFAVFFNIFLSPAN

SEQ ID NO: 22 - DNA, 912 bp, Homo sapiens
atggctgctgttcatgatctggagatggagagcatgaatctgaatatggggagagagatgaaagaagagctggaggaagaggagaaaatgagagaggatgggggaggtaaagatcgggccaagagtaaaaaggtccacaggattgtctcaaaatggatgctgcccgaaaagtcccgaggaacatacttggagagagctaactgcttcccgcctcccgtgttcatcatctccatcagcctggccgagctggcagtgtttatttactatgctgtgtggaagcctcagaaacagtggatcacgttggacacaggcatcttggagagtccctttatctacagtcctgagaagagggaggaagcctggaggtttatctcatacatgctggtacatgctggagttcagcacatcttggggaatctttgtatgcagcttgttttgggtattcccttggaaatggtccacaaaggcctccgtgtggggctggtgtacctggcaggagtgattgcagggtcccttgccagctccatctttgacccactcagatatcttgtgggagcttcaggaggagtctatgctctgatgggaggctattttatgaatgttctggtgaattttcaagaaatgattcctgcctttggaattttcagactgctgatcatcatcctgataattgtgttggacatgggatttgctctctatagaaggttctttgttcctgaagatgggtctccggtgtcttttgcagctcacattgcaggtggatttgctggaatgtccattggctacacggtgtttagctgctttgataaagcactgctgaaagatccaaggttttggatagcaattgctgcatatttagcttgtgtcttatttgctgtgtttttcaacattttcctatctccagcaaactga

SEQ ID NO: 23 - protein, 150 aa, Homo sapiens
MAARGVIAPVGESLRYAEYLQPSAKRPDADVDQQRLVRSLIAVGLGVAALAFAGRYAFRIWKPLEQVITETAKKISTPSFSSYYKGGFEQKMSRREAGLILGVSPSAGKAKIRTAHRRVMILNHPDKGGSPYVAAKINEAKDLLETTTKH

SEQ ID NO: 24 - DNA, 2404 bp, Homo sapiens
agtctccgggccgccttgccatggctgcccgtggtgtcatcgctccagttggcgagagtttgcgctacgctgagtacttgcagccctcggccaaacggccagacgccgacgtcgaccagcagagactggtaagaagtttgatagctgtaggcctgggtgttgcagctcttgcatttgcaggtcgctacgcatttcggatctggaaacctctagaacaagttatcacagaaactgcaaagaagatttcaactcctagcttttcatcctactataaaggaggatttgaacagaaaatgagtaggcgagaagctggtcttattttaggtgtaagcccatctgctggcaaggctaagattagaacagctcataggagagtcatgattttgaatcacccagataaaggtggatctccttacgtagcagccaaaataaatgaagcaaaagacttgctagaaacaaccaccaaacattgatgcttaaggaccacactgaaggaaaaaaaaagaggggacttcaaaaaaaaaaaaaaagccctgcaaaatattctaaaacatggtcttcttaattttctatatggattgaccacagtcttatcttccaccattaagctgtataacaataaaatgttaatagtcttgctttttattatcttttaaagatctccttaaattctataactgatcttttttcttattttgtttgtgacattcatacatttttaagatttttgttatgttctgaattcccccctacacacacacacacacacacacacacacacgtgcaaaaaatatgatcaagaatgcaattgggatttgtgagcaatgagtagacctcttattgtttatatttgtaccctcattgtcaatttttttttagggaatttgggactctgcctatataaggtgttttaaatgtcttgagaacaagcactggctgatacctcttggagatatgatctgaaatgtaatggaatttattaaatggtgtttagtaaagtaggggttaaggacttgttaaagaaccccactatctctgagaccctatagccaaagcatgaggacttggagagctactaaaatgattcaggtttacaaaatgagccctgtgaggaaaggttgagagaagtctgaggagtttgtatttaattatagtcttccagtactgtatattcattcattactcattctacaaatatttattgaccccttttgatgtgcaaggcactatcgtgcgtcccctgagagttgcaagtatgaagcagtcatggatcatgaaccaaaggaacttatatgtagaggaaggataaatcacaaatagtgaatactgttagatacagatgatatattttaaaagttcaaaggaagaaaagaatgtgttaaacactgcatgagaggaggaataagtggcatagagctaggctttagaaaagaaaaatattccgataccatatgattggtgaggtaagtgttattctgagatgagaattagcagaaatagatatatcaatcggagtgattagagtgcagggtttctggaaagcaaggtttggacagagtggtcatcaaaggccagccctgtgacttacactgcattaaattaatttcttagaacatagtccctgatcattatcactttactattccaaaggtgagagaacagattcagatagagtgccagcattgtttcccagtattcctttacaaatcttgggttcattccaggtaaactgaactactgcattgtttctatcttaaaatactttttagatatcctagatgcatctttcaacttctaacattctgtagtttaggagttctcaaccttggcattattgacatgttaggccaaataattttttttgtgggaggtctcttgtgcgttttagatgattagcaataatccctgacctgttatctactaaagactagtcgtttctcatcagttgtgacaacaaaaatggttccagatattgccaaatgccctttagaggacagtaatcgcccccagttgagaaccatttcagtaaaactttaattactattttttcttttggtttataaaataatgatcctgaattaaattgatggaaccttgaagtcgataaaatatatttcttgctttaaagtccccatacgtgtcctactaattttctcatgctttagtgttttcacttttctcctgttatccttgtacctaagaatgccatcccaatccccagatgtccacctgcccaaagtctaggcatagctgaaggccaagctaaaatgtatccctctttttctggtacatgcagcaaaagtaatatgaattatcagctttctgagagcaggcattgtatctgtcttgtttggtgttacattggcacccaataaatatttgttgagcgaaaaaaaaaaaaaaa

(19) United States
(12) Patent Application Publication
(10) Pub. No.: US 2014/0296104 A1
(43) Pub. Date: Oct. 2, 2014

(54) Genes Differentially Expressed by Cumulus Cells and Assays Using Same to Identify Pregnancy Competent Oocytes

(71) Applicant: GEMA DIAGNOSTICS, INC., Ann Arbor, MI (US)

(72) Inventors: Jose B. Cibelli, East Lansing, MI (US); Amy E. Iager, Ada, MI (US); Hasan H. Otu, Istanbul (TR)

(21) Appl. No.: 14/351,750

(22) PCT Filed: Oct. 15, 2012
(86) PCT No.: PCT/US2012/060307

§ 371 Date: Apr. 14, 2014

Related U.S. Application Data
(60) Provisional application No. 61/547,403, filed on Oct. 14, 2011.
(60) Provisional application No. 61/581,219, filed on Dec. 29, 2011.

(51) Int. Cl. C12Q 1/68

(52) U.S. Cl. CPC C12Q 1/6888
USPC 506/9; 435/6.12; 435/6.11; 435/7.1; 506/16; 506/17; 506/18

(57) ABSTRACT

A genetic means of identifying “pregnancy competent” oocytes is provided. The means comprises detecting the level of expression of one or more genes that are expressed at characteristic levels (upregulated or downregulated) in cumulus cells derived from pregnancy competent oocytes. This characteristic gene expression level, or pattern referred to herein as the “pregnancy signature”, also can be used to identify subjects with underlying conditions that impair or prevent the development of a viable pregnancy, e.g., pre-menopausal condition, other hormonal dysfunction, ovarian dysfunction, ovarian cyst, cancer or other cell proliferation disorder, autoimmune disease and the like. In preferred embodiments the pregnancy signature will comprise one or more of FG-F12, (Hs00374427_m1), GPR137B (Hs00162803_m1), SLC2A9 (Hs00417125_m1), ARID IB (Hs00368175_m1), NR2F6 (Hs00172870_m1), ZNF132 (Hs01036387_m1), FAM36A (Hs00831105_s1), ZNF93 (Hs01656246,,s1), RHBDL2 (Hs00384848_m1), DNAJC15 (Hs00387763_m1), MTUS1 (Hs00826834_m1), ND NUP133 (Hs00217272_m1), or their orthologs, splice or allelic variants.

### CROSS REFERENCE TO RELATED APPLICATIONS

This PCT application claims priority to U.S. Provisional Application Ser. No. 61/547,403 filed on Oct. 14, 2011 and U.S. Provisional Application Ser. No. 61/581,219 filed on Dec. 29, 2011.

This application also relates to PCT application WO/2011/060080, published May 19, 2011, U.S. provisional application Ser. No. 61/388,296 filed Sep. 30, 2010; U.S. provisional application Ser. No. 61/387,313 and 61/387,286 both filed Sep. 28, 2010; U.S. provisional application Ser. No. 61/360,556 filed on Jul. 1, 2010 and U.S. provisional application Ser. No. 61/259,783 filed on Nov. 10, 2009. The contents of all of the identified provisional and non-provisional applications is incorporated herein by reference in its entirety.

### FIELD OF THE INVENTION

The present invention identifies a pregnancy signature gene set containing 12 genes, i.e., FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1), wherein the expression of one or more of these genes by cumulus cells correlates to the competency of an oocyte associated therewith, or from the same female donor.

Based on this discovery, the present invention provides methods and test kits for identifying human oocytes which are potentially suitable for use in IVF procedures by detecting the level of expression of one or more of these 12 genes or corresponding polypeptides consisting of FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1).

Based on this discovery, the present invention provides arrays or test kits containing one or more of these genes or polypeptides or primers or antibodies that provide for the detection and/or quantification of the level of expression of one or more of these 12 genes or corresponding polypeptides consisting of FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1). For example, such test kits may contain antibodies that specifically detect one or more of the gene products encoded by these 12 genes and one or more detectable label. Also, such test kits may comprise primers that provide for the specific amplication of one or more of these 12 genes in a sample such as a nucleic acid sample obtained from cumulus cells which are associated with oocytes potentially to be used for fertilization or IVF procedures.

Based on the foregoing, the present invention further provides genetic methods of identifying female subjects and materials (microarrays, test kits) for use therein, preferably human females, having impaired fertility function, e.g., as a result of impaired ovarian function because of age (menopause), underlying disease condition or drug therapy by analyzing the expression of one or more of these 12 specific genes on cumulus cells obtained from oocytes isolated from said female subject.

Also, the invention provides methods of evaluating the efficacy of a putative fertility or hormonal treatment by assessing its effect on the expression of one, two, three, four, five, six, seven, eight, nine, ten, eleven or all 12, or any combination thereof, of 12 specific genes, i.e., FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1), by cumulus cells of a female subject receiving this fertility or hormonal treatment.

### BACKGROUND OF THE INVENTION

Currently, there is no reliable commercially available genetic or non-genetic procedure for identifying whether a female subject produces oocytes that are “pregnancy competent”, i.e., oocytes which when fertilized by natural or artificial means are capable of giving rise to embryos that in turn are capable of yielding viable offspring when transferred to an appropriate uterine environment. Rather, conventional fertility assessment methods assess fertility e.g., based on hormonal levels, visual inspection of numbers and quality of oocytes, surgical or non-invasive (MRI) inspection of the female reproduction system organs, and the like. Often, when a woman has a problem in producing a viable pregnancy after a prolonged duration, e.g., more than a year, the diagnosis may be an “unexplained” fertility problem and the woman advised to simply keep trying or to seek other options, e.g., adoption or surrogacy.

Perhaps in part of the lack of a means for identifying pregnancy competent oocytes, the success rate for assisted reproductive technology (ART), pregnancy and birth rates following in vitro fertilization (IVF) attempts remain low. Subjective morphological parameters are still a primary criterion to select healthy embryos used for in IVF and ICSI programs. However, such criteria do not truly predict the competence of an embryo. Many studies have shown that a combination of several different morphologic criteria leads to more accurate embryo selection. Morphological criteria for embryo selection are assessed on the day of transfer, and are principally based on early embryonic cleavage (25-27 h post insemination), the number and size of blastomeres on day two, day three, or day five, fragmentation percentage and the presence of multi-nucleation in the 4 or 8 cell stage (Fenwick et al., Hum Reprod, 17, 407-12. (2002).

A recent study has shown that the selection of oocytes for insemination does not improve outcome of ART as compared to the transfer of all available embryos, irrespective of their quality (La Sala et al., Fertil Steril. (2008)).

There is a need to identify viable embryos with the highest implantation potential to increase IVF success rates, reduce the number of embryos for fresh replacement and lower multiple pregnancy rates. For all these reasons, several biomarkers for embryo selection are currently being investigated (Haouzi et al., Gynecol Obstet Fertil, 36, 730-742. (2008); He et al., Nature, 444, 12-3. (2006)).

As embryos that result in pregnancy differ in their metabolic profiles compared to embryos that do not, some studies are trying to identify a molecular signature that can be detected by non-invasive evaluation of the embryo culture medium (Brison et al., Hum Reprod, 19, 2319-24. (2004); Gardner et al., Fertil Steril, 76, 1175-80. (2001); Sakkas and Gardner, Curr Opin Obstet Gynecol, 17, 283-8 (2005); Seli et al., Fertil Steril, 88, 1350-7. (2007); Zhu et al. Fertil Steril. (2007).

Genomics are also providing vital knowledge of genetic and cellular function during embryonic development. McKenzie et al., Hum Reprod, 19, 2869-74. (2004); Feuerstein et al., Hum Reprod, 22, 3069-77 have reported, that the expression of several genes in cumulus cells, such as cyclooxygenase 2 (COX2), was indicative of oocyte and embryo quality. In addition Gremlin 1 (GREM1), hyaluronic acid synthase 2 (HAS2), steroidogenic acute regulatory protein (STAR), stearoyl-coenzyme A desaturase 1 and 5 (SCD1 and 5), amphiregulin (AREG) and pentraxin 3 (PTX3) have also been reported to be positively correlated with embryo quality (Zhang et al., Fertil Steril, 83 Suppl 1, 1169-79. (2005)). More recently, the expression of glutathione peroxidase 3 (GPX3), chemokine receptor 4 (CXCR4), cyclin D2 (CCND2) and catenin delta 1 (CTNND1) in human cumulus cells have been shown to be inversely correlated with embryo quality, based on early-cleavage rates during embryonic development (van Montfoort et al., (2008) MoI Hum Reprod, 14, 157-68. (2008)).

Also Cillo et al., Reprod. 134:645-50 (2007) suggests a correlation between the expression of certain cumulus genes, i.e., HAS2, GREM1 and PTX3 and oocyte quality and embryo development. Still further Assidi et al. Biol. Reprod. 79(2) 209-222 (2008) suggest a correlation as to the expression of certain cumulus genes, i.e., EGFR, CD44, HAS2, PTSG2 and BTC and oocyte quality and development of embryos therefrom. Further, Bettegowda et al., Biol. Reprod. 79(2):301-309 (2008) suggest a correlation as to the expression of certain proteinase cathepsin genes and bovine oocyte quality and development of offspring therefrom.

In addition, a patent was recently issued to Zhang et al. (Aug. 11, 2009) claims the detection of pentraxin 3 and a BCL-2 member on cumulus cells to assess oocyte quality. Also, US20040058975 published on Mar. 25, 2004 teaches that antagonism of the EP2 receptor and/or cycloxygenase COX-2 promotes cumulus cell proliferation and oocyte development.

Also, while early cleavage has been shown to be a reliable biomarker for predicting pregnancy (Lundin et al., Hum Reprod, 16, 2652-7. (2001); Van Montfoort et al., Hum Reprod, 19, 2103-8 (2004; Yang et al., Fertil Steril, 88, 1573-8 (2007)), little has been reported correlating gene expression profiles of cumulus cells with respect to pregnancy outcome (but see Assou et al., Mol Hum Reprod. 2008 December; 14(12):711-9. Epub 2008 Nov. 21).

Therefore, notwithstanding the foregoing, providing alternative and more predictive methods for identifying oocytes suitable for use in IVF procedures and in identifying the genetic bases of fertility problems in women would be highly desirable. In particular an identification of other genes, and biomarkers, the expression of which by cumulus cells correlates to pregnancy competency of oocytes and test kits and assays using same would be highly desirable as this could enhance the outcome of IVF procedures.

These methods and test kits would in addition provide for the identification of women with oocyte related fertility problems, which is desirable as such fertility problems may correlate to other health issues that preclude pregnancy, e.g., cancer, menopausal condition, hormonal dysfunction, ovarian cyst, or other underlying disease or health related problems.

### BRIEF DESCRIPTION AND OBJECTS OF THE INVENTION

The present invention relates to a method for selecting a competent oocyte, e.g., one that gives rise to a fertilized embryo that yields a viable pregnancy comprising a step of measuring the expression level of any combination of one of 12 genes selected from the group consisting of FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1) by a cumulus cell associated with an oocyte or from an oocyte from the same female donor and comparing said gene expression to a suitable control, e.g., cumulus cells of female donors with normal oocytes, i.e., which give rise to viable pregnancies.

The present invention also relates to a method for selecting a competent embryo, comprising a step of measuring the expression level of specific genes in a cumulus cell surrounding the embryo, wherein said genes include or consist of genes selected from the group consisting of FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1).

The present invention also relates to a method for selecting a competent oocyte or a competent embryo, comprising a step of measuring in a cumulus cell surrounding said oocyte or said embryo the expression level of one or more genes selected from the FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1).

Aberrant expression levels of one or more of these genes is predictive of a non competent oocyte or embryo due to early embryo arrest.

As discussed infra, it has been found that the level of expression of these genes by a cumulus cell of a woman donor correlates to the likelihood that an oocyte associated with said cumulus cell or derived from the same subject are “pregnancy competent” when fertilized by natural or artificial means. These genes and expression levels constitute what Applicants refer to as the “pregnancy signature”. In addition the pregnancy signature may further include one or more of the genes disclosed in Applicant's prior applications identified supra.

It is a related object of the invention to provide a novel method of determining whether an individual has a genetic associated fertility problem which potentially renders the individual's oocytes unsuitable for use in IVF methods based on the detected level of expression of one or more genes or corresponding polypeptides which constitute the “pregnancy signature.” The genes and gene products which constitute the pregnancy signature are again preferably selected from those contained in FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1).

It is another object of the invention to provide a method of evaluating the efficacy of a female fertility treatment which comprises: treating a female subject putatively having a problem that prevents or inhibits her from having a “viable pregnancy” and isolating at least one oocyte from said female subject and cells associated therewith after said fertility treatment; isolating at least one cumulus cell associated with said isolated oocyte, and detecting the level of expression of at least one gene selected from FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1), or their orthologs, splice or allelic variants that is expressed at a characteristic level of expression in “pregnancy competent” oocytes; and determining the putative efficacy of said fertility treatment based on whether said gene is expressed at a level characteristic of “pregnancy competent” oocytes as a result of treatment.

It is another specific object of the invention to provide novel methods of treating infertility by modulating the expression of one or more genes that constitute the pregnancy signature. These methods include the administration of compounds that agonize or antagonize the expression of one or more of the genes selected from FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1), or their orthologs, splice or allelic variants and their splice or allelic variants.

It is another object of the invention to provide animal models for evaluating the efficacy of putative fertility treatments comprising identifying genes which are expressed at characteristic levels in cumulus cells associated with pregnancy competent oocytes of a non-human animal, e.g., a non-human primate; and assessing the efficacy of a putative fertility treatment in said non-human animal based on its effect on said gene expression levels, i.e., whether said treatment results in said gene expression levels better mimicking gene expression levels observed in cumulus cells associated with pregnancy competent oocytes, (“pregnancy signature”). i.e. one or more of the 12 genes selected from FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1), or their orthologs, splice or allelic variants.

### DETAILED DESCRIPTION OF THE FIGURES

FIG. 1 contains a flow chart of methods used to identify the subject “pregnancy signature” i.e., 12 genes the expression of which on cumulus cells correlates to the pregnancy competency or ability of an oocyte associated with said cumulus cell or from the same female human or other mammalian donor to be capable of fertilization and when used in an IVF procedure capable of giving rise to a viable fetus and live offspring

FIG. 2 shows the predictive value and specificity of the subject gene detection methods according to Youdun's index.

### DETAILED DESCRIPTION OF THE INVENTION

Prior to discussing the invention in more detail, the following definitions are provided. Otherwise all words and phrases in this application are to be construed by their ordinary meaning, as they would be interpreted by an ordinary skilled artisan within the context of the invention.

“Pregnancy-competent oocyte”: refers to a female gamete or egg that when fertilized by natural or artificial means is capable of yielding a viable pregnancy when it is comprised in a suitable uterine environment.

“The term “competent embryo” similarly refers to an embryo with a high implantation rate leading to pregnancy. The term “high implantation rate” means the potential of the embryo when transferred in uterus, to be implanted in the uterine environment and to give rise to a viable fetus, which in turn develops into a viable offspring absent a procedure or event that terminates said pregnancy.

“Viable-pregnancy”: refers to the development of a fertilized oocyte when contained in a suitable uterine environment and its development into a viable fetus, which in turn develops into a viable offspring absent a procedure or event that terminates said pregnancy.

“Cumulus cell” refers to a cell comprised in a mass of cells that surrounds an oocyte. This is an example of an “oocyte associated cell”. These cells are believed to be involved in providing an oocyte some of its nutritional and or other requirements that are necessary to yield an oocyte which upon fertilization is “pregnancy competent”.

“Differential gene expression” refer to genes the expression of which varies within a tissue of interest; herein preferably a cell associated with an oocyte, e.g., a cumulus cell.

“Real Time RT-PCR”: refers to a method or device used therein that allows for the simultaneous amplification and quantification of specific RNA transcripts in a sample.

“Microarray analysis”: refers to the quantification of the expression levels of specific genes in a particular sample, e.g., tissue or cell sample.

“Pregnancy signature”: herein preferably refers to the normal level of expression of one or more genes or polypeptides that are selected or encoded by the specific genes selected from the group consisting of FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1). and their orthologs, splice or allelic variants wherein these genes or polypeptides are expressed in normal cumulus cells at levels which correlate to the likelihood that an oocyte that is associated with a cumulus cell which expresses said one or more genes or polypeptides at these characteristic levels are more likely to give rise to a viable pregnancy. Alternatively the signature may include one or more of the genes differentially expressed by cumulus cells the expression of which also correlates to pregnancy competent oocytes which are identified in the patent applications incorporated by reference herein.

“Characteristic level of expression of a cumulus gene” herein with respect to a particular detected expressed nucleic acid sequence or polypeptide means that the particular gene or polypeptide is expressed at levels which are substantially similar to the levels observed in cumulus cells that are associated with a normal cumulus cell or one associated with a normal or developmentally competent oocyte.

By “substantially similar” is meant that the levels of expression of individual genes are preferably within the range of +/−1-5 fold of the level of expression by a normal cumulus cell, more preferably within the range of +/−1-3-fold, still more preferably within the range of +/−1-1.5 fold and most preferably within the range of +/−1.0-1.4, 1.0-1.3, 1.0-1.2 or 1.0-1.1 fold of the detected levels of expression of the gene or polypeptide by a normal cumulus cell.

According to the invention, the oocyte may result from a natural cycle, a modified natural cycle or a stimulated cycle for cIVF or ICSI. The term “natural cycle” refers to the natural cycle by which the female or woman produces an oocyte. The term “modified natural cycle” refers to the process by which, the female or woman produces an oocyte or two under a mild ovarian stimulation with GnRH antagonists associated with recombinant FSH or hMG. The term “stimulated cycle” refers to the process by which a female or a woman produces one ore more oocytes under stimulation with GnRH agonists or antagonists associated with recombinant FSH or hMG.

“Oocyte or cumulus cell determined to possess suitable pregnancy signature or to be pregnancy competent” refers to an oocyte or a cumulus cell associated with the oocyte or an oocyte derived from the same subject at around the same time (within 0-6 months) as the tested cumulus cell which has been determined to express at least one of the genes or polypeptides encoded by the following genes: FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1). or an ortholog or splice or allelic variant thereof in a manner characteristic of the level of expression by a normal cumulus cell. Preferably at least 2 or 3 genes are expressed in a characteristic manner, more preferably at least 3-5 genes, or their allelic or splice variants. It should be understood that if the expression of numerous genes are evaluated in the subject genetic based assays, such as in the order of 10 or more, that a suitable pregnancy signature means that all or substantially all, i.e. at least 70-80% of the detected genes are expressed in a manner characteristic of a normal cumulus cell. For example if the expression of 10 genes is detected at least 7, 8 or 9 of the genes will preferably be expressed at the levels consistent with a normal cumulus cell, i.e. one associated with an oocyte capable of giving rise to a normal embryo and viable pregnancy.

In general with respect to the pregnancy signature the characteristic levels of expression is observed for any combination of the afore-identified 12-gene pregnancy signature set, i.e., any combination of at least 1, 2, 3, 4, 5, 6, 7, 8, 9, 10, 11 or 12 of the afore-identified genes, that are expressed at characteristic levels in cumulus cells, that surround “pregnancy competent” oocytes. This is intended to encompass the level at which the gene is expressed and the distribution of gene expression within cumulus cells analyzed.

“Pregnancy signature gene”: refers to a gene which is expressed at characteristic levels by a cumulus cell, which is associated with a normal or “pregnancy competent” oocyte. These genes are FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1). and their orthologs, splice and allelic variants. These 12 human genes are referenced by their name as well as Accession number. It should be understood that the invention further encompasses detection of allelic and splice variants of these genes and species orthologs.

“Probe suitable for detection of the expression of a pregnancy signature gene or polypeptide” refers to a nucleic acid sequence or sequences or ligand such as an antibody that specifically detects the expression of the transcribed gene or corresponding polypeptide. In a preferred embodiment expression is selected by use of realtime PCR detection methods.

“IVF”: refers to in vitro fertilization.

The term “classical in vitro fertilization” or “cIVF” refers to a process by which oocytes are fertilized by sperm outside of the body, in vitro. IVF is a major treatment in infertility when in vivo conception has failed. The term “intracytoplasmic sperm injection” or “ICSI” refers to an in vitro fertilization procedure in which a single sperm is injected directly into an oocyte. This procedure is most commonly used to overcome male infertility factors, although it may also be used where oocytes cannot easily be penetrated by sperm, and occasionally as a method of in vitro fertilization, especially that associated with sperm donation.

“Zona pellucida” refers to the outermost region of an oocyte.

“Method for detecting differential expressed genes” encompasses any known method for quantitatively evaluating differential gene expression using a probe that specifically detects for the expressed gene transcript or encoded polypeptide. Examples of such methods include indexing differential display reverse transcription polymerase chain reaction (DDRT-PCR; Mahadeva et al, 1998, J. Mol. Biol. 284:1391-1318; WO 94/01582; subtractive mRNA hybridization (See Advanced Mol. Biol.; R. M. Twyman (1999) Bios Scientific Publishers, Oxford, p. 334, the use of nucleic acid arrays or microarrays (see Nature Genetics, 1999, vol. 21, Suppl. 1061) and the serial analysis of gene expression. (SAGE) See e.g., Valculesev et al, Science (1995) 270:484-487) and real time PCR (RT-PCR). For example, differential levels of a transcribed gene in an oocyte cell can be detected by use of Northern blotting, and/or RT-PCR. A preferred method is the CRL amplification protocol refers to the novel total RNA amplification protocol that combines template-switching PCR and T7 based amplification methods. This protocol is well suited for samples wherein only a few cells or limited total RNA is available.

Preferably, the “pregnancy signature” genes are detected by hybridization of RNA or DNA to DNA chips, e.g., filter arrays comprising cDNA sequences or glass chips containing cDNA or in situ synthesized oligonucleotide sequences. Filtered arrays are typically better for high and medium abundance genes. DNA chips can detect low abundance genes. In the exemplary embodiment the sample may be probed with Affymetrix GeneChips comprising genes from the human genome or a subset thereof.

Alternatively, polypeptide arrays comprising the polypeptides encoded by pregnancy signature genes or antibodies that bind thereto may be produced and used for detection and diagnosis.

“EASE” is a gene ontology protocol that from a list of genes forms subgroups based on functional categories assigned to each gene based on the probability of seeing the number of subgroup genes within a category given the frequency of genes from that category appearing on the microarray.

Based on the foregoing the present invention provides a novel method of detecting whether a female, preferably human or non-human mammal, produces “pregnancy competent” oocytes or whether a particular oocyte is pregnancy competent. The method involves detecting the levels of expression of one or more genes in selected from the group consisting of FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1) that are expressed at characteristic levels by cumulus cells associated with (surrounding) oocytes that are “pregnancy competent”, i.e., these oocytes when fertilized by natural or artificial means (IVF), and transferred into a suitable uterine environment are capable of yielding a viable pregnancy, i.e., embryo that develops into a viable fetus and eventually an offspring unless the pregnancy is terminated by some event or procedure, e.g., a surgical or hormonal intervention.

As described herein the inventors have determined a set of 12 genes expressed in cumulus cells that are biomarkers for embryo potential and pregnancy outcome. They demonstrated that genes expression profile of cumulus cells which surrounds oocyte correlated to different pregnancy outcomes, allowing the identification of a specific expression signature of embryos developing toward pregnancy. Their results indicate that analysis of cumulus cells surrounding the oocyte is a non-invasive approach for embryo selection.

The set of 12 predictive genes herein are known human genes. However, the expression of these genes (on cumulus cells) had not heretofore been correlated to oocyte competency or embryo development. Therefore, this invention relates to a method for selecting a competent oocyte, comprising a step of measuring the expression level of specific genes in a cumulus cell surrounding said oocyte, wherein said genes include at least one of the genes selected from the group consisting of FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1).

The methods of the invention may further comprise a step consisting of comparing the expression level of the genes in the sample with a control, wherein detecting differential in the expression level of the genes between the sample and the control is indicative whether the oocyte is competent. The control may consist in sample comprising cumulus cells associated with a competent oocyte or in a sample comprising cumulus cells associated with an unfertilized oocyte.

The methods of the invention are applicable preferably to human women but may be applicable to other mammals (e.g., primates, dogs, cats, pigs, cows) including endangered species wherein IVF procedures are often used in zoos in order to increase population numbers.

The methods of the invention are particularly suitable for assessing the efficacy of an in vitro fertilization treatment. Accordingly the invention also relates to a method for assessing the efficacy of a controlled ovarian hyperstimulation (COS) protocol in a female subject comprising: 1) providing from said female subject at least one oocyte with its cumulus cells; ii) determining by a method of the invention whether said oocyte is a competent oocyte.

Then after such a method, the embryologist may select the competent oocytes and in vitro fertilize them, fur example using a classical in vitro fertilization (cIVF) protocol or under an intracytoplasmic sperm injection (ICSI) protocol.

A further object of the invention relates to a method for monitoring the efficacy of a controlled ovarian hyperstimulation (COS) protocol comprising: 1) isolating from said woman at least one oocyte with its cumulus cells under natural, modified or stimulated cycles; ii) determining by a method of the invention whether said oocyte is a competent oocyte; iii) and monitoring the efficacy of COS treatment based on whether it results in a competent oocyte.

The COS treatment may be based on at least one active ingredient selected from the group consisting of GnRH agonists or antagonists associated with recombinant FSH or hMG.

The present invention also relates to a method for selecting a competent embryo, comprising a step of measuring the expression level of at least one of the 12 genes selected from the group consisting of FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1).

The methods of the invention may further comprise a step consisting of comparing the expression level of the genes in the sample with a control, wherein detecting differential in the expression level of the genes between the sample and the control is indicative whether the embryo is competent. The control may consist in sample comprising cumulus cells associated with an embryo that gives rise to a viable fetus or in a sample comprising cumulus cells associated with an embryo that does not give rise to a viable fetus.

It is noted that the methods of the invention leads to an independence from morphological considerations of the embryo. Two embryos may have the same morphological aspects but by a method of the invention may present a different implantation rate leading to pregnancy.

The methods of the invention are applicable preferably to human women but may be applicable to other mammals, both domesticated ad non-domesticated such as endangered species (e.g. primates, dogs, cats, pigs, cows, tigers, lions, pandas, cheetahs, et al.).

The present invention also relates to a method for determining whether an embryo is a competent embryo, comprising a step consisting of measuring the expression level of specific genes in a cumulus cell surrounding the embryo, wherein said genes include at least one of the 12 genes selected from the group consisting of FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1).

The present invention also relates to a method for determining whether an embryo is a competent embryo, comprising: i) providing an oocyte with its cumulus cells; ii) in vitro fertilizing said oocyte; and iii) determining whether the embryo that results from step ii) is competent by determining by a method of the invention whether said oocyte of step i), is a competent oocyte.

The present invention also relates to a method for selecting a competent oocyte or a competent embryo, comprising a step of measuring in a cumulus cell surrounding said oocyte or said embryo the expression level of one or more genes selected from at least one of the 12 genes selected from the group consisting of FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1). Aberrant expression of one or more of these genes selected my be predictive of a non competent oocyte or embryo, the inability of the embryo being unable to implant or of a non competent oocyte or embryo due to early embryo arrest.

The methods of the invention are particularly suitable for enhancing the pregnancy outcome of a female. Accordingly the invention also relates to a method for enhancing the pregnancy outcome of a female comprising: i) selecting a competent embryo by performing a method of the invention; iii) implanting the embryo selected at step i) in the uterus of said female, wherein said female may or may not be the oocyte donor.

The method as above described will thus help embryologist to avoid the transfer in uterus of embryos with a poor potential for pregnancy outcome. The method as above described is also particularly suitable for avoiding multiple pregnancies by selecting the competent embryo able to lead to an implantation and a viable, full-term pregnancy.

### Methods for Determining the Expression Level of the Genes of the Invention:

Determination of the expression level of the genes in the “pregnancy signature” i.e., at least one of the 12 genes selected from the group consisting of FGF 12, (Hs00374427_m1), GPR137B (Hs00162803_m1), SLC2A9 (Hs00417125_m1), ARID 1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1) can be performed by a variety of techniques. Generally, the expression level as determined is a relative expression level.

More preferably, the determination comprises contacting the sample with selective reagents such as probes, primers or ligands, and thereby detecting the presence, or measuring the amount, of polypeptide or nucleic acids of interest originally in the sample. Contacting may be performed in any suitable device, such as a plate, microtitre dish, test tube, well, glass, column, and so forth. In specific embodiments, the contacting is performed on a substrate coated with the reagent, such as a nucleic acid array or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the sample.

In a preferred embodiment, the expression level may be determined by determining the quantity of mRNA.

Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it is advantageous to use nucleic acids in combination with appropriate means, such as a detectable label, for detecting hybridization. A wide variety of appropriate indicators are known in the art including, fluorescent, radioactive, enzymatic or other ligands (e.g. avidin/biotin).

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate). The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from cumulus cells and subjecting the RNAs to amplification and hybridization to specific probes, more particularly by means of a quantitative or semiquantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a micro sphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labeled and contacted with the microarray in hybridization conditions, leading to the formation of complexes between target nucleic acids that are complementary to probe sequences attached to the microarray surface. The labeled hybridized complexes are then detected and can be quantified or semi-quantified. Labeling may be achieved by various methods, e.g. by using radioactive or fluorescent labeling. Many variants of the microarray hybridization technology are available to the man skilled in the art (see e.g. the review by Hoheisel, Nature Reviews, Genetics, 2006, 7:200-210)

In this context, the invention further provides a DNA chip comprising a solid support which carries nucleic acids that are specific to at least one of the 12 genes selected from the group consisting of FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1).

Other methods for determining the expression level of said genes include the determination of the quantity of proteins encoded by said genes.

Such methods comprise contacting the sample with a binding partner capable of selectively interacting with a marker protein present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e.g., in membrane or microtitre well form); polyvinylchloride (e.g., sheets or microtitre wells); polystyrene latex (e.g., beads or microtitre plates); polyvinylidine fluoride; diazotized paper; nylon membranes; activated beads, magnetically responsive beads, and the like. More particularly, an ELISA method can be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate (s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

Alternatively an immunohistochemistry (IHC) method may be preferred. IHC specifically provides a method of detecting targets in a sample or tissue specimen in situ. The overall cellular integrity of the sample is maintained in IHC, thus allowing detection of both the presence and location of the targets of interest. Typically a sample is fixed with formalin, embedded in paraffin and cut into sections for staining and subsequent inspection by light microscopy. Current methods of IHC use either direct labeling or secondary antibody-based or hapten-based labeling. Examples of known IHC systems include, for example, EnVision™ (DakoCytomation), Powervision® (Immunovision, Springdale, Ariz.), the NBA™ kit (Zymed Laboratories Inc., South San Francisco, Calif.), HistoFine® (Nichirei Corp, Tokyo, Japan).

In particular embodiment, a tissue section (e.g. a sample comprising cumulus cells) may be mounted on a slide or other support after incubation with antibodies directed against the proteins encoded by the genes of interest. Then, microscopic inspections in the sample mounted on a suitable solid support may be performed. For the production of photomicrographs, sections comprising samples may be mounted on a glass slide or other planar support, to highlight by selective staining the presence of the proteins of interest.

Therefore IHC samples may include, for instance: (a) preparations comprising cumulus cells (b) fixed and embedded said cells and (c) detecting the proteins of interest in said cells samples. In some embodiments, an IHC staining procedure may comprise steps such as: cutting and trimming tissue, fixation, dehydration, paraffin infiltration, cutting in thin sections, mounting onto glass slides, baking, deparaffination, rehydration, antigen retrieval, blocking steps, applying primary antibodies, washing, applying secondary antibodies (optionally coupled to a suitable detectable label), washing, counter staining, and microscopic examination.

The invention also relates to a kit for performing the methods as above described, wherein said kit comprises means for measuring the expression level the levels of at least one of the 12 genes selected from the group consisting of FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1) that are indicative whether the oocyte or the embryo is competent.

The invention is further illustrated by the following description of how the inventors determined that the expression of one or more of these 12 genes on a cumulus cell correlates to oocyte competency and embryo development upon implantation and working examples. However, these examples and description should not be interpreted in any way as limiting the scope of the present invention.

The present inventors used accepted statisatical methods to assess specific genes wherein the levels of expression thereof by cumulus cells correlates to the pregnancy competency of an oocyte associated therewith or from the same donor. The methods are summarized below:

Statistical methods and algorithms used to identify the 12 gene signature of the present invention are further described below.

Gene Signature Refinement

We ran TLDAs on 49 (24N; 25F) samples that have been used in microarray profiling with 196 genes that can be represented on the TLDA.

TLDA Output Normalization

Scaling

From the TLDA analysis, we have two sets of output: Ct values (logged expression levels) and dCt values, where for a given sample, each gene's dCt value is calculated by subtracting Ct values of an endogenous control, in this case the 18S endogenous control gene imprinted on all TLDA plates, from the gene's cT value. Since cT values are logarithmic, this corresponds to dividing each gene's expression value by 18S's expression value. In other words, it is the fold change between a gene and 18S. Moving on with these values mean calculating fold change between groups based on genes' fold change with respect to 18S. dCt values are referred to as “scaled”.

Delta Ct Value Normalization

Once scaled, further normalization was done so that 12-gene valued vector for each sample has “length” or “amplitude” 1.

For a given sample, we calculated the “amplitude” or “length” of the 12 valued-vector (this is achieved by summing the square of each gene and then taking the square root) and then divide each gene value by this number.

Prediction Analysis

Following normalization, it was observed that 84 genes showed the same direction of expression in both TLDA and microarray results.

In the prediction analysis, we used the only genes in agreement between Affy and TLDA when genes that are “undetected” in 25 or more samples are filtered out. We found 84 genes to be detected and concordant between Affy and TLDA.

Leave-One-Out-Cross-Validation (L1OXV)

To arrive at the smallest, most predictive set from these 84 genes, Gema executed an iterative strategy called leave-one-out-cross-validation (L1OXV). L1OXV is explained as follows:

In this method, first number of genes in the predictive gene set, say P, is fixed. Then one sample in the training set is left-out and top P genes using the remaining samples that differentiate between N and F are calculated. Using these P genes the sample that is left out is predicted as N or F. This process is cycled through all 33 samples in the training set (leaving one out at a time). The total number of correct predictions is listed as the accuracy of the predictor on the training set.

During L1OXV process, different values for P, number of predictor genes, are tried and for ones that show good L1OXV prediction accuracy, these genes are applied on the validation set. The number of samples correctly predicted in the validation set is reported as prediction accuracy in the validation set. The smallest P that yields high training and validation accuracies are reported as the predictor gene set.

Prediction Analysis Results

Prediction analysis using these 84 confirmed genes and the normalized TLDA values of the 49 samples yielded a 12 gene signature with ˜72% prediction accuracy (35/49 correct predictions—14/24 N's; 21/25 F's correctly predicted). The predictor gene set remained significant using the Fisher's test, permutation test and randomization test (p-value<0.05).

Weighted Average Prediction Algorithm

Signal to Noise Ratio

During the weighted voting approach, we used “signal to noise ratio” (SNR) to assess predictor value of a gene g (Golub et al., 1999). Let μF(g) and μN(g) be the mean value of gene g in F and N sample groups, respectively. Similarly, let σF(g) and σN(g) be the standard deviation of gene g in F and N sample groups, respectively. We define SNR(g)=[μF(g)−μN(g)]/[σF(g)+σN(g)]. This metric defines a neighborhood in RM around ideal gene expression vectors for both groups where M=|F|+|N|, total number of samples in the data set. SNR punishes genes with an expression highly deviant in either group and provides a signed ranking method for a gene's membership. In this case large positive values indicate a good predictor for the F group and large negative values (in absolute value) indicate a good predictor for the N group.

Boundary Value

We also define the boundary between the correlation between idealized expression patterns and a given gene g as B(g)=[μF(g)+μN(g)]/2.

Assume we are given a predictor gene set of P genes G=(g1, g2, . . . , gP), a group of F and N samples and a new sample S to be predicted. The vote of gi, 1≦i≦P, is defined as Vi=SNR(gi) [S(gi)−B(gi)], where S(gi) represents the signal value of gene gi in S. Vi represents how well S(gi) relates to the “behavior” of gi in F and N samples. If Vi is positive, we conclude that based on gi, S is predicted to be F and if Vi is negative gi predicts S as N. Cycling through all genes in the predictor set we obtain P votes and let VF be the sum of all positive votes and VN be the sum of all negative votes. If VF is greater than VN in absolute value, we predict sample S as F; otherwise we predict S as N. Alternatively, one can consider the number of positive versus number of negative votes. If number of positive votes is greater than P/2, then the sample is predicted as F; otherwise it is predicted as N. Finally, both “sum” and “number of votes” criteria can be used in combination for sample prediction.

Prediction Algorithm

The first step in the prediction algorithm is to calculate prediction values for each gene in each sample. These values are calculated by multiplying the SNR of the gene by the difference between the normalized dCt value and the boundary value.

Once prediction values for each gene in each sample is calculated, a total prediction value for each sample is calculated by summing the prediction values of each gene in the sample.

The final prediction is made by using the following logic: If the sum of the Prediction Values for that sample is less than 0 and the count of the positive Prediction Values for each gene in that sample is less than 7, then the sample is an “F”, otherwise “N”.

### Data Analysis

There are various issues to consider such as handling of data points that have a value of 40, calculating fold change, and whether or not to use logged values. Below, we address such issues providing potential solutions.

Scaling: We have two sets of output: Ct values (logged expression levels) and dCt values, where for a given sample, each gene's dC value is calculated by subtracting GAPDH's Ct value from the gene's Ct value. Since Ct values are logarithmic, this corresponds to dividing each gene's expression value by GAPDH's expression value. In other words, it is the fold change between a gene and GAPDH. Moving on with these values mean calculating fold change between groups based on genes' fold change with respect to GAPDH. Since GAPDH is not one of the endogenous controls used on the array, there are no spike-in controls used in TLDA, and small variations in logarithmic scale may imply large differences in real values, we approach this with some caution. Nevertheless, we provide analysis both using scaled and unscaled values. For the remainder of this report unscaled values refer to Ct values as obtained in amplification files and scaled values refer to dCt values obtain by subtracting GAPDH.

Fold Change:

Assuming we have two samples A and B, and gene X's expression values in these samples are aX and bX, respectively. What we see in TLDA output (Ct values) are log(aX) and log(bX). If you want to calculate fold change between these two samples, you would subtract Ct values and take that to power of 2. That is, FC=2 log(aX)−log(bX). The reason for this is the following rules: log p−log q=log(p/q) and 2 log 2p=p. However, since Ct values are reversed, i.e. a smaller value means larger expression, this FC gives you the fold change B/A. To exemplify, if we see a Ct value of 10.8 in A and 12.3 in B, this means this gene is upregulated in A and fold change for B/A is 2 10.8−12.3=2−1.5=0.35. In other words, this gene is upregulated in A by 1/0.35=2.8 times. Another way to arrive this point is first to unlog Ct values and then calculate FC as we know it, except that the direction is reversed, i.e. in Ct world less means more. Hence, we have the expression level for A=2 10.8=1782, the expression level for B=2 12.3=5042, and FC B/A=1782/5042=0.35.

FC values less than 1 are hard to interpret so what we do is we reverse them and put a minus sign. For the above example, instead of saying FC for B/A is 0.35, we say FC for B/A is −1/0.35=−2.8. In all my calculations, we always subtracted F values from N values (if we were using log scale) or divided N values by F values (if we used unlogged values) and calculated FC for F/N. we used negative values to depict FCs less than 1 as explained above.

As if it has not been complicated enough to calculate a simple FC, we have more to think about. The example above contained only two samples, or, you can view it as having one sample in each group. How about if we have more than one sample in each group, as in our case (16 N, 19F)? If you average Ct values, you indeed get a geometric mean of expression levels. If you then subtract averages of Ct values in two groups and then take that to the power of two, this in turn means calculating FC by dividing geometric means of expressions in two groups. The reason for this is the following rules: alogX=logXa and logp+log q=log (pq).

To give an example, assume you have expression levels a, b, and c in group N and d, e, f, and g in group F. What we see in TLDA output is log a, log b, . . . , etc. In order to calculate FC (F/N), if we subtract the average value in F from the average value in N and then take that to power 2, we get the following:

Average in N=⅓[log a+log b+log c]=⅓ log [abc]=log(abc)⅓

Average in F=¼[log d+log e+log f+log g]=¼ log [defg]=log (defg)¼

FC(F/N)=2̂[log(abc)⅓−log(defg)¼]=2̂(log [(abc)⅓/(defg)¼])=(abc)⅓/(defg)¼

Recall that geometric mean of n numbers is nth root of their products. Therefore, we always choose to work with unlogged values. That is, we first took Ct values to the power of 2 and then did our analyses.

40:40 is an arbitrary Ct value considered high enough to represent a gene that has not been detected. However, if you set it to 42 instead of 40, all your results will change. Therefore, we resolved this by first looking at all values that are not 40 and ranked them. For Hasan Genes, this corresponds to ranking 4623 values. We then looked at the bottom 2% of these genes, that is lowest 92 genes; calculated their average and standard deviation, which turned out to be 37.9 and 0.8. We then replaced each 40 by a number randomly chosen between the interval [37.9−0.8, 37.9+0.8].

Outliers: When you manually look at the expression levels, you often see samples that behave as outliers for a given gene. In order to overcome this we removed the highest and lowest expression levels in a group (N or F) when calculating FC. We also repeated this procedure by removing highest two and lowest two samples in each group.

Gene Signature Refinement

We ran TLDAs on 49 (24N; 25F) samples that have been used in microarray profiling with 196 genes that can be represented on the TLDA.

TLDA Output Normalization

Scaling

From the TLDA analysis, we have two sets of output:

Ct values (logged expression levels) and

dCt values, where for a given sample, each gene's dCt value is calculated by subtracting Ct values of an endogenous control, in this case the 18S endogenous control gene imprinted on all TLDA plates, from the gene's cT value. Since cT values are logarithmic, this corresponds to dividing each gene's expression value by 18S's expression value. In other words, it is the fold change between a gene and 18S. Moving on with these values mean calculating fold change between groups based on genes' fold change with respect to 18S. dCt values are referred to as “scaled”.

Delta Ct Value Normalization

Once scaled, further normalization was done so that 12-gene valued vector for each sample has “length” or “amplitude” 1.

For a given sample, we calculated the “amplitude” or “length” of the 12 valued-vector (this is achieved by summing the square of each gene and then taking the square root) and then divide each gene value by this number.

Prediction Analysis

Following normalization, it was observed that 84 genes showed the same direction of expression in both TLDA and microarray results.

In the prediction analysis, we used the only genes in agreement between Affy and TLDA when genes that are “undetected” in 25 or more samples are filtered out. We found 84 genes to be detected and concordant between Affy and TLDA.

Leave-One-Out-Cross-Validation (L1OXV)

To arrive at the smallest, most predictive set from these 84 genes, Gema executed an iterative strategy called leave-one-out-cross-validation (L1OXV). L1OXV is explained as follows:

In this method, first number of genes in the predictive gene set, say P, is fixed. Then one sample in the training set is left-out and top P genes using the remaining samples that differentiate between N and F are calculated. Using these P genes the sample that is left out is predicted as N or F. This process is cycled through all 33 samples in the training set (leaving one out at a time). The total number of correct predictions is listed as the accuracy of the predictor on the training set.

During L1OXV process, different values for P, number of predictor genes, are tried and for ones that show good L1OXV prediction accuracy, these genes are applied on the validation set. The number of samples correctly predicted in the validation set is reported as prediction accuracy in the validation set. The smallest P that yields high training and validation accuracies are reported as the predictor gene set.

Prediction Analysis Results

Prediction analysis using these 84 confirmed genes and the normalized TLDA values of the 49 samples yielded a 12 gene signature with ˜72% prediction accuracy (35/49 correct predictions—14/24 N's; 21/25 F's correctly predicted). The predictor gene set remained significant using the Fisher's test, permutation test and randomization test (p-value <0.05).

The methods used to ascertain the 12 gene pregnancy signature are summarized below.

The first aspect of reducing the invention to practice involved identifying genes which constitute the pregnancy signature in women and potentially other mammals and was achieved by identifying and comparing the expression of genes in cumulus cells collected from women donors which are pregnancy competent or not. This was effected by collecting cumulus cells from different human oocytes of donor women and implanting patients with one or two putatively fertilized eggs. These patients were then, based on the results of the implantation, divided into three groups based on full, partial, and no pregnancy. For each oocyte used in the process, the transcriptional profile of at least one cumulus cell surrounding the particular oocyte was determined using Affymetrix HG 133 Plus 2 arrays containing over 54,000 transcripts. Patients were included in the study only if they did not meet any of the exclusion criteria identified in Table 1.

TABLE 1
Patient Exclusion Criteria

On Female Side:
>35 years of age
Low Ovarian Reserve
PCOS
> IVF cycle 2
Presence of >4 cm fibroids
BMI >35
History of chemotherapy of
radiation to abdomen or pelvis
On Male Side:
History of testicular biopsy
<5 million sperm

More particularly, in order to find gene signatures predictive of an oocyte's ability to produce a healthy baby, the inventors profiled the transcriptome of cumulus cells surrounding the oocyte using Affymetrix HG 133 Plus 2 arrays containing over 54,000 transcripts. Total RNA from individual cumulus samples was isolated using the PicoPure RNA isolation kit (Molecular Devices, Sunnyvale, Calif.). Sample RNA was amplified using a protocol developed in-house which ensures faithful and consistent amplification of small amounts of RNA to levels required for microarray analysis (Kocabas, et al., Proc Natl Acad Sci USA, 103, 14027-14032 (2006)).

Resulting amplified RNA (aRNA) was hybridized to the Affymetrix arrays. Thirty-six samples were used for which none of the embryo transfers led to successful pregnancies (labeled N for No success) and 30 samples for which all of the transfers led to successful pregnancies (labeled F for Full success). There were no known confounding factors to effect pregnancy success and relevant clinical parameters such as age or IVF cycle number did not vary significantly between the F and N groups.

Quality Control (QC) parameters were calculated for all 65 samples using Expression Console™ (EC) software freely available by the manufacturer (Affymetrix). All QC parameters including scaling factor (coefficient needed to equate the 2% trimmed mean of overall chip intensity), percentage of probe sets called present, 3′-5′ ratios for spike and labeling controls and housekeeping genes were within acceptable ranges (as described in manufacturer's guidelines) for all the samples. There were no known confounding factors to affect pregnancy success and relevant clinical parameters such as oocyte age or IVF cycle number did not vary significantly (t-test p>0.05) between F and N groups (see Table 1). Additional criteria for acceptance included absence of Polycystic Ovarian Syndrome (PCOS), no history of chemotherapy or radiation to the abdomen or pelvis, absence of >4 cm intramural or submucosal fibroids, and on the male side, no history of testicular biopsy and sperm count of >5 million.

In order to prove the soundness of the prediction model, F and N samples were divided randomly into training and validation sets. The goal was to find a predictive set of genes developed on the training set and then test the performance of the predictive genes on the validation set, which has not been used in development of the predictive model. This strategy (as opposed to using all the samples to develop a signature) prevents over-fitting and provides an assessment of predictive signature's robustness (Nevins, J. R. and Potti, A. (2007) Mining gene expression profiles: expression signatures as cancer phenotypes, Nat Rev Genet, 8, 601-609.)

A detailed summary of the materials and methods used to identify the preferred 12 gene “pregnancy signature” is provided below.

Materials and Methods Used to Identify 12-Gene Pregnancy Signature

Patient Selection, Implantation, and Pregnancy

This Institutional Review Board (IRB)-approved retrospective study included patients undergoing either IVF or ICSI from one clinical site in Chile, Clinica Las Condes (CLC) and from two in the U.S., Jarrett Fertility Group (JFG) and Pacific Fertility Center (PFC). One, two, or three embryos were transferred to each patient, and embryo transfers occurred on day 2, 3, or 5. Clinical pregnancy, defined as the presence of fetal heartbeat and gestational sac by first ultrasound examination, was determined between four and nine weeks following embryo transfer, depending upon the clinic's program. The Centers for Disease Control (CDC) use these as the standard criteria for defining pregnancy to report IVF results in the USA. This study included only samples from patients for whom all embryos transferred resulted in pregnancy (P, full success) or patients for whom zero embryos transferred resulted in pregnancy (N, no success). Live birth outcome was further recorded for patients with clinical pregnancy (P samples). We excluded patients older than 35, patients with fibroids larger than 4 cm in diameter, those with a body mass index greater than 35, or those with a history of chemo- or radiotherapy. Additionally, our study excluded families with severe male factor infertility as defined by a total sperm count of less than 5 million or a history of testicular biopsy.

Patient Stimulation

Clinicians determined the most appropriate means for stimulating their patients, but protocols generally combined either GnRH agonist or antagonist, to suppress spontaneous ovulation, with purified or recombinant FSH; they also either did or did not include hMG or luteal phase support. Ovarian response and follicular development were monitored by serum estradiol level and transvaginal ultrasound. We induced final follicular maturation by administering hCG and retrieved with ultrasound guidance 36 hours later.

Human CC Collection

Individual cumulus-oocyte-complexes (COCs) were rinsed in culture media to remove any blood, loose cells, or other debris. A small number of CCs from each COC, carefully were mechanically removed, careful to not take the very outer- or innermost layers. Each CC sample was rinsed in PBS and placed in a microcentrifuge tube with 100 μl, extraction buffer (Life Technologies, Carlsbad, Calif., USA) and resuspended gently by pipetting. Individual CC samples were incubated at 42° C. for 30 minutes, centrifuged, and frozen in liquid nitrogen until they were shipped to a processing laboratory. Corresponding oocytes were placed in individual culture drops and cultured individually until embryo transfer (ET).

RNA Isolation

RNA isolation was performed using the PicoPure RNA Isolation Kit (Life Technologies, Carlsbad, Calif., USA), according to the manufacturer's instructions. We analyzed total RNA quantity and quality using a NanoDrop 2000 spectrophotometer (NanoDrop Technologies, Wilmington, Del., USA). Total RNA isolation was done at Michigan State University, East Lansing, Mich., USA, and at GeneMarkers in Kalamazoo, Mich., USA.

Microarray Analysis

We performed transcriptional profiling of 64 individual CC samples (29 P, 35 N; Table 2) from 35 patients with Affymetrix HG-U 133 Plus 2.0 chips, which use more than 54,000 probe sets representing over 47,000 transcripts and variants. We synthesized and amplified cDNA using a protocol developed in house, as previously described (Kocabas A M, Crosby J, Ross P J, Otu H H, Beyhan Z, Can H et al. The transcriptome of human oocytes. Proc Natl Acad Sci USA 2006; 103:14027-32). Samples were analyzed with Affymetrix GeneChip Microarray Analysis Suite 5.0 and Expression Console software (Affymetrix Inc., Santa Clara, Calif., USA) for quality control assessment and normalization, following manufacturer's instructions.

Prediction Analysis

We applied the weighted voting approach utilizing “signal to noise ratio” (SNR) to assess predictor value of a gene g (Golub et al. 1999). Let μP(g) and μN(g) be the mean value of gene g in P and N sample groups, respectively. Similarly, let σP(g) and σN(g) be the standard deviation of gene g in P and N sample groups, respectively. SNR is defined as SNR(g)=[μF(g)−μN(g)]/[σF(g)+σN(g)]. This metric defines a neighborhood in RM around ideal gene expression vectors for both groups where M=|P|+|N|, total number of samples in the data set. SNR punishes genes with an expression highly deviant in either group and provides a signed ranking method for a gene's membership. In this case large positive values indicate a good predictor for the P group and large negative values (in absolute value) indicate a good predictor for the N group. The boundary between the idealized expression patterns and a given gene g is defined as B(g)=[μP(g)+μN(g)]/2.

When we are given a predictor gene set of T genes G={g1, g2, . . . , gT}, a group of P and N samples and a new sample S to be predicted. The vote of gi, 1≦i≦T, is defined as Vi=SNR(gi) [S(gi)−B(gi)], where S(gi) represents the signal value of gene gi in S. Vi represents how well S(gi) relates to the “behavior” of gi in P and N samples. If Vi is positive, we conclude that based on gi, S is predicted to be P and if Vi is negative gi predicts S as N. Cycling through all genes in the predictor set we obtain T votes used in the prediction of sample S.

When a prediction model is applied on a data set, the data set is first divided into Training and Validation sets. The predictor gene set is calculated on the Training set using leave-one-out cross-validation (L1OXV). In the L1OXV method utilizing a predictive gene set of T genes, one sample in the Training Set is left-out and top T genes using the remaining samples that differentiate between N and P are calculated. Using these T genes, the sample that is left out is predicted as N or F. This process is cycled through all samples in the Training Set leaving one out at a time. The total number of correct predictions is listed as the accuracy of the predictor on the training set. The predictor set of T genes is then applied on the Validation set. We assigned significance of the predictor genes using Fisher's test and two additional strategies: i) a permutation test, in which we randomly permuted class labels of P and N sample groups and identified optimum gene predictors using the same strategy ii) randomization test, in which we assessed the accuracy of T randomly chosen gene predictors using the original data set class labels. We compared the performance of the original predictor set with the results obtained using permutation and randomization tests to assess the original predictor set's significance. In both tests, we used 1000 realizations.

Quantitative Real-Time PCR

We performed cDNA synthesis using 8 ng total RNA with the High Capacity cDNA Reverse Transcription Kit (Life Technologies, Carlsbad, Calif., USA), according to the manufacturer's protocol. Preamplification was done according to the Taqman PreAmp Pools Protocol (Life Technologies) using a custom PreAmp Pool for 381 unique mRNA assays. Each sample reaction included 25 μL of 2× Taqman PreAmp Master Mix (Life Technologies), 12.5 μL of custom PreAmp Pool (Life Technologies), and 12.5 μL of cDNA template. The thermocycler conditions were as follows: 10 minutes at 95° C., followed by 14 cycles of 15 seconds at 95° C. and then 4 minutes at 60° C. We employed a custom Taqman Low Density Array (TLDA; Life Technologies) and ran one sample per array. Endogenous control genes 18S, GAPDH, and β-actin were included for relative quantification of transcripts. Forty-nine of the 64 individual CC samples previously used on microarray, along with 37 new individual biological CC samples from new patients, were analyzed on TLDA (Table 2).

Statistics

We used the GeNorm algorithm in Real-Time StatMiner (Integromics, Philadelphia, Pa., USA) software to identify the most stable endogenous control gene, or combination of endogenous control genes on the qRT-PCR TLDA across all sample sets. The Mann-Whitney test (Zar J H. Biostatistical Analysis (5th Edition). Upper Saddle River, N.J.: Pearson Prentice-Hall, 2010) was used to evaluate the clinical characteristics between pregnant (P) and nonpregnant (N) groups. Because we assessed several variables, we used α=0.01 to determine statistical significance so as to manage the potentially inflated false-positive error rate. Fisher's exact test was used to determine the significance of prediction results during the pregnancy prediction analysis of the qRT-PCR gene expression data. We employed analysis of variance (ANOVA) to assess categorical variable differences in gene expression, and we used Pearson's correlation to evaluate the relationship between continuous variables and gene expression. The ROC analysis was performed on the gene expression using the clinical pregnancy outcome (P, N) as the basis for truth. The ROC curve was created by plotting the true positive fraction (TPF or sensitivity) versus the false positive fraction (FPF or 1-specificity) determined by moving the cut-point value along the gene expression range. The area under this curve (AUC) indicates the degree of predictive ability of the gene expression ranging from 0.5 (random chance) to 1.0 (perfect). All analyses were carried out using SAS software (SAS V9.2; Cary, N.C., USA) or MedCalc (V11.3.1.0; Mariakerke, Belgium).

Results

Patient and Sample Clinical Characteristics

The analysis included a total of 101 CC samples, 86 of which were included on qRT-PCR TLDA from 55 patients (FIG. 1, Table 2). All TLDA P samples that were confirmed as clinical pregnancies at fetal heartbeat check advanced to healthy live birth.

Of the 86 samples used to confirm, refine, and validate the predictive gene set using qRT-PCR, 25, 45, and 16 samples were provided by CLC, JFG, and PFC, respectively (Table 5). The majority of samples came from double ETs (69), while eight CCs came from single ETs, and nine samples corresponded to triple ETs. ETs for 47 samples occurred on days 2/3, and 39 underwent ETs on day 5; no significant difference existed between P and N groups on the day of ET. We found no differences in the primary clinical characteristics, such as oocyte age and cycle number, between P and N groups (Table 7). However, we found a higher number of metaphase II (MID oocytes (p. 0.008) in the P group and a lower fertilization rate (number of 2PN from MII oocytes; p. 0.002) in the P group (Table 8). Due to these observed differences between groups, we ran a clinical correlate of gene expression analysis, which we describe in a later section.

Pregnancy Prediction Analysis

First, we used microarrays to obtain transcriptional profiling for 64 individual CC samples (35 N and 29 P; Table 2, FIG. 1). Signal-to-noise ratio (SNR) was used to assess the predictive value of a gene using weighted voting, as previously described (Golub T R, Slonim D K, Tamayo P, Huard C, Gaasenbeek M, Mesirov J P et al. Molecular classification of cancer: class discovery and class prediction by gene expression monitoring. Science 1999; 286:531-7). This group was divided into (1) a training set (18 N and 15 P) to find a predictive set of genes and (2) a validation set (17 N and 14 P). We used the validation set to test the performance of the predictive genes; the validation set comprised and consisted of samples that were not used in development of the predictive model. This strategy prevented overfitting and provided an assessment of the predictive signature's robustness (Nevins J R, Potti A. Mining gene expression profiles: expression signatures as cancer phenotypes. Nat Rev Genet 2007; 8:601-9). In order to find genes that correlated with success, we identified genes in the training set (P versus N) that showed differential expression based on t-tests (p<0.05 with Bonferroni correction for multiple hypothesis testing). The resulting 1180 genes, called “descriptive genes,” were used for L1OXV in the training set (Radmacher M D, McShane L M, Simon R. A paradigm for class prediction using gene expression profiles. J Comput Biol 2002; 9:505-11.). Weighted voting analysis revealed a 227 gene predictor set yielding 97% L1OXV accuracy (32/33 correct predictions—17/18 N and 15/15 P correctly predicted) on the training set and 87% (27/31 correct predictions—17/17 N and 10/14 P correctly predicted) prediction accuracy on the validation set. The prediction results remained significant using Fisher's test, the permutation test, and the randomization test (p<0.05).

Validation and Refinement of Predictive Genes with qRT-PCR

Of 227 genes found to be predictive of pregnancy outcome, we included 196 in our custom TLDA for qRT-PCR validation. The endogenous controls O-actin, GAPDH, and 18S were evaluated for the most stable expression across the sample set. We found that 18S alone was most stable, and Ct values were normalized to this gene's expression level, providing dCt values which represented the fold change of a sample's gene relative to 18S expression.

We used a subset of 49 samples (24 N and 25 P; Table 1, FIG. 1) out of 64 samples used in microarrays to confirm and further refine the predictive gene set. Following normalization to 185, we observed that 84 genes showed concordant expression on TLDA, as was previously determined on microarray with the same 49 biological samples. Using pregnancy prediction analysis on these 84 genes with the same strategy (weighted voting utilizing the SNR) yielded a predictive set of 12 genes. In order to further assess the predictive value of the 12-gene set, we ran TLDA on 37 new biological samples from new patients (19 N and 18 P; Table 1, FIG. 1) not used in the microarray analysis. The predictor gene set remained significant using Fisher's test, the permutation test, and the randomization test (p<0.05) during both refinement and validation procedures.

Gene Expression in Cumulus Cells as a Biomarker of Pregnancy Outcome

The 12-gene predictor set identified using qRT-PCR TLDA on Sample Set A′ (49 samples previously screened by microarray) was validated on Sample Set B (37 new biological samples not used by microarray) using weighted voting as previously described. Seven genes were upregulated in P samples compared to N, and five genes were downregulated in P compared to N group (Table 5). When applied to the validating B data set (37 samples), this pregnancy prediction model yielded an accuracy of 78%, a sensitivity for identifying successful pregnancy outcomes of 72%, a specificity for identifying failed pregnancy outcomes of 84%, a positive predictive value (PPV) of 81%, and a negative predictive value (NPV) of 76% (Table 3).

Receiver Operating Characteristic (ROC) analysis, a common method for evaluating the diagnostic utility of a test (Zhou K H, O'Malley A J, Mauri L. Receiver-operating characteristic analysis for evaluating diagnostic tests and predictive models. Circulation 2007; 115:654-7; and Linden A. Measuring diagnostic and predictive accuracy in disease management: an introduction to receiver operating characteristic (ROC) analysis. J Eval Clin Pract 2006; 12:132-9;), was conducted to determine the predictive power of identifying a successful pregnancy outcome based upon the 12-gene prediction values for the validating 37 B samples (Table 4, FIG. 2). The AUC, which indicates the degree of predictive ability, was 0.763±0.079, which is significantly (p=0.0009) greater than 0.5 (random chance prediction). Our sample size and the AUC observed in our ROC analysis fall in line with previous diagnostic reports within the IVF field (Esterhuizen A D, Franken D R, Lourens J G H, Prinsloo E, van Rooyen L H. Sperm chromatin packaging as an indicator of in-vitro fertilization rates. Hum Reprod 2000; 15:657-61; and Fabregues F, Balasch J, Creus M, Carmona F, Puerto B, Quinto L et al. Ovarian Reserve Test with Human Menopausal Gonadotropin as a Predictor of In Vitro Fertilization Outcome. J Assist Reprod Genet 2000; 17:13-9).

Clinical Correlates of Gene Expression

We evaluated patients' clinical characteristics for potential correlation with the 12-gene expression prediction values. Again, because several variables were being assessed, we used α=0.01 to determine statistical significance to manage the potentially inflated false-positive error rate. Of the continuous variables, none significantly correlated with the prediction value (Table 8), including the number of MII oocytes and the fertilization rate (2PN/MII), despite their displaying different values between pregnant and nonpregnant samples. Although the number of MII oocytes and the fertilization rate differed significantly in the pregnancy outcome groups, neither variable correlated with the gene expression signature. That is, despite different numbers of MIT oocytes and different fertilization rates between P and N groups, this did not seem to affect the strength of the pregnancy signature.

The differences in the sum of the 12-gene prediction value for the categorical assessments were evaluated using ANOVA. If the overall test for category differences was considered significant at α=0.01, then we evaluated pairwise comparisons of the categories. Only two categorical variables, gonadotropin and ET catheter, were found to differ significantly in gene expression (Table 9). Regarding gonadotropin, only JFG used the pFSH/hMG regimen (n=45); PFC used rFSH exclusively (n=16). Thus, we found a degree of confounding between site and gonadotropin, and these results should be interpreted with caution. Similarly, regarding the ET catheter, results should be interpreted cautiously, as a confounding effect resulted from each site using different catheters exclusively. Further, the Wallace catheter sample size was very small (n=5), providing very little power from which to draw conclusions. Finally, with respect to clinical site, the majority of samples from CLC were collected much earlier and stored longer than those from JFG, likely explaining the difference seen in predictive values between these sites.

Tables 2-9 referenced supra are set forth below.

### Tables

TABLE 2
Patient and sample numbers by sample set and platform
Samples (Patients)

| Set A - Array* | | | | | | | |
|---|---|---|---|---|---|---|---|
| n = 64 (35)^{†} | | | | Set A′ - qPCR** | | Set B - qPCR*** | |
| Training | | Validation | | n = 49 (33) | | n = 37 (22) | |
| P | N | P | N | P | N | P | N |
| 15 | 18 | 14 | 17 | 25 | 24 | 18 | 19 |
| (14) | (16) | (12) | (15) | (16) | (17) | (11) | (11) |

P = Pregnant samples; N = Non Pregnant Samples
*Set A: 64 samples first used on array to identify first set of 227 predictive genes
**Set A′: 49 samples (from the 64) used on qPCR TLDA to confirm and refine to 12 predictive genes
***Set B: 37 new biological samples used on qPCR TLDA to validate final 12-gene predictive set
^{†}Most patients contributed sibling samples to both the Training and Validation Sets

TABLE 3
Specific predictive accuracies of the 12-gene pregnancy
signature on validating B sample set*

| | | |
|---|---|---|
| Overall Accuracy | 78% | (29/37) |
| Sensitivity | 72% | (13/18) |
| Specificity | 84% | (16/19) |
| Positive Predictive Value | 81% | (13/19) |
| Negative Predictive Value | 76% | (16/18) |
| Odds Ratio for Successful Outcome | 13.9 | (2.8, 69.2) |
| (95% CI) | | |

| | |
|---|---|
| p (OR = 1) | 0.0006 |

*Percentages refer to number of fetal heartbeats over number of embryos transferred

TABLE 4
Predictive power of the 12-gene pregnancy signature*

| | Combined | | |
|---|---|---|---|
| | A′ + B | | Validating |
| | Sample Sets | Sample Set A′ | Sample Set B |
| #Successes/#Failures | 43/43 | 25/24 | 18/19 |
| AUC ± Standard Error | 0.725 ± 0.055 | 0.703 ± 0.075 | 0.763 ± 0.079 |
| 95% Confidence | 0.618, 0.816 | 0.556, 0.825 | 0.595, 0.887 |
| Interval | | | |
| Prob (AUC = 0.5)** | <0.0001 | 0.0067 | 0.0009 |
| Sensitivity at | 65% | 56% | 72% |
| Threshold | | | |
| Specificity at | 77% | 79% | 84% |
| Threshold | | | |

AUC = Area Under the Curve
**Degree of predictive ability (p-value), significantly greater than 0.5, random chance prediction
*Percentages refer to number of fetal heartbeats over number of embryos transferred

TABLE 5
qRT-PCR patient and sample numbers by clinic

| | Samples (Patients) | | |
|---|---|---|---|
| | n = 55 (86) | | |
| | P | N | Total |
| CLC | 8 (14) | 11 (8) | 25 (16) |
| JFG | 20 (12) | 25 (15) | 45 (27) |
| PFC | 9 (7) | 7 (5) | 16 (12) |
| Total | 43 (27) | 43 (28) | 86 (55) |

P = Pregnant samples;
N = Non Pregnant samples

TABLE 6
qRT-PCR sample clinical characteristics

| | | P (Pregnant) | | N (Non Pregnant) | | |
|---|---|---|---|---|---|---|
| | | n = 43 | | n = 43 | | |
| Variable | Unit | Average | SD | Average | SD | p |
| Oocyte Age | Year | 31.26 | 0.50 | 29.53 | 0.63 | 0.675 |
| BMI | kg/m² | 23.27 | 0.58 | 23.38 | 0.56 | 0.572 |
| IVF Cycle | # | 1.44 | 0.13 | 1.37 | 0.07 | 0.573 |
| # Oocytes ER | # | 12.74 | 1.15 | 10.44 | 0.95 | 0.156 |
| MII Oocytes | # | 10.16 | 0.94 | 7.23 | 0.76 | 0.008* |
| Oocyte Maturity | % | 82.46 | 3.67 | 74.37 | 4.19 | 0.149 |
| 2PN | # | 7.40 | 0.66 | 5.72 | 0.59 | 0.056 |
| Fertilization | % | 61.86 | 3.46 | 60.76 | 4.03 | 0.856 |
| Rate** | | | | | | |
| (2PN/ER#) | | | | | | |
| Fertilization | % | 74.54 | 2.30 | 83.92 | 3.11 | 0.002* |
| Rate** | | | | | | |
| (2PN/MII Insem.) | | | | | | |
| Day of ET | # | 3.91 | 0.18 | 3.63 | 0.18 | 0.276 |

*Indicates significant difference between P and N groups
**Statistics were run after first calculating the rates for each patient individually
# Oocytes ER = Number of oocytes retrieved

TABLE 7
Set of 12 genes used to predict pregnancy outcome

| Gene | | P over N | |
|---|---|---|---|
| Symbol | Gene Name | (Fold Change) | Known or Suggested Function* |
| FGF12 | Fibroblast growth | Up (1.52) | FGF family involved in an array of biological |
| | factor 12 | | processes including cell growth, morphogenesis, |
| | | | embryonic development, and tissue repair. |
| GPR137B | G-coupled protein | Up (1.31) | G-protein coupled receptor (GPCR) family are |
| | receptor 13b | | integral membrane proteins, and play a prominent |
| | | | role in interpreting external messages for a cell |
| | | | and inducing signaling cascades within the cell. |
| SLC2A9 | Solute carrier family | Up (1.26) | The SLC2A family plays significant role in |
| | 2 (facilitated glucose | | maintaining glucose homeostasis. This gene |
| | transporter), member 9 | | facilitates glucose transport. |
| ARID1B | AT rich interactive | Up (1.57) | Chromatin remodeling-dependent transcriptional |
| | domain 1B (SWI1- | | regulation. |
| | like) | | |
| NR2F6 | Nuclear receptor | Up (1.15) | Inhibits human luteinizing hormone receptor (hLHr) |
| | subfamily 2, group F, | | transcription. |
| | member 6 | | |
| ZNF132 | Zinc finger protein | Up (1.08) | Zing finger proteins assist in directly affecting |
| | 132 | | transcription by conferring DNA sequence |
| | | | specificity as the DNA-binding domain of multi- |
| | | | subunit transcription factors. |
| FAM36A | Family with | Up (1.32) | Unknown function but integral membrane and |
| | sequence similarity | | mitochondrial localization. |
| | 36, member A | | |
| ZNF93 | Zinc finger protein 93 | Down (−1.62) | Zing finger proteins assist in directly affecting |
| | | | transcription by conferring DNA sequence |
| | | | specificity as the DNA-binding domain of multi- |
| | | | subunit transcription factors. |
| RHBDL2 | Rhomboid, veinlike 2 | Down (−1.11) | An intermembrane protease; intermembrane |
| | (Drosophila) | | proteolysis is progressively being more recognized |
| | | | as participating in regulation of a host of cellular |
| | | | processes such as development and metabolism. |
| DNAJC15 | DnaJ (Hsp40) | Down (−6.52) | Localized to mitochondria membrane, and |
| | homolog, subfamily | | thought to have heat shock binding properties. |
| | C, member 15 | | |
| MTUS1 | Microtubule | Down (−1.42) | Identified as highly expressed in ovary relative to |
| | associated tumor | | other tissues, but its function in this region in |
| | suppressor 1 | | unknown. |
| NUP133 | Nucleoporin 133 kDa | Down (−1.28) | Nucleocytoplasmic transport activity. |

*http://www.ncbi.nlm.nih.gov/gene/

TABLE 8
Continuous variable correlation with
prediction value

| | Correlation | p (Corr = 0) |
|---|---|---|
| Oocyte Age | −0.14 | 0.1986 |
| BMI | −0.09 | 0.4532 |
| # Follicles | 0.06 | 0.5640 |
| # Oocytes ER (#ER) | −0.07 | 0.5444 |
| # Mature Oocytes (MII) | −0.15 | 0.1600 |
| # Oocytes Fertilized (2PN) | −0.14 | 0.2016 |
| Fertilization Rate | −0.10 | 0.3361 |
| (2PN/#ER) | | |
| Fertilization Rate (2PN/MII) | 0.07 | 0.5228 |

# Oocytes ER = Number of oocytes retrieved

TABLE 9
Categorical variable correlation with prediction value

| | p-value for | | |
|---|---|---|---|
| | Overall | | |
| | Differences | Significant Pairwise Comparisons | |
| | from ANOVA | (n) | |
| Site | 0.0133 | CLC (25) vs JFG (45) | p = 0.0034 |
| GnRH Analog | 0.0970 | | |
| Gonadotropin | 0.0030* | pFSH/hMG (28) vs rFSH (19) | p = 0.0081 |
| | | pFSH/hMG (28) vs rFSH/hMG (39) | p = 0.0014 |
| Fertilization | 0.3605 | | |
| ET Catheter | 0.0016* | Wallace (5) vs Frydman (13) | p = 0.0010 |
| | | Wallace (5) vs Cook (11) | p = 0.0152 |
| | | Wallace (5) vs Soft-echo (12) | p = 0.0426 |
| | | USP (46) vs Frydman (13) | p = 0.0006 |
| Luteal-Phase | 0.4261 | | |
| ET Day | 0.0235 | | |
| IVF Cycle | 0.1367 | | |
| # Embryos ET | 0.0361 | | |

*Indicates significant difference between P and N groups
pFSH = purified FSH;
rFSH = recombinant FSH

### DISCUSSION

The ability to select viable oocytes and embryos during IVF has significant medical, social, and financial benefits. A diagnostic assay using CCs that complements morphology would present a noninvasive approach to attaining this goal. A critical question, however, has remained whether developing a test robust enough to overcome inherent variations in patients and clinics would be possible. This report describes, for the first time, a novel set of 12 genes—produced from multiple sites and diverse clinical protocols—that predict pregnancy outcome. Our proposed prediction strategy, based on the expression levels of the genes in CCs, paves the way for a noninvasive supplementary tool for selecting viable oocytes. We developed the predictive gene set using a global expression profiling approach and then employed qRT-PCR to validate it on two independent biological sample sets. Additional ROC analysis confirmed that this predictive gene set has significant predictive power.

While the genes that ultimately comprised our final gene set do not overlap with genes reported as predictive of pregnancy previously, this is not entirely surprising. This could be due to several factors: differences in technical approaches such as the use of TLDAs, the fact that our algorithm incorporates weighted voting which places varied contribution of each gene's expression in the prediction model, or a combination of both.

The genes in our predictive set are, in part, involved with glucose metabolism, transcriptional regulation, gonadotropin regulation, and apoptosis—all essential to viable COC processes. Considering the generally known functions of some of the genes or gene families, it is not improbable that they could reveal themselves as part of a pregnancy predictive CC gene panel. For example, since the fibroblast growth factor (FGF) family plays an important role in regulating cell survival, FGF12 appears upregulated in our P group compared to the N group of samples.

Glucose, which is metabolized by the glycolysis pathway, acts as a crucial metabolite for the COC (Leese H J, Baumann C G, Brison D R, McEvoy T G, Sturmey R G. Metabolism of the viable mammalian embryo: quietness revisited. Mol Hum Reprod 2008; 14:667-72.). The breakdown of glucose by CCs provides the oocyte with essential nutrients, such as pyruvate and lactate, to complete maturation in preparation for ovulation. Converting glucose into these byproducts has further importance: providing the oocyte with the maternal store of metabolites/energy sources as it is nurtured by the surrounding granulosa cells, of which CCs are one type. Thus, granulosa cells play a critical role in supporting the developing oocyte and establishing its maternal supply of energy resources to carry it through the first few cell divisions (Watson A J. Oocyte cytoplasmic maturation: A key mediator of oocyte and embryo developmental competence. J Anim Sci 2007; 85:E1-E3.). SCL2A9 (also known as GLUT9), a member of the SLC2A facilitative transporter family, plays an important role in glucose homeostasis (Sutton-McDowall M L, Gilchrist R B, Thompson J G. The pivotal role of glucose metabolism in determining oocyte developmental competence. Reproduction 2010; 139:685-95). Specifically, SCL2A9 has been demonstrated to transport uric acid and hexose sugars, of which glucose is one example (Augustin R, Carayannopoulos M O, Dowd L O, Phay J E, Moley J F, Moley K H. Identification and characterization of human glucose transporter-like protein-9 (GLUT9): alternative splicing alters trafficking. J Biol Chem 2004; 279:16229-36). In the bovine model, mature COCs were observed to utilize more glucose and its metabolic products than immature COCs (Sutton M L, Cetica P D, Beconi M T, Kind K L, Gilchrist R B, Thompson J G. Influence of oocyte-secreted factors and culture duration on the metabolic activity of bovine cumulus cell complexes. Reproduction 2003; 126:27-34). Given this fact, the increased expression of SCL2A9 in CCs corresponding to viable oocytes may reflect a more dynamic transport of glucose within those CCs and therefore a more properly functioning metabolic state in these COCs as a whole.

NR2F6 was also upregulated in our P sample sets relative to N. This gene is an orphan nuclear receptor, belonging to a subgroup of the nuclear receptor superfamily of transcription factors and cofactors. While the exact function of NR2F6 remains undefined in CCs, orphan nuclear receptors are known to play a role in many reproductive processes (Bertolin K, Bellefleur A-M, Zhang C, Murphy B D. Orphan nuclear receptor regulation of reproduction. Animal Reproduction 2010; 7:146-53). Specifically, research has shown that NR2F6 inhibits luteinizing hormone receptor (LHr) transcription via promoter repression (Zhang Y, Dufau M L. Nuclear orphan receptors regulate transcription of the gene for the human luteinizing hormone receptor. J Biol Chem 2000; 275:2763-70;). The formation of LHr on the surface of CCs plays a key part in proper follicular maturation prior to the LH surge, which induces ovulation. However, overexpression of LHr can also have adverse effects on the ovulatory process, as higher levels of this receptor have been reported in the granulosa cells of women with polycystic ovaries compared to those without (Jakimiuk A J, Weitsman S R, Navab A, Magoffin D A. Luteinizing Hormone Receptor, Steroidogenesis Acute Regulatory Protein, and Steroidogenic Enzyme Messenger Ribonucleic Acids Are Overexpressed in Thecal and Granulosa Cells from Polycystic Ovaries. J Clin Endocrinol Metab 2001; 86:1318-23). The slightly lower expression of NR2F6 seen in our N group may indicate a hyperactive state of LHr expression, which could lead to suboptimal maturation of the follicle.

We found four additional genes that were upregulated in the CCs of P samples compared to N samples: ARID1B, FAM36A, GPR137B, and ZNF132. ARID1B is part of the SWI/SNF chromatin remodeling complex, which plays a critical role in cell cycle control. Research has demonstrated the necessity of open gap junction communication between follicular cells and their oocyte for proper meiotic maturation, which involves chromatin remodeling maturation (Luciano A M, Franciosi F, Modina S C, Lodde V. Gap Junction-Mediated Communications Regulate Chromatin Remodeling During Bovine Oocyte Growth and Differentiation Through cAMP-Dependent Mechanism(s). Biol Reprod 2011; 85:1252-9). Increased ARID1B in our P samples may facilitate gap junction communication and improve oocyte viability. The function of FAM36A is not well characterized, but this protein has been localized in mitochondria and is integral to the membrane. GPR137B is also poorly characterized; however, this gene encodes a G-protein-coupled receptor (GPCR) integral membrane protein. Given the prominent role GPCRs play in interpreting external messages for a cell, this could indicate an important role for GPR137B in signaling within the follicular microenvironment. ZNF132—yet another gene with a poorly understood function—is, however, a member of the zinc finger protein family, which aids in directly affecting transcription by acting as the DNA-binding subunit of transcription factors, thus conferring DNA sequence specificity.

Five genes in our signature were downregulated in P versus N samples: DNAJC15, RHBDL2, MTUS1, NUP133, and ZNF93. Little is known about the specific action of these genes. DNAJC15 is localized to mitochondria and membranes and is thought to have heat-shock-binding properties. RHBDL2 is an intermembrane protease, and research increasingly suggests the importance of intermembrane proteolysis in regulating a variety of cellular processes, such as development and metabolism (Erez E, Fass D, Bibi E. How intramembrane proteases bury hydrolytic reactions in the membrane. Nature 2009; 459:371-8). MTUS1 has previously been reported as more highly expressed in ovaries than in other tissues (Nagase T, Ishikawa K-i, Kikuno R, Hirosawa M, Nomura N, Ohara O. Prediction of the Coding Sequences of Unidentified Human Genes. XV. The Complete Sequences of 100 New cDNA Clones from Brain Which Code for Large Proteins in vitro. DNA Research 1999; 6:337-45; Nagase T, Ishikawa K-i, Kikuno R, Hirosawa M, Nomura N, Ohara O. Prediction of the Coding Sequences of Unidentified Human Genes. XV. The Complete Sequences of 100 New cDNA Clones from Brain Which Code for Large Proteins in vitro. DNA Research 1999; 6:337-45)), although the specific action of this gene in ovarian regions remains documented. NUP133 is involved with nucleocytoplasmic transport activity, a subset of which includes glucose transport. Finally, ZNF93, another zinc finger gene, has an as-yet-undescribed function but is thought, like other characterized zinc finger proteins, to regulate transcription in a direct manner as the DNA-binding component of transcription factors.

The functional role of each gene in our predictive set with respect to oocyte and embryo viability remains to be elucidated. Hypothesis-driven experiments are required to interrogate how each gene expressed in CCs acts individually, and in combination, to impart or compromise the developmental competence of their respective oocyte, dependent on its level of expression.

Despite a significant difference in the number of MII oocytes and the fertilization rate between samples from pregnant and nonpregnant patients, the clinical correlates of gene expression analysis has demonstrated that these differences have no correlation with the gene expression values, and therefore no effect on the strength of our predictive gene set.

The effect on gene expression values identified in gonadotropin choice and ET catheter between pregnancy outcome groups appears more indicative of the clinical site, as usage of these factors were confounded with site. Again, regarding the clinical site difference seen between CLC and JFG, the majority of samples from CLC were collected earlier and stored longer than those from the JFG, likely explaining the difference seen in this covariate.

The data presented herein reveal a novel 12-gene set in CCs that are predictive of pregnancy; these data, from multiple sites using multiple stimulation protocols, had an overall accuracy of 78%. ROC analysis confirms the predictive power of our test, with an AUC=0.763±0.079, which is significantly greater than the 0.5 of random chance prediction (p=0.0009) and comparable with the expectation for a successful diagnostic test. This is particularly promising given the heterogeneous nature of the patients and the treatment differences in the treatment they received.

This gene signature may be applied to randomized control clinical trial across multiple sites in order to further confirm its pregnancy prediction value in identifying the oocytes with the highest pregnancy potential for embryo transfer.

In conclusion, using accepted statistical methods the inventors identified 12 genes, i.e., FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1), wherein the levels of expression of one of these genes, or any combination of these genes of by cumulus cells correlates to the capability of an oocyte associated therewith or from the same women donor to result in a viable pregnancy. Therefore, methods which detect the expression of one or more of these 12 genes by a cumulus cell may be used in order to determine whether an oocyte associated therewith or from the same women donor is suitable for use in an IVF procedure, as well as for identifying individuals with conditions that result in oocytes unsuitable for use in IVF procedures, and for monitoring the success of fertility treatments.

TABLE 10
Optimal 12 Gene Preganancy Signature Set and Gene
Accession Numbers

| Assay No | Gene Symbol |
|---|---|
| Hs00374427_m1 | FGF12 |
| Hs00162803_m1 | GPR137B |
| Hs00417125_m1 | SLC2A9 |
| Hs00368175_m1 | ARID1B |
| Hs00172870_m1 | NR2F6 |
| Hs01036387_m1 | ZNF132 |
| Hs00831105_s1 | FAM36A |
| Hs01656246_s1 | ZNF93 |
| Hs00384848_m1 | RHBDL2 |
| Hs00387763_m1 | DNAJC15 |
| Hs00826834_m1 | MTUS1 |
| Hs00217272_m1 | NUP133 |

Throughout this application, various references describe the state of the art to which this invention pertains. The disclosures of these references are hereby incorporated by reference into the present disclosure.

| Sequence Listing Containing Exemplary Polypeptide and Nucleic Acid | |
|---|---|
| Sequences for 12 Pregnancy Signature Genes | |
| 1. FGF12 Gene | |
| A. Human FGF-12 Polypeptide Sequence | |
| (SEQ ID NO: 1) | |
| MESKEPQLKGIVTRLFSQQGYFLQMHPDGTIDGTKDENSDYTLFNLIP | |
| VGLRVVAIQGVKASLYVAMNGEGYLYSSDVFTPECKFKESVFENYYVIYSSTL | |
| YRQQESGRAWFLGLNKEGQIMKGNRVKKTKPSSHFVPKPIEVCMYREQSLH | |
| EIGEKQGRSRKSSGTPTMNGGKVVNQDST | |
| B. Human FGF-12 Nucleic Acid Sequence (mRNA coding sequence) | |
| (SEQ ID NO: 2) | |
| 1 | aaatctgctg tgcatccaga gagcaaagtg ggatgatctg tcactacacc tgcagcacca |
| 61 | cgctcggagg acagctcctg cctgcagctt ccagacccag gaagcctgag gggaaggaag |
| 121 | gaagtacggg cgaaatcatc agattggctt cccagatttg ggaatctgaa gcgggcccac |
| 181 | atcttccggc caacttccat tgaacttccc agcactcgaa agggaccgaa atggagagca |
| 241 | aagaacccca gctaaaaggg attgtgacaa ggttattcag ccagcaggga tacttcctgc |
| 301 | agatgcaccc agatggtacc attgatggga ccaaggacga aaacagcgac tacactctct |
| 361 | tcaatctaat tcccgtgggc ctgcgtgtag tggccatcca aggagtgaag gctagcctct |
| 421 | atgtggccat gaatggtgaa ggctatctct acagttcaga tgttttcact ccagaatgca |
| 481 | aattcaagga atctgtgttt gaaaactact atgtgatcta ttcttccaca ctgtaccgcc |
| 541 | agcaagaatc aggccgagct tggtttctgg gactcaataa agaaggtcaa attatgaagg |
| 601 | ggaacagagt gaagaaaacc aagccctcat cacattttgt accgaaacct attgaagtgt |
| 661 | gtatgtacag agaacaatcg ctacatgaaa ttggagaaaa acaagggcgt tcaaggaaaa |
| 721 | gttctggaac accaaccatg aatggaggca aagttgtgaa tcaagattca acatagctga |
| 781 | gaactctccc cttcttccct ctctcatccc ttccccttcc cttccttccc atttacccat |
| 841 | ttccttccag taaatccacc caaggagagg aaaataaaat gacaacgcaa gacctagtgg |
| 901 | ctaagattct gcactcaaaa tcttcctttg tgtaggacaa gaaaattgaa ccaaagcttg |
| 961 | cttgttgcaa tgtggtagaa aattcacgtg cacaaagatt agcacactta aaagcaaagg |
| 1021 | aaaaaataaa tcagaactca ataaatatta aactaaactg tattgttatt agtagaaggc |
| 1081 | taattgtaat gaagacatta ataaagatga aataaactta ttactttaaa ggaaaggatt |
| 1141 | tggagaattg aactcacaaa ctgatgttat atactcaata gcttaaactc atgataatgc |
| 1201 | tgcgatgtgt ggttttgctt gattttgtat tttatttggg catctggaat tgacacacca |
| 1261 | ttacattctg tttgcaggat tttttttgta accatgaaat tgaacatttc caaattataa |
| 1321 | actatgttaa tacctataaa atatatagcc aggaaccatt tatcatcaag aaaagtgtaa |
| 1381 | gaaattattt ttgagatgta atttaagatt gttttatgta aaaggaaaat cttgtatggc |
| 1441 | atcgaatagc cttaatgaat ttaattcttt cacaaaaatg atttcaaatt atcctagagt |
| 1501 | ataacatttt tatcaaagat attatttccg gagttcttct ttctttcttt tttttttttt |
| 1561 | tttagtaatt tagcaaaaac attactgttc taatgctgaa gtgacttttg ccagtgccat |
| 1621 | gtccaggtgg tgaggtataa gttacttgct cttagcattt ggtctgattt ttttgctttg |
| 1681 | tggacacctt tgagagtatc cacaaagcaa tgtctcaggt gtggacacct gagagcatgt |
| 1741 | tttagaaagc tttgtaccct gtcttgtggc aggaaagaaa gaacaggggt tttacataag |
| 1801 | gaaataagtc ctaggaaatt agtcaacgca aattgcattt gcctttgtac cttaccacag |
| 1861 | tcttatattg ttttttaaac tctgccatga aatttggaga catgactgtg aaattcctaa |
| 1921 | cttactatct tacaaagcca gtagctaatt tgttgctcta tgtatgatcc tgttacaagt |
| 1981 | ccagtttgca attcatttgt ttcctagaac acagaagggt accagtaata cactaaatgt |
| 2041 | tcaaggtgtg tagagaaata atatggaatt agcagctatg actccaacag acaggattgt |
| 2101 | gtgagcagct gaaaggagca aaaaagaact cagtgtaaga gaaggcacat acatagttaa |
| 2161 | gaatactaaa gtatttttaa aaatcaagga agaaataaat gttacacaat ttgcattgga |
| 2221 | ataaatagat ctatttagtc ctacaaatca ggagtggtgt agagacatcc aaatttaaag |
| 2281 | aaaaaaaaac acaaaacaga atgttaaaaa tgtatgcaga tttatggata ttatcaatga |
| 2341 | gaagacatag catgtaactt ctcctatatc tctactgtcc agcatgtatt gttccaaata |
| 2401 | tgactcccta aaatatatac actttgcaga agctctaggc cctcacctca aaccttgcca |
| 2461 | ttggttgccg tatttcaagg tcaatatagt ttccctcact ttacacaatc attattcttc |
| 2521 | aatagtggac catatccttc accaggtatc ctatttctgt tatctagagg ttagcagaaa |
| 2581 | atgaaatgaa ggaatttccc taagcagttg ggaagaacaa attgtatgca tgtaggcaaa |
| 2641 | gattttgaag atacatttgc aagagatatt tgtttaacca aaatatttgg aaagtaacaa |
| 2701 | ataaagacat ttaaattttc taaaaaaaaa aaaaaaaaca aaaaaaaaaa aaaa |
| 2. GP137B Gene | |
| A. Human GPR137B Polypeptide Sequence | |
| (SEQ ID NO: 3) | |
| MRPERPRPRGSAPGPMETPPWDPARNDSLPPTLTPAVPPYVKLGLTVVYTVF | |
| YALLFVFIYVQLWLVLRYRHKRLSYQSVFLFLCLFWASLRTVLFSFYFKDFVA | |
| ANSLSPFVFWLLYCFPVCLQFFTLTLMNLYFTQVIFKAKSKYSPELLKYRLPL | |
| YLASLFISLVFLLVNLTCAVLVKTGNWERKVIVSVRVAINDTLFVLCAVSLSIC | |
| LYKISKMSLANIYLESKGSSVCQVTAIGVTVILLYTSRACYNLFILSFSQNKSV | |
| HSFDYDWYNVSDQADLKNQLGDAGYVLFGVVLFVWELLPTTLVVYFFRVRN | |
| PTKDLTNPGMVPSHGFSPRSYFFDNPRRYDSDDDLAWNIAPQGLQGGFAPD | |
| YYDWGQQTNSFLAQAGTLQDSTLDPDKPSLG | |
| B. Human GPR137B Nucleic Acid Sequence | |
| (SEQ ID NO: 4) | |
| 1 | gcggcttgtt ttctttcctc cagtctcggg gctgcaggct gagcgcgatg cgcggagacc |
| 61 | cccgcggggg cggcggcggc cgtgagcccc gatgaggccc gagcgtcccc ggccgcgcgg |
| 121 | cagcgccccc ggcccgatgg agaccccgcc gtgggaccca gcccgcaacg actcgctgcc |
| 181 | gcccacgctg accccggccg tgccccccta cgtgaagctt ggcctcaccg tcgtctacac |
| 241 | cgtgttctac gcgctgctct tcgtgttcat ctacgtgcag ctctggctgg tgctgcgtta |
| 301 | ccgccacaag cggctcagct accagagcgt cttcctcttt ctctgcctct tctgggcctc |
| 361 | cctgcggacc gtcctcttct ccttctactt caaagacttc gtggcggcca attcgctcag |
| 421 | ccccttcgtc ttctggctgc tctactgctt ccctgtgtgc ctgcagtttt tcaccctcac |
| 481 | gctgatgaac ttgtacttca cgcaggtgat tttcaaagcc aagtcaaaat attctccaga |
| 541 | attactcaaa taccggttgc ccctctacct ggcctccctc ttcatcagcc ttgttttcct |
| 601 | gttggtgaat ttaacctgtg ctgtgctggt aaagacggga aattgggaga ggaaggttat |
| 661 | cgtctctgtg cgagtggcca ttaatgacac gctcttcgtg ctgtgtgccg tctctctctc |
| 721 | catctgtctc tacaaaatct ctaagatgtc cttagccaac atttacttgg agtccaaggg |
| 781 | ctcctccgtg tgtcaagtga ctgccatcgg tgtcaccgtg atactgcttt acacctctcg |
| 841 | ggcctgctac aacctgttca tcctgtcatt ttctcagaac aagagcgtcc attcctttga |
| 901 | ttatgactgg tacaatgtat cagaccaggc agatttgaag aatcagctgg gagatgctgg |
| 961 | atacgtatta tttggagtgg tgttatttgt ttgggaactc ttacctacca ccttagtcgt |
| 1021 | ttatttcttc cgagttagaa atcctacaaa ggaccttacc aaccctggaa tggtccccag |
| 1081 | ccatggattc agtcccagat cttatttctt tgacaaccct cgaagatatg acagtgatga |
| 1141 | tgaccttgcc tggaacattg cccctcaggg acttcaggga ggttttgctc cagattacta |
| 1201 | tgattgggga caacaaacta acagcttcct ggcacaagca ggaactttgc aagactcaac |
| 1261 | tttggatcct gacaaaccaa gccttgggta gcatcagtta acagttttat ggacgattcc |
| 1321 | tcagatgaaa agcttcagaa aagcatagtg acagctgaat ttttagggca cttttcctta |
| 1381 | agaaatagaa cttgattttt atttgttaca ggtttccaat ggccccatag gaataagcaa |
| 1441 | taatgtagac tgataaaccc ttattttagt actaaagagg gagccttgct atttcagtgg |
| 1501 | gtataattta aactttttaa agaaaatctg tacttttata aagatgtatt ttgtataact |
| 1561 | taaataataa tgctaaagta tactagggtt tttttttctt gagaatgtta ctgcaatcat |
| 1621 | gttgtagttt gcacagactt ttatgcataa ttcactttaa aaatatagaa tatatggtct |
| 1681 | aatagttaaa aaaaaaaaaa aaaaa |
| 3. GLUT9 (SLC2A9) Gene | |
| A. Human GLUT9 (SLC2A9) Polypeptide Sequence | |
| (SEQ ID NO: 5) | |
| MARKQNRNSKELGLVPLTDDTSHARPPGPGRALLECDHLRSGVPGGRRRKD | |
| WSCSLLVASLAGAFGSSFLYGYNLSVVNAPTPYIKAFYNESWERRHGRPIDPD | |
| TLTLLWSVTVSIFAIGGLVGTLIVKMIGKVLGRKHTLLANNGFAISAALLMACS | |
| LQAGAFEMLIVGRFIMGIDGGVALSVLPMYLSEISPKEIRGSLGQVTAIFICIGV | |
| FTGQLLGLPELLGKESTWPYLFGVIVVPAVVQLLSLPFLPDSPRYLLLEKHNE | |
| ARAVKAFQTFLGKADVSQEVEEVLAESRVQRSIRLVSVLELLRAPYVRWQVV | |
| TVIVTMACYQLCGLNAIWFYTNSIFGKAGIPLAKIPYVTLSTGGIETLAAVFSG | |
| LVIEHLGRRPLLIGGFGLMGLFFGTLTITLTLQDHAPWVPYLSIVGILAIIASFC | |
| SGPGGIPFILTGEFFQQSQRPAAFIIAGTVNWLSNFAVGLLFPFIQKSLDTYCF | |
| LVFATICITGAIYLYFVLPETKNRTYAEISQAFSKRNKAYPPEEKIDSAVTDGKI | |
| NGRP | |
| B. Human GLUT9 (SLC2A9) Nucleic Acid (coding) Sequence | |
| (SEQ ID NO: 6) | |
| 1 | cttggcagag tctggggtcc ctggactgag ccatcagctg ggtcactgag acccatggca |
| 61 | aggaaacaaa ataggaattc caaggaactg ggcctagttc ccctcacaga tgacaccagc |
| 121 | cacgccaggc ctccagggcc agggagggca ctgctggagt gtgaccacct gaggagtggg |
| 181 | gtgccaggtg gaaggagaag aaaggactgg tcctgctcgc tcctcgtggc ctccctcgcg |
| 241 | ggcgccttcg gctcctcctt cctctacggc tacaacctgt cggtggtgaa tgcccccacc |
| 301 | ccgtacatca aggcctttta caatgagtca tgggaaagaa ggcatggacg tccaatagac |
| 361 | ccagacactc tgactctgct ctggtctgtg actgtgtcca tattcgccat cggtggactt |
| 421 | gtggggacat taattgtgaa gatgattgga aaggttcttg ggaggaagca cactttgctg |
| 481 | gccaataatg ggtttgcaat ttctgctgca ttgctgatgg cctgctcgct ccaggcagga |
| 541 | gcctttgaaa tgctcatcgt gggacgcttc atcatgggca tagatggagg cgtcgccctc |
| 601 | agtgtgctcc ccatgtacct cagtgagatc tcacccaagg agatccgtgg ctctctgggg |
| 661 | caggtgactg ccatctttat ctgcattggc gtgttcactg ggcagcttct gggcctgccc |
| 721 | gagctgctgg gaaaggagag tacctggcca tacctgtttg gagtgattgt ggtccctgcc |
| 781 | gttgtccagc tgctgagcct tccctttctc ccggacagcc cacgctacct gctcttggag |
| 841 | aagcacaacg aggcaagagc tgtgaaagcc ttccaaacgt tcttgggtaa agcagacgtt |
| 901 | tcccaagagg tagaggaggt cctggctgag agccgcgtgc agaggagcat ccgcctggtg |
| 961 | tccgtgctgg agctgctgag agctccctac gtccgctggc aggtggtcac cgtgattgtc |
| 1021 | accatggcct gctaccagct ctgtggcctc aatgcaattt ggttctatac caacagcatc |
| 1081 | tttggaaaag ctgggatccc tctggcaaag atcccatacg tcaccttgag tacagggggc |
| 1141 | atcgagactt tggctgccgt cttctctggt ttggtcattg agcacctggg acggagaccc |
| 1201 | ctcctcattg gtggctttgg gctcatgggc ctcttctttg ggaccctcac catcacgctg |
| 1261 | accctgcagg accacgcccc ctgggtcccc tacctgagta tcgtgggcat tctggccatc |
| 1321 | atcgcctctt tctgcagtgg gccaggtggc atcccgttca tcttgactgg tgagttcttc |
| 1381 | cagcaatctc agcggccggc tgccttcatc attgcaggca ccgtcaactg gctctccaac |
| 1441 | tttgctgttg ggctcctctt cccattcatt cagaaaagtc tggacaccta ctgtttccta |
| 1501 | gtctttgcta caatttgtat cacaggtgct atctacctgt attttgtgct gcctgagacc |
| 1561 | aaaaacagaa cctatgcaga aatcagccag gcattttcca aaaggaacaa agcataccca |
| 1621 | ccagaagaga aaatcgactc agctgtcact gatggtaaga taaatggaag gccttaacaa |
| 1681 | gtttcctcct ccacgttgga caattatgtc aaaaacagga ttgtctacat ggatgatctc |
| 1741 | acttttcagg aaacttaaaa tttacccatt attgggaagc ttaaatgaat tgaagctatg |
| 1801 | caagtctttt atattattaa atatttaaaa gtaaacctgt actaatctaa aaaaaaaaaa |
| 1861 | aaa |
| 4. (SWI1-like) (ARID1B) Gene | |
| A. Human (SWI1-like) (ARID1B) Polypeptide Sequence | |
| (SEQ ID NO: 7) | |
| MAHNAGAAAAAGTHSAKSGGSEAALKEGGSAAALSSSSSSSAAAAAASS | |
| SSSSGPGSAMETGLLPNHKLKTVGEAPAAPPHQQHHHHHHAHHHHHH | |
| AHHLHHHHALQQQLNQFQQQQQQQQQQQQQQQQQQHPISNNNSLGG | |
| AGGGAPQPGPDMEQPQHGGAKDSAAGGQADPPGPPLLSKPGDEDDAP | |
| PKMGEPAGGRYEHPGLGALGTQQPPVAVPGGGGGPAAVPEFNNYYGS | |
| AAPASGGPGGRAGPCFDQHGGQQSPGMGMMHSASAAAAGAPGSMDPL | |
| QNSHEGYPNSQCNHYPGYSRPGAGGGGGGGGGGGGGSGGGGGGGGA | |
| GAGGAGAGAVAAAAAAAAAAAGGGGGGGYGGSSAGYGVLSSPRQQGGG | |
| MMMGPGGGGAASLSKAAAGSAAGGFQRFAGQNQHPSGATPTLNQLLT | |
| SPSPMMRSYGGSYPEYSSPSAPPPPPSQPQSQAAAAGAAAGGQQAAAG | |
| MGLGKDMGAQYAAASPAWAAAQQRSHPAMSPGTPGPTMGRSQGSPM | |
| DPMVMKRPQLYGMGSNPHSQPQQSSPYPGGSYGPPGPQRYPIGIQGRT | |
| PGAMAGMQYPQQQDSGDATWKETFWLMPPQYGQQGVSGYCQQGQQP | |
| YYSQQPQPPHLPPQAQYLPSQSQQRYQPQQDMSQEGYGTRSQPPLAPG | |
| KPNHEDLNLIQQERPSSLPDLSGSIDDLPTGTEATLSSAVSASGSTSSQG | |
| DQSNPAQSPFSPHASPHLSSIPGGPSPSPVGSPVGSNQSRSGPISPASIPG | |
| SQMPPQPPGSQSESSSHPALSQSPMPQERGFMAGTQRNPQMAQYGPQ | |
| QTGPSMSPHPSPGGQMHAGISSFQQSNSSGTYGPQMSQYGPQGNYSRP | |
| PAYSGVPSASYSGPGPGMGISANNQMHGQGPSQPCGAVPLGRMPSAGM | |
| QNRPFPGNMSSMTPSSPGMSQQGGPGMGPPMPTVNRKAQEAAAAVM | |
| QAAANSAQSRQGSFPGMNQSGLMASSSPYSQPMNNSSSLMNTQAPPYS | |
| MAPAMVNSSAASVGLADMMSPGESKLPLPLKADGKEEGTPQPESKSKK | |
| SSSSTTTGEKITKVYELGNEPERKLWVDRYLTFMEERGSPVSSLPAVGK | |
| KPLDLFRLYVCVKEIGGLAQVNKNKKWRELATNLNVGTSSSAASSLKKQ | |
| YIQYLFAFECKIERGEEPPPEVFSTGDTKKQPKLQPPSPANSGSLQGPQ | |
| TPQSTGSNSMAEVPGDLKPPTPASTPHGQMTPMQGGRSSTISVHDPFS | |
| DVSDSSFPKRNSMTPNAPYQQGMSMPDVMGRMPYEPNKDPFGGMRK | |
| VPGSSEPFMTQGQMPNSSMQDMYNQSPSGAMSNLGMGQRQQFPYGAS | |
| YDRRHEPYGQQYPGQGPPSGQPPYGGHQPGLYPQQPNYKRHMDG | |
| MYGPPAKRHEGDMYNMQYSSQQQEMYNQYGGSYSGPDRRPIQGQYPY | |
| PYSRERMQGPGQIQTHGIPPQMMGGPLQSSSSEGPQQNMWAARNDMP | |
| YPYQNRQGPGGPTQAPPYPGMNRTDDMMVPDQRINHESQWPSHVSQR | |
| QPYMSSSASMQPITRPPQPSYQTPPSLPNHISRAPSPASFQRSLENRMSP | |
| SKSPFLPSMKMQKVMPTVPTSQVTGPPPQPPPIRREITFPPGSVEASQP | |
| VLKQRRKITSKDIVTPEAWRVMMSLKSGLLAESTWALDTINILLYDDSTV | |
| ATFNLSQLSGFLELLVEYFRKCLIDIFGILMEYEVGDPSQKALDHNAARK | |
| DDSQSLADDSGKEEEDAECIDDDEEDEEDEEEDSEKTESDEKSSIALTA | |
| PDAAADPKEKPKQASKFDKLPIKIVKKNNLFVVDRSDKLGRVQEFNSGL | |
| LHWQLGGGDTTEHIQTHFESKMEIPPRRPPPPLSSAGRKKEQEGKGDS | |
| EEQQEKSIIATIDDVLSARPGALPEDANPGPQTESSKFPFGIQQAKSHRN | |
| IKLLEDEPRSRDETPLCTIAHWQDSLAKRCICVSNIVRSLSFVPGNDAEM | |
| SKHPGLVLILGKLILLHHEHPERKRAPQTYEKEEDEDKGVACSKDEWW | |
| WDCLEVLRDNTLVTLANISGQLDLSAYTESICLPILDGLLHWMVCPSAE | |
| AQDPFPTVGPNSVLSPQRLVLETLCKLSIQDNNVDLILATPPFSRQEKFY | |
| ATLVRYVGDRKNPVCREMSMALLSNLAQGDALAARAIAVQKGSIGNLIS | |
| FLEDGVTMAQYQQSQHNLMHMQPPPLEPPSVDMMCRAAKALLAMARV | |
| DENRSEFLLHEGRLLDISISAVLNSLVASVICDVLFQIGQL | |
| B. Human (SWI1-like) (ARID1B) Nucleic Acid Sequence | |
| (SEQ ID NO: 8) | |
| 1 | atggcccata acgcgggcgc cgcggccgcc gccggcaccc acagcgccaa gagcggcggc |
| 61 | tccgaggcgg ctctcaagga gggtggaagc gccgccgcgc tgtcctcctc ctcctcctcc |
| 121 | tccgcggcgg cagcggcggc atcctcttcc tcctcgtcgg gcccgggctc ggccatggag |
| 181 | acggggctgc tccccaacca caaactgaaa accgttggcg aagcccccgc cgcgccgccc |
| 241 | caccagcagc accaccacca ccaccatgcc caccaccacc accaccatgc ccaccacctc |
| 301 | caccaccacc acgcactaca gcagcagcta aaccagttcc agcagcagca gcagcagcag |
| 361 | caacagcagc agcagcagca gcagcaacag caacatccca tttccaacaa caacagcttg |
| 421 | ggcggcgcgg gcggcggcgc gcctcagccc ggccccgaca tggagcagcc gcaacatgga |
| 481 | ggcgccaagg acagtgctgc gggcggccag gccgaccccc cgggcccgcc gctgctgagc |
| 541 | aagccgggcg acgaggacga cgcgccgccc aagatggggg agccggcggg cggccgctac |
| 601 | gagcacccgg gcttgggcgc cctgggcacg cagcagccgc cggtcgccgt gcccgggggc |
| 661 | ggcggcggcc cggcggccgt cccggagttt aataattact atggcagcgc tgcccctgcg |
| 721 | agcggcggcc ccggcggccg cgctgggcct tgctttgatc aacatggcgg acaacaaagc |
| 781 | cccgggatgg ggatgatgca ctccgcctcc gccgccgccg ccggggcccc cggcagcatg |
| 841 | gaccccctgc agaactccca cgaagggtac cccaacagcc agtgcaacca ttatccgggc |
| 901 | tacagccggc ccggcgcggg cggcggcggc ggcggcggcg gcggaggagg aggaggcagc |
| 961 | ggaggaggag gaggaggagg aggagcagga gcaggaggag caggagcggg agctgtggcg |
| 1021 | gcggcggccg cggcggcggc ggcagcagca ggaggcggcg gcggcggcgg ctatgggggc |
| 1081 | tcgtccgcgg ggtacggggt gctgagctcc ccccggcagc agggcggcgg catgatgatg |
| 1141 | ggccccgggg gcggcggggc cgcgagcctc agcaaggcgg ccgccggctc ggcggcgggg |
| 1201 | ggcttccagc gcttcgccgg ccagaaccag cacccgtcgg gggccacccc gaccctcaat |
| 1261 | cagctgctca cctcgcccag ccccatgatg cggagctacg gcggcagcta ccccgagtac |
| 1321 | agcagcccca gcgcgccgcc gccgccgccg tcgcagcccc agtcccaggc ggcggcggcg |
| 1381 | ggggcggcgg cgggcggcca gcaggcggcc gcgggcatgg gcttgggcaa ggacatgggc |
| 1441 | gcccagtacg ccgctgccag cccggcctgg gcggccgcgc aacaaaggag tcacccggcg |
| 1501 | atgagccccg gcacccccgg accgaccatg ggcagatccc agggcagccc aatggatcca |
| 1561 | atggtgatga agagacctca gttgtatggc atgggcagta accctcattc tcagcctcag |
| 1621 | cagagcagtc cgtacccagg aggttcctat ggccctccag gcccacagcg gtatccaatt |
| 1681 | ggcatccagg gtcggactcc cggggccatg gccggaatgc agtaccctca gcagcaggac |
| 1741 | tctggagatg ccacatggaa agaaacattc tggttgatgc cacctcagta tggacagcaa |
| 1801 | ggtgtgagtg gttactgcca gcagggccaa cagccatatt acagccagca gccgcagccc |
| 1861 | ccgcacctcc caccccaggc gcagtatctg ccgtcccagt cccagcagag gtaccagccg |
| 1921 | cagcaggaca tgtctcagga aggctatgga actagatctc aacctcctct ggcccccgga |
| 1981 | aaacctaacc atgaagactt gaacttaata cagcaagaaa gaccatcaag tttaccagat |
| 2041 | ctgtctggct ccattgatga cctccccacg ggaacggaag caactttgag ctcagcagtc |
| 2101 | agtgcatccg ggtccacgag cagccaaggg gatcagagca acccggcgca gtcgcctttc |
| 2161 | tccccacatg cgtcccctca tctctccagc atcccggggg gcccatctcc ctctcctgtt |
| 2221 | ggctctcctg taggaagcaa ccagtctcga tctggcccaa tctctcctgc aagtatccca |
| 2281 | ggtagtcaga tgcctccgca gccacccggg agccagtcag aatccagttc ccatcccgcc |
| 2341 | ttgagccagt caccaatgcc acaggaaaga ggttttatgg caggcacaca aagaaaccct |
| 2401 | cagatggctc agtatggacc tcaacagaca ggaccatcca tgtcgcctca tccttctcct |
| 2461 | gggggccaga tgcatgctgg aatcagtagc tttcagcaga gtaactcaag tgggacttac |
| 2521 | ggtccacaga tgagccagta tggaccacaa ggtaactact ccagaccccc agcgtatagt |
| 2581 | ggggtgccca gtgcaagcta cagcggccca gggcccggta tgggtatcag tgccaacaac |
| 2641 | cagatgcatg gacaagggcc aagccagcca tgtggtgctg tgcccctggg acgaatgcca |
| 2701 | tcagctggga tgcagaacag accatttcct ggaaatatga gcagcatgac ccccagttct |
| 2761 | cctggcatgt ctcagcaggg agggccagga atggggccgc caatgccaac tgtgaaccgt |
| 2821 | aaggcacagg aggcagccgc agcagtgatg caggctgctg cgaactcagc acaaagcagg |
| 2881 | caaggcagtt tccccggcat gaaccagagt ggacttatgg cttccagctc tccctacagc |
| 2941 | cagcccatga acaacagctc tagcctgatg aacacgcagg cgccgcccta cagcatggcg |
| 3001 | cccgccatgg tgaacagctc ggcagcatct gtgggtcttg cagatatgat gtctcctggt |
| 3061 | gaatccaaac tgcccctgcc tctcaaagca gacggcaaag aagaaggcac tccacagccc |
| 3121 | gagagcaagt caaagaagtc cagctcctcc accactactg gggagaagat cacgaaggtg |
| 3181 | tacgagctgg ggaatgagcc agagagaaag ctctgggtcg accgatacct caccttcatg |
| 3241 | gaagagagag gctctcctgt ctcaagtctg cctgccgtgg gcaagaagcc cctggacctg |
| 3301 | ttccgactct acgtctgcgt caaagagatc gggggtttgg cccaggttaa taaaaacaag |
| 3361 | aagtggcgtg agctggcaac caacctaaac gttggcacct caagcagtgc agcgagctcc |
| 3421 | ctgaaaaagc agtatattca gtacctgttt gcctttgagt gcaagatcga acgtggggag |
| 3481 | gagcccccgc cggaagtctt cagcaccggg gacaccaaaa agcagcccaa gctccagccg |
| 3541 | ccatctcctg ctaactcggg atccttgcaa ggcccacaga ccccccagtc aactggcagc |
| 3601 | aattccatgg cagaggttcc aggtgacctg aagccaccta ccccagcctc cacccctcac |
| 3661 | ggccagatga ctccaatgca aggtggaaga agcagtacaa tcagtgtgca cgacccattc |
| 3721 | tcagatgtga gtgattcatc cttcccgaaa cggaactcca tgactccaaa cgccccctac |
| 3781 | cagcagggca tgagcatgcc cgatgtgatg ggcaggatgc cctatgagcc caacaaggac |
| 3841 | ccctttgggg gaatgagaaa agtgcctgga agcagcgagc cctttatgac gcaaggacag |
| 3901 | atgcccaaca gcagcatgca ggacatgtac aaccaaagtc cctccggagc aatgtctaac |
| 3961 | ctgggcatgg ggcagcgcca gcagtttccc tatggagcca gttacgaccg aaggcatgaa |
| 4021 | ccttatgggc agcagtatcc aggccaaggc cctccctcgg gacagccgcc gtatggaggg |
| 4081 | caccagcccg gcctgtaccc acagcagccg aattacaaac gccatatgga cggcatgtac |
| 4141 | gggcccccag ccaagcgcca cgagggcgac atgtacaaca tgcagtacag cagccagcag |
| 4201 | caggagatgt acaaccagta tggaggctcc tactcgggcc cggaccgcag gcccatccag |
| 4261 | ggccagtacc cgtatcccta cagcagggag aggatgcagg gcccggggca gatccagaca |
| 4321 | cacggaatcc cgcctcagat gatgggcggc ccgctgcagt cgtcctccag tgaggggcct |
| 4381 | cagcagaata tgtgggcagc acgcaatgat atgccttatc cctaccagaa caggcagggc |
| 4441 | cctggcggcc ctacacaggc gcccccttac ccaggcatga accgcacaga cgatatgatg |
| 4501 | gtacccgatc agaggataaa tcatgagagc cagtggcctt ctcacgtcag ccagcgtcag |
| 4561 | ccttatatgt cgtcctcagc ctccatgcag cccatcacac gcccaccaca gccgtcctac |
| 4621 | cagacgccac cgtcactgcc aaatcacatc tccagggcgc ccagcccagc gtccttccag |
| 4681 | cgctccctgg agaaccgcat gtctccaagc aagtctcctt ttctgccgtc tatgaagatg |
| 4741 | cagaaggtca tgcccacggt ccccacatcc caggtcaccg ggccaccacc ccaaccaccc |
| 4801 | ccaatcagaa gggagatcac ctttcctcct ggctcagtag aagcatcaca accagtcttg |
| 4861 | aaacaaaggc gaaagattac ctccaaagat atcgttactc ctgaggcgtg gcgtgtgatg |
| 4921 | atgtccctta aatcaggtct tttggctgag agtacgtggg ctttggacac tattaatatt |
| 4981 | cttctgtatg atgacagcac tgttgctact ttcaatctct cccagttgtc tggatttctc |
| 5041 | gaacttttag tcgagtactt tagaaaatgc ctgattgaca tttttggaat tcttatggaa |
| 5101 | tatgaagtgg gagaccccag ccaaaaagca cttgatcaca acgcagcaag gaaggatgac |
| 5161 | agccagtcct tggcagacga ttctgggaaa gaggaggaag atgctgaatg tattgatgac |
| 5221 | gacgaggaag acgaggagga tgaggaggaa gacagcgaga agacagaaag cgatgaaaag |
| 5281 | agcagcatcg ctctgactgc cccggacgcc gctgcagacc caaaggagaa gcccaagcaa |
| 5341 | gccagtaagt tcgacaagct gccaataaag atagtcaaaa agaacaacct gtttgttgtt |
| 5401 | gaccgatctg acaagttggg gcgtgtgcag gagttcaata gtggccttct gcactggcag |
| 5461 | ctcggcgggg gtgacaccac cgagcacatt cagactcact ttgagagcaa gatggaaatt |
| 5521 | cctcctcgca ggcgcccacc tcccccctta agctccgcag gtagaaagaa agagcaagaa |
| 5581 | ggcaaaggcg actctgaaga gcagcaagag aaaagcatca tagcaaccat cgatgacgtc |
| 5641 | ctctctgctc ggccaggggc attgcctgaa gacgcaaacc ctgggcccca gaccgaaagc |
| 5701 | agtaagtttc cctttggtat ccagcaagcc aaaagtcacc ggaacatcaa gctgctggag |
| 5761 | gacgagccca ggagccgaga cgagactcct ctgtgtacca tcgcgcactg gcaggactcg |
| 5821 | ctggctaagc gatgcatctg tgtgtccaat attgtccgta gcttgtcatt cgtgcctggc |
| 5881 | aatgatgccg aaatgtccaa acatccaggc ctggtgctga tcctggggaa gctgattctt |
| 5941 | cttcaccacg agcatccaga gagaaagcga gcaccgcaga cctatgagaa agaggaggat |
| 6001 | gaggacaagg gggtggcctg cagcaaagat gagtggtggt gggactgcct cgaggtcttg |
| 6061 | agggataaca cgttggtcac gttggccaac atttccgggc agctagactt gtctgcttac |
| 6121 | acggaaagca tctgcttgcc aattttggat ggcttgctgc actggatggt gtgcccgtct |
| 6181 | gcagaggcac aagatccctt tccaactgtg ggacccaact cggtcctgtc gcctcagaga |
| 6241 | cttgtgctgg agaccctctg taaactcagt atccaggaca ataatgtgga cctgatcttg |
| 6301 | gccactcctc catttagtcg tcaggagaaa ttctatgcta cattagttag gtacgttggg |
| 6361 | gatcgcaaaa acccagtctg tcgagaaatg tccatggcgc ttttatcgaa ccttgcccaa |
| 6421 | ggggacgcac tagcagcaag ggccatagct gtgcagaaag gaagcattgg aaacttgata |
| 6481 | agcttcctag aggatggggt cacgatggcc cagtaccagc agagccagca caacctcatg |
| 6541 | cacatgcagc ccccgcccct ggaaccacct agcgtagaca tgatgtgcag ggcggccaag |
| 6601 | gctttgctag ccatggccag agtggacgaa aaccgctcgg aattcctttt gcacgagggc |
| 6661 | cggttgctgg atatctcgat atcagctgtc ctgaactctc tggttgcatc tgtcatctgt |
| 6721 | gatgtactgt ttcagattgg gcagttatga cataagtgag aaggcaagca tgtgtgagtg |
| 6781 | aagattagag ggtcacatat aactggctgt tttctgttct tgtttatcca gcgtaggaag |
| 6841 | aaggaaaaga aaatctttgc tcctctgccc cattcactat ttaccaattg ggaattaaag |
| 6901 | aaataattaa tttgaacagt tatgaaatta atatttgctg tctgtgtgta taagtacatc |
| 6961 | ctttggggtt ttttttttct ctttttttta accaaagttg ctgtctagtg cattcaaagg |
| 7021 | tcactttttg ttcttcacag atctttttaa tgttctttcc catgttgtat tgcatttttg |
| 7081 | ggggaagcaa attgacttta aagaaaaaag ttgtggcaaa agatgctaag atgcgaaaat |
| 7141 | ttcaccacac tgagtcaaaa aggtgaaaaa ttatccattt cctatgcgtt ttactcctca |
| 7201 | gagaatgaaa aaaactgcat cccatcaccc aaagttctgt gcaatagaaa tttctacaga |
| 7261 | tacaggtata ggggctcaag gaggtatgtc ggtcagtagt caaaactatg aaatgatact |
| 7321 | ggtttctcca caggaatatg gttccattag gctgggagca aaaacaatgt tttttaagat |
| 7381 | tgagaataca tacctgacaa cgatccggaa actgctcctc accactcccg tcatgcctgc |
| 7441 | tgtcggcgtt tgaccttcca cgtgacagtt cttcacaatt cctttcatca ttttttaaat |
| 7501 | atttttttta ctgcctatgg gctgtgatgt atatagaagt tgtacattaa acataccctc |
| 7561 | atttttttct tttctttttt tttttttttt ttagtacaaa gttttagttt ctttttcatg |
| 7621 | atgtggtaac tacgaagtga tggtagattt aaataatttt ttatttttat tttatatatt |
| 7681 | ttttcattag ggccatatct ccaaaaaaag aaagaaaaaa tacaaaaaac aaaaacaaaa |
| 7741 | aaaaaagagg gtaatgtaca agtttctgta tgtataaagt catgctcgat ttcaggagag |
| 7801 | cagctgatca caatttgctt catgaatcaa ggtgtggaaa tggttatata tggattgatt |
| 7861 | tagaaaatgg ttaccagtac agtcaaaaaa gagaaaatga aaaaaataca actaaaagga |
| 7921 | agaaacacaa cttcaaagat ttttcagtga tgagaatcca catttgtatt tcaagataat |
| 7981 | gtagtttaaa aaaaaaaaaa agaaaaaaac ttgatgtaaa ttcctccttt tcctctggct |
| 8041 | taatgaatat catttattca gtataaaatc tttatatgtt ccacatgtta agaataaatg |
| 8101 | tacattaaat cttgttaagc actgtgatgg gtgttcttga atactgttct agtttcctta |
| 8161 | aagtggtttc ctagtaatca agttatttac aagaaatagg ggaatgcagc agtgtattca |
| 8221 | cattataaaa ccctacattt ggaagagacc tttaggggtt acctacttta gagtggggag |
| 8281 | caacagtttg attttctcaa attacttagc taattagtct ttctttgaag caattaactc |
| 8341 | taacgacatt gaggtatgat cattttcagt atttatggga ggtggctgct gacccacttg |
| 8401 | aggtgagatc tcagaagctt aactggcctg aaaatgtaac attctgcctt ttactaactc |
| 8461 | catcttagtt taatcaaagt tcaatctatt ccttgtttct tctgtgtgcc tcagagttat |
| 8521 | tttgcattta gtttactcca ccgtgtataa tatttatact gtgcaatgtt aaaaaagaat |
| 8581 | ctgttatatt gtatgtggtg tacatagtgc aaagtgatga tttctatttc agggcatatt |
| 8641 | atggttctca tattccttcc tacctggtgc acagtagctt tttaatacta gtcacttcta |
| 8701 | atttaaactt tctcttcctg ggtcattgac tgttactgtg taataatcga tttctttgaa |
| 8761 | actgctgcat aattatgctg ttagtggacc tctacctctt ctcttccctc tcccaatcac |
| 8821 | agtatactca gaatccccag cccctcgcat acattgtgtc ggttcacatt actcacagta |
| 8881 | atatatggaa gagttagaca agaacatgca gttacagtca ttgtgagacg tgactctcca |
| 8941 | gtgtcacgag gaaaaaaatc atcttttctg caaacagtct ctcatctgtc aactcccaca |
| 9001 | ttactgagtc aaacagtctt cttacataac aatgcaacca aatatatgtt gaattaaaga |
| 9061 | cccatttata attctgcttt aaatacatct gcttgctaag aacagatttc agtgctccaa |
| 9121 | gcttcaaata tggagatttg taagagggaa ttcaatatta ttctaatttc tctcttacag |
| 9181 | agtacaaata aaaggtgtat acaaactccg aacatatcca gtattccaat tcctttgtca |
| 9241 | atcagaagag taaaataatt aacaaaagac tgttgttatg gtttgcattg taaccgatac |
| 9301 | gcagagtctg accgttgggc aacaagtttt tctatcctga tgcgcaacac agtctctaga |
| 9361 | gactaatcca ggaagacttt agcctccttt ccatattctc acccccgaat caagatttac |
| 9421 | agaagcccac gaagaattta cagcctgctt gagatcatct tgcctataaa ctgagttatt |
| 9481 | gctttgtcct aaaaattagt cggttttttt ttttctatga ggcttttcag aaatttacag |
| 9541 | gatgcccaga ctttacatgt gtaccaaaaa aaaaaaaaag ataaaaaata aaggtgcaaa |
| 9601 | gaaagtttag tattttggaa tggtgctata aagttgaaaa aaaaaaaa |
| 5. FAM36A Gene | |
| A. Human FAM36A Polypeptide Sequence | |
| (SEQ ID NO: 9) | |
| MAAPPEPGEPEERKSLKLLGFLDVENTPCARHSILYGSLGSVVA | |
| GFGHFLFTSRIRRSCDVGVGGFILVTLGCWFHCRYNYAKQRIQERIAREEIKK | |
| KILYE GTHLDPERKHNGSSSN | |
| B. Human FAM36A Nucleic Acid (mRNA) Sequence | |
| (SEQ ID NO: 10) | |
| 1 | ggtggagtcg cggagtagtc ctcatggccg ccccgccgga gcccggtgag cccgaggaga |
| 61 | ggaagtccct taagctccta ggatttttag atgttgaaaa tactccctgc gcccggcatt |
| 121 | caatattgta tggttcatta ggatctgttg tggctggctt tggacatttt ttgttcacta |
| 181 | gtagaattag aagatcatgt gatgttggag taggagggtt tatcttggtg actttgggat |
| 241 | gctggtttca ttgtaggtat aattatgcaa agcaaagaat ccaggaaaga attgccagag |
| 301 | aagaaattaa aaagaagata ttatatgaag gtacccacct cgatcctgaa agaaaacaca |
| 361 | acggcagcag cagcaattga acaatcttga gcatagaagt caatgtaaac gaagttaaga |
| 421 | tcaaccacat aaaacatttc atgtgcaata agctctcaat caagtaaata aagtttaagt |
| 481 | tgtagtcatt tttttcccac acttgtgtgg aatgaaaact tgccagttta ttctggccct |
| 541 | gtgtctactg ccaggatagc attcttacgt gttacatata gtggacttgt catccttaaa |
| 601 | atgtgaacag aatttattgg cagtgtggca aagaattata aaacatagtg tttaatgtac |
| 661 | ttggagtttc cttgtagtag taagtataga gtttgatgat aagtaaacgt cccttaacaa |
| 721 | aaacctcaac cttattacta tcccattaaa aaacagcaaa tacttactga gttcttgtaa |
| 781 | gagctaatgt cattgtaaga tttaaaacta agggctttta tcactttgca aattattttt |
| 841 | taaatgcatt catcatttga cagtgttctc tcatttctta aaatgcgagt catcttccaa |
| 901 | aagagttgtt tttaactgcc ctaaacattt ttggggaagt atgcagggtt taaattttta |
| 961 | agtataatta gttctgaatt aaaatatgca aaaaaaaaaa aaaaaaaaaa aaaaaaaaa |
| 6. NR2F6 Gene | |
| A. Human NR2F6 Polypeptide Sequence | |
| (SEQ ID NO: 11) | |
| MAMVTGGWGGPGGDTNGVDKAGGYPRAAEDDSASPPGAASDAEPGDEERP | |
| GLQVDCVVCGDKSSGKHYGVFTCEGCKSFFKRSIRRNLSYTCRSNRDCQIDQ | |
| HHRNQCQYCRLKKCFRVGMRKEAVQRGRIPHSLPGAVAASSGSPPGSALAAV | |
| ASGGDLFPGQPVSELIAQLLRAEPYPAAAGRFGAGGGAAGAVLGIDNVCELA | |
| ARLLFSTVEWARHAPFFPELPVADQVALLRLSWSELFVLNAAQAALPLHTAP | |
| LLAAAGLHAAPMAAERAVAFMDQVRAFQEQVDKLGRLQVDSAEYGCLKAIA | |
| LFTPDACGLSDPAHVESLQEKAQVALTEYVRAQYPSQPQRFGRLLLRLPALR | |
| AVPASLISQLFFMRLVGKTPIETLIRDMLLSGSTFNWPYGSGQ | |
| B. Human NR2F6 Nucleic acid (mRNA) Sequence | |
| (SEQ ID NO: 12) | |
| 1 | gtgcagcccg tgccccccgc gcgccggggc cgaatgcgcg ccgcgtaggg tcccccgggc |
| 61 | cgagaggggt gcccggaggg aagagcgcgg tgggggcgcc ccggccccgc tgccctgggg |
| 121 | ctatggccat ggtgaccggc ggctggggcg gccccggcgg cgacacgaac ggcgtggaca |
| 181 | aggcgggcgg ctacccgcgc gcggccgagg acgactcggc ctcgcccccc ggtgccgcca |
| 241 | gcgacgccga gccgggcgac gaggagcggc cggggctgca ggtggactgc gtggtgtgcg |
| 301 | gggacaagtc gagcggcaag cattacggtg tcttcacctg cgagggctgc aagagctttt |
| 361 | tcaagcgaag catccgccgc aacctcagct acacctgccg gtccaaccgt gactgccaga |
| 421 | tcgaccagca ccaccggaac cagtgccagt actgccgtct caagaagtgc ttccgggtgg |
| 481 | gcatgaggaa ggaggcggtg cagcgcggcc gcatcccgca ctcgctgcct ggtgccgtgg |
| 541 | ccgcctcctc gggcagcccc ccgggctcgg cgctggcggc agtggcgagc ggcggagacc |
| 601 | tcttcccggg gcagccggtg tccgaactga tcgcgcagct gctgcgcgct gagccctacc |
| 661 | ctgcggcggc cggacgcttc ggcgcagggg gcggcgcggc gggcgcggtg ctgggcatcg |
| 721 | acaacgtgtg cgagctggcg gcgcggctgc tcttcagcac cgtggagtgg gcgcgccacg |
| 781 | cgcccttctt ccccgagctg ccggtggccg accaggtggc gctgctgcgc ctgagctgga |
| 841 | gcgagctctt cgtgctgaac gcggcgcagg cggcgctgcc cctgcacacg gcgccgctac |
| 901 | tggccgccgc cggcctccac gccgcgccta tggccgccga gcgcgccgtg gctttcatgg |
| 961 | accaggtgcg cgccttccag gagcaggtgg acaagctggg ccgcctgcag gtcgactcgg |
| 1021 | ccgagtatgg ctgcctcaag gccatcgcgc tcttcacgcc cgacgcctgt ggcctctcag |
| 1081 | acccggccca cgttgagagc ctgcaggaga aggcgcaggt ggccctcacc gagtatgtgc |
| 1141 | gggcgcagta cccgtcccag ccccagcgct tcgggcgcct gctgctgcgg ctccccgccc |
| 1201 | tgcgcgcggt ccctgcctcc ctcatctccc agctgttctt catgcgcctg gtggggaaga |
| 1261 | cgcccattga gacactgatc agagacatgc tgctgtcggg gagtaccttc aactggccct |
| 1321 | acggctcggg ccagtgacca tgacggggcc acgtgtgctg tggccaggcc tgcagacaga |
| 1381 | cctcaaggga cagggaatgc tgaggcctcg aggggcctcc cggggcccag gactctggct |
| 1441 | tctctcctca gacttctatt ttttaaagac tgtgaaatgt ttgtcttttc tgttttttaa |
| 1501 | atgatcatga aaccaaaaag agactgatca tccaggcctc agcctcatcc tccccaggac |
| 1561 | ccctgtccag gatggagggt ccaatcctag gacagccttg ttcctcagca cccctagcat |
| 1621 | gaacttgtgg gatggtgggg ttggcttccc tggcatgatg gacaaaggcc tggcgtcggc |
| 1681 | cagaggggct gctccagtgg gcaggggtag ctagcgtgtg ccaggcagat cctctggaca |
| 1741 | cgtaacctat gtcagacact acatgatgac tcaaggccaa taataaagac atttcctacc |
| 1801 | tgca |
| 7. ZNF132 Gene | |
| A. Human ZNF132 Polypeptide Sequence | |
| (SEQ ID NO: 13) | |
| MCGPFLKDILHLAEHQGTQSEEKPYTCGACGRDFWLNANLHQHQKEHSGG | |
| KPFRWYKDRDALMKSSKVHLSENPFTCREGGKVILGSCDLLQLQAVDSGQK | |
| PYSNLGQLPEVCTTQKLFECSNCGKAFLKSSTLPNHLRTHSEEIPFTCPTGGN | |
| FLEEKSILGNKKFHTGEIPHVCKECGKAFSHSSKLRKHQKFHTEVKYYECIA | |
| CGKTFNHKLTFVHHQRIHSGERPYECDECGKAFSNRSHLIRHEKVHTGERPF | |
| ECLKCGRAFSQSSNFLRHQKVHTQVRPYECSQCGKSFSRSSALIQHWRVHTG | |
| ERPYECSECGRAFNNNSNLAQHQKVHTGERPFECSECGRDFSQSSHLLRHQ | |
| KVHTGERPFECCDCGKAFSNSSTLIQHQKVHTGQRPYECSECRKSFSRSSSLI | |
| QHWRIHTGEKPYECSECGKAFAHSSTLIEHWRVHTKERPYECNECGKFFSQ | |
| NSILIKHQKVHTGEKPYKCSECGKFFSRKSSLICHWRVHTGERPYECSECGR | |
| AFSSNSHLVRHQRVHTQERPYECIQCGKAFSERSTLVRHQKVHTRERTYECS | |
| QCGKLFSHLCNLAQHKKIHT | |
| B. Human ZNF132 Nucleic Acid (mRNA coding) Sequence | |
| (SEQ ID NO: 14) | |
| 1 | ctaaagctag tggatgtgaa gtggtatctc attatggttt tggttttcat actcctcatg |
| 61 | tttaaggatg ctgaacttct tttcatatgc ttattggcca tttgtgtata tatcttcttt |
| 121 | tagagaaatg tctatttaag tcctttgacc catttctgtg tccttacccc tggtgaggtc |
| 181 | tcccttattc tgttgcttgg ctggtcccta tcctgccaat agtaatgggc ccttcttcac |
| 241 | cctgatgatg gccctgttgg cctgtcagca atccctggga cctcttcttg ggtgtgaatt |
| 301 | cctgggtaac atttctaatg aagtcaacca ttcccaccaa gtggaattct tagttaactg |
| 361 | gcatttctct actttcaggt tcttggcaat ggagtagagg gtgagggggc ccatcccaag |
| 421 | cagaatgttt ctgtagaagt gttacaggtc aggatcccta atgcagatcc ttccaccaag |
| 481 | aaagctaact cctgtgacat gtgtgggcca ttcttgaaag acattttgca cctggctgag |
| 541 | catcagggaa cacagtctga ggagaaaccc tacacatgtg gagcatgtgg gagagacttt |
| 601 | tggttgaatg caaaccttca ccagcaccag aaggagcaca gtggagggaa gccctttaga |
| 661 | tggtacaagg acagggacgc acttatgaag agctctaaag tccacctgtc agagaacccc |
| 721 | ttcacttgca gggaaggtgg gaaggtcatc ctgggcagct gtgacctcct ccagcttcaa |
| 781 | gctgttgaca gtgggcagaa gccatattcc aatcttgggc agcttccaga agtctgtacc |
| 841 | acacagaaac tcttcgagtg cagcaactgt ggaaaagcct tcctgaagag ctccactctc |
| 901 | cccaaccatc tgagaactca ctctgaagag ataccattta catgcccaac aggtggaaat |
| 961 | ttcttagagg agaaatcaat ccttggtaat aaaaagtttc acactgggga aataccccat |
| 1021 | gtgtgtaagg agtgtgggaa ggcctttagt cactcatcta agctgaggaa gcaccagaaa |
| 1081 | tttcacactg aagtaaaata ttatgagtgc attgcatgtg ggaaaacctt caaccacaaa |
| 1141 | ctcacatttg ttcatcatca gagaattcac tcaggtgaaa gaccttatga gtgtgatgaa |
| 1201 | tgtgggaaag ccttcagtaa cagatcacac ctcattcggc atgagaaagt tcacactgga |
| 1261 | gaaaggcctt ttgagtgcct gaaatgtgga agagccttca gccaaagctc caatttcctt |
| 1321 | cggcatcaga aagttcacac acaggtaaga ccttatgagt gcagtcaatg tggtaaatcc |
| 1381 | ttcagccgaa gctctgctct cattcagcac tggagagttc acactggaga aagaccgtat |
| 1441 | gaatgcagtg aatgtggaag agcttttaac aataactcca accttgctca gcaccagaaa |
| 1501 | gttcacaccg gagaacggcc ttttgagtgc agtgaatgtg gaagagactt cagccaaagc |
| 1561 | tcccatctcc ttcgacatca gaaagttcac actggagaac ggccttttga atgctgtgat |
| 1621 | tgtggtaaag ccttcagtaa tagctccacc ctcatccagc accagaaagt acatactggg |
| 1681 | caaaggcctt atgagtgcag cgaatgtagg aaatccttca gccgcagctc cagcctgatt |
| 1741 | cagcactgga gaattcacac tggagaaaag ccttacgagt gtagtgagtg tgggaaagcc |
| 1801 | tttgctcaca gctccactct cattgaacac tggagagttc acacaaaaga aaggccttat |
| 1861 | gagtgcaatg aatgtgggaa attctttagc caaaactcca ttctcattaa gcatcagaaa |
| 1921 | gttcatactg gagaaaagcc ttataaatgc agtgaatgtg ggaaattctt tagccgaaaa |
| 1981 | tccagcctta tttgtcactg gagagttcac actggagaaa ggccttacga atgcagtgaa |
| 2041 | tgtgggagag cctttagcag taactcccac ctggttcgtc atcagagagt tcacacacaa |
| 2101 | gaaaggccct atgagtgcat ccagtgtgga aaagccttta gtgaaagatc tacacttgtt |
| 2161 | cggcaccaga aagttcacac cagagaaagg acttatgagt gtagccagtg tgggaaactc |
| 2221 | ttcagccatc tttgtaacct tgcacagcat aaaaagattc atacctgagt ggagccttat |
| 2281 | ggaagtggtc tttgtgagaa aatcttcagc caagtcaaac ttcatgcagc agaatcccca |
| 2341 | taccagaaaa attacctcca tgctttag |
| 8. MTUS1 Gene | |
| A. Human MTUS1 Polypeptide Sequence | |
| (SEQ ID NO: 15) | |
| MTDDNSDDKIEDELQTFFTSDKDGNTHAYNPKSPPTQNSSASSVNWNSANP | |
| DDMVVDYETDPAVVTGENISLSLQGVEVFGHEKSSSDFISKQVLDMHKDSIC | |
| QCPALVGTEKPKYLQHSCHSLEAVEGQSVEPSLPFVWKPNDNLNCAGYCDA | |
| LELNQTFDMTVDKVNCTFISHHAIGKSQSFHTAGSLPPTGRRSGSTSSLSYST | |
| WTSSHSDKTHARETTYDRESFENPQVTPSEAQDMTYTAFSDVVMQSEVFVS | |
| DIGNQCACSSGKVTSEYTDGSQQRLVGEKETQALTPVSDGMEVPNDSALQEF | |
| FCLSHDESNSEPHSQSSYRHKEMGQNLRETVSYCLIDDECPLMVPAFDKSEA | |
| QVLNPEHKVTETEDTQMVSKGKDLGTQNHTSELILSSPPGQKVGSSFGLTW | |
| DANDMVISTDKTMCMSTPVLEPTKVTFSVSPIEATEKCKKVEKGNRGLKNIP | |
| DSKEAPVNLCKPSLGKSTIKTNTPIGCKVRKTEIISYPRPNFKNVKAKVMSRA | |
| VLQPKDAALSKVTPRPQQTSASSPSSVNSRQQTVLSRTPRSDLNADKKAEILI | |
| NKTHKQQFNKLITSQAVHVTTHSKNASHRVPRTTSAVKSNQEDVDKASSSNS | |
| ACETGSVSALFQKIKGILPVKMESAECLEMTYVPNIDRISPEKKGEKENGTSM | |
| EKQELKQEIMNETFEYGSLFLGSASKTTTTSGRNISKPDSCGLRQIAAPKAKV | |
| GPPVSCLRRNSDNRNPSADRAVSPQRIRRVSSSGKPTSLKTAQSSWVNLPRPL | |
| PKSKASLKSPALRRTGSTPSIASTHSELSTYSNNSGNAAVIKYEEKPPKPAFQN | |
| GSSGSFYLKPLVSRAHVHLMKTPPKGPSRKNLFTALNAVEKSRQKNPRSLCI | |
| QPQTAPDALPPEKTLELTQYKTKCENQSGFILQLKQLLACGNTKFEALTVVIQ | |
| HLLSEREEALKQHKTLSQELVNLRGELVTASTTCEKLEKARNELQTVYEAFV | |
| QQHQAEKTERENRLKEFYTREYEKLRDTYIEEAEKYKMQLQEQFDNLNAAH | |
| ETSKLEIEASHSEKLELLKKAYEASLSEIKKGHEIEKKSLEDLLSEKQESLEK | |
| QINDLKSENDALNEKLKSEEQKRRAREKANLKNPQIMYLEQELESLKAVLEI | |
| KNEKLHQQDIKLMKMEKLVDNNTALVDKLKRFQQENEELKARMDKHMAIS | |
| RQLSTEQAVLQESLEKESKVNKRLSMENEELLWKLHNGDLCSPKRSPTSSAI | |
| PLQSPRNSGSFPSPSISPR | |
| B. Human MTUS1 Nucleic Acid (mRNA coding) Sequence | |
| (SEQ ID NO: 16) | |
| 1 | aaagggggcg gcagcgccgg cggagcggag gcgggtctca cgtgggccag cgcagagcct |
| 61 | gcggaaggga cggatgcgga tctcgtcgct gtcaccttga aagtgaccga ggggcttgac |
| 121 | tgtggactcc ttacgccgcc cacccgggcc cggcggtccc agccttctcg cagggcccct |
| 181 | tctcagcaga agcaagcggg gccgagaaag cgggtggaat agggttgctg caggtcccaa |
| 241 | agacccctcg tggcgcctcg ctactttctg cagcttgttt gcactttttc acgctctaga |
| 301 | aaaatctcat cttaattaag ggaacaacaa atcatttaat cttcagagca tcttagactg |
| 361 | aaaacctttc aactgtgctg aaaaacctag aagacagacc attttgccca ccctctcatt |
| 421 | taaaaggaat tgaagaagaa ataaaatggc agaggtttaa ggttactatt caggatgact |
| 481 | gatgataatt cagatgataa aatagaagat gaattgcaaa ccttctttac cagtgataaa |
| 541 | gatggaaata cacatgcata caacccgaaa tcaccaccta cacaaaactc ttcagccagc |
| 601 | agtgtgaact ggaattctgc caacccagat gacatggtgg ttgattatga aactgaccct |
| 661 | gctgtagtta ctggtgaaaa tatttcttta agccttcagg gtgttgaagt atttggtcat |
| 721 | gaaaagtctt ctagtgattt cattagtaag caggtgttag atatgcataa agattctatt |
| 781 | tgtcagtgtc ctgcacttgt aggtactgag aagcccaaat atctgcaaca cagttgtcat |
| 841 | tccctagaag cagttgaggg ccagagtgtt gagccatctt tgccttttgt gtggaagcct |
| 901 | aatgacaatt tgaactgtgc aggctactgt gatgccttgg agctaaacca aacatttgac |
| 961 | atgacagtgg ataaagttaa ctgcaccttt atatcacatc atgccatcgg aaagagtcag |
| 1021 | tccttccata ctgctggaag cctgccacca actggtagga gaagtggaag tacatcttct |
| 1081 | ttatcctatt ccacttggac atcttcccat tctgataaga cgcatgcaag agaaactact |
| 1141 | tatgatagag aaagctttga aaaccctcaa gtcacaccat cagaagccca agacatgact |
| 1201 | tacacagcat tttctgatgt ggtgatgcaa agtgaggttt ttgtttcaga tattggaaat |
| 1261 | cagtgtgcat gttcttcagg aaaggtcacc agtgagtaca cagatggatc acaacaaaga |
| 1321 | ctagttggag aaaaggagac acaagcacta acaccagttt ctgatggcat ggaagtcccc |
| 1381 | aatgattctg cattacaaga gttcttttgt ttatcccatg atgaatccaa tagcgaacca |
| 1441 | cattcacaga gctcatacag gcacaaggaa atgggccaaa atctgagaga gacagtgtcc |
| 1501 | tattgtctta ttgatgatga atgcccttta atggtgccag cttttgataa gagcgaagct |
| 1561 | caagtgctga acccagagca taaagtcact gagactgaag acacacaaat ggtctccaaa |
| 1621 | ggaaaggatt tgggaaccca aaatcatacc tcagaattga ttctaagtag cccgccagga |
| 1681 | caaaaggtgg gctcgtcatt tggactgact tgggatgcaa atgatatggt cattagcaca |
| 1741 | gacaaaacga tgtgcatgtc aacaccagtc ctagaaccca caaaagtaac cttttctgtt |
| 1801 | tcaccgattg aagcgacgga gaaatgtaag aaagtggaga agggtaatcg agggcttaaa |
| 1861 | aacataccag actcgaagga ggcacctgtg aacctgtgta aacccagttt aggaaaatca |
| 1921 | acaatcaaaa cgaatacccc aataggctgc aaagttagaa aaactgaaat tataagttac |
| 1981 | ccaagaccaa acttcaagaa tgtcaaagca aaagttatgt ctagagcagt gttgcagccc |
| 2041 | aaagatgctg ctttatcaaa ggtcacgccc agacctcagc agaccagtgc ctcatcaccc |
| 2101 | tcatcagtga attcaagaca acaaacagtc ttgagcagaa caccgagatc tgacttgaat |
| 2161 | gcagacaaaa aagcagaaat tctaattaac aagacacata agcagcagtt taataaactc |
| 2221 | attactagcc aggctgtgca tgttacaact cattctaaaa atgcttcaca cagggttcca |
| 2281 | agaacaacat ctgccgtgaa atcgaatcag gaagatgttg acaaagccag ttcttctaac |
| 2341 | tcagcatgcg agaccgggtc cgtttctgcg ttgtttcaga agatcaaagg catactccct |
| 2401 | gttaaaatgg aaagtgcaga atgtttggaa atgacctatg ttcccaacat tgataggatt |
| 2461 | agccctgaaa agaagggtga aaaagaaaat gggacatcta tggaaaaaca agagctgaaa |
| 2521 | caagagatta tgaatgagac ttttgaatat ggttctctgt ttttgggctc tgcttcaaaa |
| 2581 | acaacgacca cctcaggtag gaatatatcc aagcctgact cctgcggttt gaggcaaata |
| 2641 | gctgctccaa aagccaaagt ggggccccct gtttcctgtt tgaggcggaa cagtgacaat |
| 2701 | agaaatccca gtgctgatcg agccgtatct cctcagagga tcaggcgtgt gtccagttct |
| 2761 | ggaaagccta catccttgaa aactgcacag tcgtcatggg tgaatttgcc tagaccactt |
| 2821 | cctaaatcca aagcatcttt gaaaagtcct gcgctgcgga ggacaggaag caccccctca |
| 2881 | atagccagca cccacagtga gctgagcact tacagcaaca attctggtaa tgccgctgtc |
| 2941 | atcaaatatg aggagaaacc tccaaaacca gcatttcaga atggttcctc aggatccttt |
| 3001 | tatttgaagc ctttggtatc cagggctcat gttcacttga tgaaaactcc tccaaaaggt |
| 3061 | ccttcgagaa aaaatttatt tacagctctt aatgcagttg aaaagagcag gcaaaagaat |
| 3121 | cctcgaagct tatgtatcca gccacagaca gctcccgatg cgctgccccc tgagaaaaca |
| 3181 | cttgaattga cgcaatataa aacaaaatgt gaaaaccaaa gtggatttat cctgcagctc |
| 3241 | aagcagcttc ttgcctgtgg taataccaag tttgaggcat tgacagttgt gattcagcac |
| 3301 | ctgctgtctg agcgggagga agcactgaaa caacacaaaa ccctatctca agaacttgtt |
| 3361 | aacctccggg gagagctagt cactgcttca accacctgtg agaaattaga aaaagccagg |
| 3421 | aatgagttac aaacagtgta tgaagcattc gtccagcagc accaggctga aaaaacagaa |
| 3481 | cgagagaatc ggcttaaaga gttttacacc agggagtatg aaaagcttcg ggacacttac |
| 3541 | attgaagaag cagagaagta caaaatgcaa ttgcaagagc agtttgacaa cttaaatgct |
| 3601 | gcgcatgaaa cctctaagtt ggaaattgaa gctagccact cagagaaact tgaattgcta |
| 3661 | aagaaggcct atgaagcctc cctttcagaa attaagaaag gccatgaaat agaaaagaaa |
| 3721 | tcgcttgaag atttactttc tgagaagcag gaatcgctag agaagcaaat caatgatctg |
| 3781 | aagagtgaaa atgatgcttt aaatgaaaaa ttgaaatcag aagaacaaaa aagaagagca |
| 3841 | agagaaaaag caaatttgaa aaatcctcag atcatgtatc tagaacagga gttagaaagc |
| 3901 | ctgaaagctg tgttagagat caagaatgag aaactgcatc aacaggacat caagttaatg |
| 3961 | aaaatggaga aactggtgga caacaacaca gcattggttg acaaattgaa gcgtttccag |
| 4021 | caggagaatg aagaattgaa agctcggatg gacaagcaca tggcaatctc aaggcagctt |
| 4081 | tccacggagc aggctgttct gcaagagtcg ctggagaagg agtcgaaagt caacaagcga |
| 4141 | ctctctatgg aaaacgagga gcttctgtgg aaactgcaca atggggacct gtgtagcccc |
| 4201 | aagagatccc ccacatcctc cgccatccct ttgcagtcac caaggaattc gggctccttc |
| 4261 | cctagcccca gcatttcacc cagatgacac ctccccaaag tccacagact ctctgaaagc |
| 4321 | attttgatgc aggtctgcag gactgacccc aaggaggaac gtgggcacaa gaggtatatc |
| 4381 | agcacacgtg tgatcaccgt agggtaactg gagcgtcacc accggcggaa tcgcagcttc |
| 4441 | tgagactgga actctggagg aagacttttg cctccgtcca aaagattcct ccaaaaaaag |
| 4501 | atttaaaaaa agatttcggc atcgacacgg acgttgttgc acaaagcact taaagaacga |
| 4561 | gagcatcttg ttcattgcct ttttcaccta agcatagggg gaaaaactct cagggcccta |
| 4621 | ttaagattta taacctttgt aatgttcttc accacagaca ccttcttgtg agttttcagt |
| 4681 | ctgactgtgg gggtgggggg tgtgaatgaa atggatgtca cagagtgtca tgtgtctgat |
| 4741 | gcagcctcct ctgctgtgta ttaaatgtca aaatctgaat atatctggat atgtactaat |
| 4801 | caaataataa tcaatcaatc agcatataca tttcagccaa agccatagaa gaaaaagcaa |
| 4861 | tagttgcttg aattatgatc atctaccacc aactctgctc agccctgtaa cagggtaggg |
| 4921 | agagggtata acaggaagag ctttgacttg tccctgtcta tacattctct gtatcttttg |
| 4981 | ggggtaactt cttggcagtt tttcagtgtt cagccatgtc agttgaaact agatttttct |
| 5041 | gtagattttt tacttaccca tgtgagccta acactatcct gtaattcatt ttctcaggct |
| 5101 | atgtgtaaat gtagaaccct aatttttcta taaaaaaaca aactaactaa ctaactgtgt |
| 5161 | aaagaaagaa aaagggaagt accaatgggt ttttccacct tatttttacc tttgatctac |
| 5221 | ccttgcagat ttaacctgtc ttcttccctc ccattattct cattttcctt ttacctttct |
| 5281 | ccaccatcca gagccacaaa agcaaacctt ctacctccta cctacttttc tctgggacaa |
| 5341 | ggataaagga atatgatttt ccagagcccc agagccagct catcttccag gtgctgaaac |
| 5401 | cactttccaa ataaactaaa gcctggattt gatattacaa attttgggaa atcttagaat |
| 5461 | aaagaacgag aacaaggaag tcattggcta gtataattaa gaaaggtagg attcagtgct |
| 5521 | taccgatgat gcagtacttg atagaagaaa acagtctggg aggatagcgc tcatttttca |
| 5581 | gttacccttt aaggagtccc tttgtctttg ggaaagtagc agaatggtcc gcttctttcc |
| 5641 | catgagtgga aaatgtggct tgtccaactc tcctccaggt tgcatttcag tttctttcca |
| 5701 | aaacttatta cctcccctaa tcctgagact ttggaaaagg tggaaggaag aactgttgct |
| 5761 | ttatctcccc ctccctgcat gtgtcaacat tgtgatgtca gtatttacta atctacattc |
| 5821 | agtggctgta caaataacag ctgtagtaag aagagattca ggatgctaga ggtgaatatt |
| 5881 | tgggtcattt acatgtacac tacatagcaa gttgatactc atgttgcatg ttcttttaaa |
| 5941 | ttagtgattt tgtgtcttaa gtctttaact tccaatactt catcatgtat gtaaccttcc |
| 6001 | atgtttgctt ctgataaatg gaaatgtagg ttcactgcca cttcatgaga tatctctgct |
| 6061 | cacgcttcca agttgttctc aatgacatta gccaaagttg ggtttgccat tcatccccta |
| 6121 | ggcatggtaa atcttgtgtt gttccctgct gtcctccgta ttacgtgacc ggcaaataaa |
| 6181 | tctcatagca gttaatataa aacatctttg gaggatggga gagaacagga gggaagatgg |
| 6241 | gaaacaaaat agagaattct taagattttg tttaaaccaa atgtttcatg tagaatgcaa |
| 6301 | aatgttggca cgtcaaaaat atgaatgtgt agacaactgt agttgtgctc agtttgtagt |
| 6361 | gatgggaagt gtattttact ctgatcaaat aaataatgct ggaatactca agaattgcaa |
| 6421 | aaaaaaaaaa aaaaa |
| 9. NUP133 Gene | |
| A. Human NUP133 Polypeptide Sequence | |
| (SEQ ID NO: 17) | |
| MFPAAPSPRTPGTGSRRGPLAGLGPGSTPRTASRKGLPLGSAVSSPVLFSPVG | |
| RRSSLSSRGTPTRMFPHHSITESVNYDVKTFGSSLPVKVMEALTLAEVDDQLT | |
| INIDEGGWACLVCKEKLIIWKIALSPITKLSVCKELQLPPSDFHWSADLVALSY | |
| SSPSGEAHSTQAVAVMVATREGSIRYWPSLAGEDTYTEAFVDSGGDKTYSFL | |
| TAVQGGSFILSSSGSQLIRLIPESSGKIHQHILPQGQGMLSGIGRKVSSLFGILS | |
| PSSDLTLSSVLWDRERSSFYSLTSSNISKWELDDSSEKHAYSWDINRALKENI | |
| TDAIWGSESNYEAIKEGVNIRYLDLKQNCDGLVILAAAWHSADNPCLIYYSLI | |
| TIEDNGCQMSDAVTVEVTQYNPPFQSEDLILCQLTVPNFSNQTAYLYNESAVY | |
| VCSTGTGKFSLPQEKIVFNAQGDSVLGAGACGGVPIIFSRNSGLVSITSRENVS | |
| ILAEDLEGSLASSVAGPNSESMIFETTTKNETIAQEDKIKLLKAAFLQYCRKDL | |
| GHAQMVVDELFSSHSDLDSDSELDRAVTQISVDLMDDYPASDPRWAESVPEE | |
| APGFSNTSLIILHQLEDKMKAHSFLMDFIHQVGLFGRLGSFPVRGTPMATRLL | |
| LCEHAEKLSAAIVLKNHHSRLSDLVNTAILIALNKREYEIPSNLTPADVFFREV | |
| SQVDTICECLLEHEEQVLRDAPMDSIEWAEVVINVNNILKDMLQAASHYRQN | |
| RNSLYRREESLEKEPEYVPWTATSGPGGIRTVIIRQHEIVLKVAYPQADSNLR | |
| NIVTEQLVALIDCFLDGYVSQLKSVDKSSNRERYDNLEMEYLQKRSDLLSPLL | |
| SLGQYLWAASLAEKYCDFDILVQMCEQTDNQSRLQRYMTQFADQNFSDFLF | |
| RWYLEKGKRGKLLSQPISQHGQLANFLQAHEHLSWLHEINSQELEKAHATL | |
| LGLANMETRYFAKKKTLLGLSKLAALASDFSEDMLQEKIEEMAEQERFLLH | |
| QETLPEQLLAEKQLNLSAMPVLTAPQLIGLYICEENRRANEYDFKKALDLLEY | |
| IDEEEDININDLKLEILCKALQRDNWSSSDGKDDPIEVSKDSIFVKILQKLLKD | |
| GIQLSEYLPEVKDLLQADQLGSLKSNPYFEFVLKANYEYYVQGQI | |
| B. Human NUP133 Nucleic Acid (mRNA coding) Sequence | |
| (SEQ ID NO: 18) | |
| 1 | ctcttccctt aggtgtttaa gttccgcgcg caggccaggc tgcaacctga cggccagatc |
| 61 | cctcgctgtc ctagtcgctg ctccttggag tcatgttccc agccgcccct tctccgcgga |
| 121 | ccccgggtac cgggtcccga aggggcccgc tggccggact cgggcccggc tccacgcccc |
| 181 | ggacggctag caggaagggt ctgcccctgg ggtctgcagt cagctcccca gtgctcttct |
| 241 | cgccggtcgg ccggcgtagc tcgctaagct cgcggggaac accaacacga atgttcccac |
| 301 | accactccat aactgagtct gtgaactatg atgtgaaaac gtttggatct tctcttcctg |
| 361 | ttaaagtcat ggaagcccta acattggctg aagtcgatga ccagctgacc attaacatag |
| 421 | atgaaggtgg atgggcttgt ctggtgtgca aagagaagct cattatttgg aagattgctc |
| 481 | tgtcacctat tactaagtta tccgtttgca aagaacttca gctgccacct agtgatttcc |
| 541 | actggagtgc cgacttagtg gctctttctt actcttctcc ctcaggtgaa gcacattcta |
| 601 | ctcaggctgt tgctgtcatg gttgccacca gagaaggatc tatccgctat tggccaagcc |
| 661 | ttgctggtga agatacctac acagaggctt ttgtagattc gggaggtgat aagacttaca |
| 721 | gtttcctaac agcagtgcag ggaggaagtt ttattttgtc ttcatcagga agccaactaa |
| 781 | ttcggttgat acctgagagc tcaggaaaga ttcatcagga tatcctgcct caggggcaag |
| 841 | gcatgctttc aggaattggt cgaaaagttt cttctctttt tggaatttta tctcctagta |
| 901 | gtgatctcac actttcaagt gttctctggg atagagagag atcaagcttt tatagcctga |
| 961 | cgagttcaaa catcagtaaa tgggaattag atgattcttc agaaaagcat gcatacagtt |
| 1021 | gggatataaa tagagccctg aaggaaaaca ttaccgatgc tatttgggga tctgaaagta |
| 1081 | actatgaagc tattaaagaa ggagtcaaca ttcgatattt ggacttgaag caaaactgtg |
| 1141 | atgggctggt gattttggca gcagcatggc actcagcaga caatccatgt ctcatctatt |
| 1201 | actctctgat aacaatagaa gataatggtt gccaaatgtc agatgcagtt actgtagaag |
| 1261 | tcactcaata taatccacct tttcagtctg aagacctgat tttgtgtcag ttgacggtcc |
| 1321 | caaacttttc aaaccagact gcctatctgt ataacgaaag tgctgtctat gtgtgctcca |
| 1381 | caggaactgg gaaattttct cttccccagg agaaaattgt ctttaatgca caaggagata |
| 1441 | gtgttttagg tgctggtgcc tgtggtggtg ttcctatcat tttttctaga aacagtggac |
| 1501 | tggtgtctat tacttcaagg gaaaatgtgt ctatattggc agaagacttg gaagggtctt |
| 1561 | tagcatcttc agttgctgga ccaaacagtg agagtatgat ttttgagacc actacaaaga |
| 1621 | atgaaactat agcccaggaa gataaaatca agttgctgaa agctgccttt ctgcaatact |
| 1681 | gcagaaaaga tttaggtcat gctcaaatgg tggttgatga gctcttttcc tctcactctg |
| 1741 | atttggattc tgattctgaa ctagacaggg cagttaccca aatcagtgta gacctgatgg |
| 1801 | atgactaccc agcatctgac ccacggtggg ctgagtctgt ccctgaggaa gcacctgggt |
| 1861 | tcagcaatac gtcactgatt atccttcacc agctagaaga caagatgaaa gctcactctt |
| 1921 | ttcttatgga ctttattcat caagttggct tatttggacg tctaggcagt tttccagtta |
| 1981 | gagggacacc gatggccact cgactgttgc tctgtgagca tgccgaaaag ctgtcagccg |
| 2041 | ccattgttct caagaaccac cactcccggc tttctgacct tgtcaacaca gccatattga |
| 2101 | ttgctttgaa caagagggag tatgaaatcc catccaacct gactcctgca gatgtctttt |
| 2161 | tcagggaggt atcccaagta gataccatct gtgagtgctt actggagcat gaggagcaag |
| 2221 | tcttgaggga tgcacctatg gattccattg aatgggctga agtggtgatc aatgtgaaca |
| 2281 | atattctcaa ggatatgctg caggctgcta gtcattatcg ccaaaataga aactctttgt |
| 2341 | atagaagaga agaatcacta gaaaaagaac ctgaatatgt tccatggacg gcaacaagtg |
| 2401 | gtcctggtgg catccgaacg gtaataatac gccagcatga gattgtcctg aaggtggctt |
| 2461 | atccacaggc agacagcaac ctccgaaaca tcgtgaccga gcagctggta gccctgatcg |
| 2521 | attgcttcct ggatggttat gtttctcagc ttaagtctgt ggataaatcc agtaatcggg |
| 2581 | aaagatatga caatctggag atggaatacc tacagaaaag atcagatctc ttatctcctc |
| 2641 | ttctttcact aggccagtac ctgtgggctg cttctctagc agagaaatac tgtgactttg |
| 2701 | atatattggt acaaatgtgt gagcagactg acaaccagag ccgactccag cgctacatga |
| 2761 | cccagtttgc tgatcagaat ttttcagact ttctcttccg ttggtatctg gagaaaggaa |
| 2821 | agcgaggcaa attattatct cagcccattt ctcagcatgg acagttggca aattttttgc |
| 2881 | aagctcatga acatctcagc tggttacatg aaattaatag ccaagaatta gaaaaggctc |
| 2941 | atgcaacact tctgggtttg gcaaatatgg aaactcgtta ctttgcaaag aagaaaaccc |
| 3001 | ttcttggctt gagtaaattg gctgcattag cttcagactt ttcagaggat atgctacaag |
| 3061 | aaaaaattga agaaatggct gagaaggatc gctttctact gcatcaggag accctacctg |
| 3121 | aacagctgct ggcggagaaa cagctaaatc tcagtgcgat gccagtattg actgcaccac |
| 3181 | aactcattgg tctatatatc tgtgaagaaa atagaagagc taatgaatat gatttcaaga |
| 3241 | aagctttgga cttgttggaa tatattgatg aggaagaaga tataaatata aatgatctaa |
| 3301 | aactggaaat cctttgcaaa gctcttcaga gagataactg gtccagttct gatggcaaag |
| 3361 | atgatccaat tgaagtatct aaagacagta tatttgtgaa gatcttacag aaacttttaa |
| 3421 | aagatggcat tcagctcagt gagtacttac cggaggtgaa agacctgcta caagcggatc |
| 3481 | agcttggaag cttaaagtcc aatccttact tcgagtttgt tttgaaagca aattatgaat |
| 3541 | attatgttca gggacaaata taactttttc taaaaatggc cattgtttat gaaatctgta |
| 3601 | taagtgtgtc cttatacaaa ttttaggcca taaacaagtg taagtttgta caatttcata |
| 3661 | acatgtatag ctgagttttt atactttata tgtaggaagc taatataaaa tagttatgta |
| 3721 | actgtgattt tggttttcag ttatgtgact tgttttttcc acctgaaatg tgtcagttgt |
| 3781 | tgttcctgta ctcggtgccc tttcttttta ctctcacgtg gtcccaggtt ctggagttct |
| 3841 | tgtcctggtt ctagctgctc acatgtacaa atcacttcta ggcctcagtt tctgcgacta |
| 3901 | tgaaaattac tagattgcac tagcttgtct ctaaaattgc tgtgactcca gatactttgc |
| 3961 | actgaagaga atctagggtg tttgatatct gtttcagtta gggctaatgg gaaatgtcta |
| 4021 | gtaagataaa tgtcaacttt tgctgactta ttatgagatg aaaaaccaaa ggagagtggg |
| 4081 | cctaactcat gtgagcttga taactgatga actcattggg agcattttaa acttttctac |
| 4141 | ataaataata aatgagcact aatgaaagta |
| 10. ZNF93 Gene | |
| A. Human ZNF93 Polypeptide Sequence | |
| (SEQ ID NO: 19) | |
| MGPLQFRDVAIEFSLEEWHCLDTAQRNLYRNVMLENYSNLVFLGIVVSKPDL | |
| IAHLEQGKKPLTMKRHEMVANPSVICSHFAQDLWPEQNIKDSFQKVILRRYE | |
| KRGHGNLQLIKRCESVDECKVHTGGYNGLNQCSTTTQSKVFQCDKYGKVFH | |
| KFSNSNRHNIRHTEKKPFKCIECGKAFNQFSTLITHKKIHTGEKPYICEECGK | |
| AFKYSSALNTHKRIHTGEKPYKCDKCDKAFIASSTLSKHEIIHTGKKPYKCEE | |
| CGKAFNQSSTLTKHKKIHTGEKPYKCEECGKAFNQSSTLTKHKKIHTGEKPY | |
| VCEECGKAFKYSRILTTHKRIHTGEKPYKCNKCGKAFIASSTLSRHEFIHMGK | |
| KHYKCEECGKAFIWSSVLTRHKRVHTGEKPYKCEECGKAFKYSSTLSSHKRS | |
| HTGEKPYKCEECGKAFVASSTLSKHEIIHTGKKPYKCEECGKAFNQSSSLTK | |
| HKKIHTGEKPYKCEECGKAFNQSSSLTKHKKIHTGEKPYKCEECGKAFNQSS | |
| TLIKHKKIHTREKPYKCEECGKAFHLSTHLTTHKILHTGEKPYRCRECGKAF | |
| NHSATLSSHKKIHSGEKPYECDKCGKAFISPSSLSRHEIIHTGEKP | |
| B. Human ZNF93 Nucleic Acid (mRNA coding) Sequence | |
| (SEQ ID NO: 20) | |
| 1 | agacaccagg acccctggaa gcctagaaat gggaccattg caatttagag atgtggccat |
| 61 | agaattctct ctggaggagt ggcattgcct ggacactgca cagcggaatc tatataggaa |
| 121 | tgtgatgtta gagaactaca gtaacctggt cttccttggt attgttgtct ctaagccaga |
| 181 | cctgatcgcc catctggagc aaggaaaaaa acctttgact atgaagagac atgagatggt |
| 241 | agccaacccc tcagttatat gttctcattt tgcccaagat ctttggccag agcagaacat |
| 301 | aaaagattct ttccaaaaag tgatactgag aagatatgaa aaacgtggac atggaaattt |
| 361 | acagttaata aaaaggtgtg aaagtgtaga tgagtgtaag gtgcacacag gaggttataa |
| 421 | tggacttaac cagtgtagta caactaccca gagcaaagta tttcaatgtg ataaatatgg |
| 481 | gaaagtcttt cataaatttt caaattcaaa tagacataat ataagacata ctgaaaaaaa |
| 541 | acctttcaaa tgcatagaat gtggcaaagc ttttaaccag ttctcaaccc ttataacaca |
| 601 | taagaaaatt catactggag agaaacccta catttgtgaa gaatgtggca aagcctttaa |
| 661 | gtactcctct gcccttaata cacataagag aattcatact ggagagaaac catacaagtg |
| 721 | tgataaatgt gacaaagcct ttattgcatc ctcaaccctt agtaaacatg agatcattca |
| 781 | tactggaaag aaaccctaca agtgtgaaga atgtggcaaa gcttttaacc aatcctcgac |
| 841 | acttactaaa cataagaaaa ttcatactgg agagaaaccc tacaaatgtg aagaatgtgg |
| 901 | caaagctttt aaccaatcct caacacttac taaacataag aaaattcata ctggagagaa |
| 961 | gccctacgtt tgtgaagaat gtggcaaagc ctttaagtac tcccgtatcc ttactacaca |
| 1021 | taagagaatt catactggag agaaaccata caagtgtaat aaatgtggca aagcctttat |
| 1081 | tgcatcctca acccttagta gacatgagtt cattcatatg ggaaagaaac attacaaatg |
| 1141 | tgaagaatgt ggcaaagcct tcatttggtc ctcagtccta actagacata agagagttca |
| 1201 | tactggagag aagccctaca aatgtgaaga atgtggcaaa gcctttaagt actcctctac |
| 1261 | ccttagttca cataagagaa gtcatactgg agagaaaccc tacaaatgtg aagaatgtgg |
| 1321 | caaagctttt gttgcatcct caacccttag taaacatgag atcattcata ctggaaagaa |
| 1381 | accctacaag tgtgaagaat gtggcaaagc ttttaaccag tcctcatccc ttactaaaca |
| 1441 | taagaaaatt catactggag agaaacccta caaatgtgaa gaatgtggca aagcttttaa |
| 1501 | ccagtcctct tcccttacta aacataagaa aattcatact ggagagaaac cctacaaatg |
| 1561 | tgaagaatgt ggcaaagctt ttaaccagtc ctcaaccctt attaaacata agaaaattca |
| 1621 | tactagagag aaaccctaca aatgtgaaga atgtggcaaa gcttttcacc tatccacaca |
| 1681 | ccttactaca cataagatac ttcatactgg agagaaacct tatagatgta gagaatgtgg |
| 1741 | caaagctttt aaccattctg caaccctttc ttcacataag aaaatccatt ctggagagaa |
| 1801 | accatacgag tgtgataaat gtggcaaagc ctttatttca ccctcaagcc ttagtagaca |
| 1861 | tgagataatt catactgggg agaaacccta gaagtgtgaa gaatgtggca aagccttcaa |
| 1921 | gtggtcctca caccttacta tacactgaga gttctgaact tactctgtaa ccatcccaaa |
| 1981 | ctcctcccag |
| 11. RHBDL2 Gene | |
| A. Human RHBDL2 Polypeptide Sequence | |
| (SEQ ID NO: 21) | |
| MAAVHDLEMESMNLNMGREMKEELEEEEKMREDGGGKDRAKSKKVHRIV | |
| SKWMLPEKSRGTYLERANCFPPPVFIISISLAELAVFIYYAVWKPQKQWITLD | |
| TGILESPFIYSPEKREEAWRFISYMLVHAGVQHILGNLCMQLVLGIPLEMVHK | |
| GLRVGLVYLAGVIAGSLASSIFDPLRYLVGASGGVYALMGGYFMNVLVNFQE | |
| MIPAFGIFRLLIIILIIVLDMGFALYRRFFVPEDGSPVSFAAHIAGGFAGMSIGY | |
| TVFSCFDKALLKDPRFWIAIAAYLACVLFAVFFNIFLSPAN | |
| B. Human RHBDL2 Nucleic Acid (mRNA coding) Sequence | |
| (SEQ ID NO: 22) | |
| 1 | atggctgctg ttcatgatct ggagatggag agcatgaatc tgaatatggg gagagagatg |
| 61 | aaagaagagc tggaggaaga ggagaaaatg agagaggatg ggggaggtaa agatcgggcc |
| 121 | aagagtaaaa aggtccacag gattgtctca aaatggatgc tgcccgaaaa gtcccgagga |
| 181 | acatacttgg agagagctaa ctgcttcccg cctcccgtgt tcatcatctc catcagcctg |
| 241 | gccgagctgg cagtgtttat ttactatgct gtgtggaagc ctcagaaaca gtggatcacg |
| 301 | ttggacacag gcatcttgga gagtcccttt atctacagtc ctgagaagag ggaggaagcc |
| 361 | tggaggttta tctcatacat gctggtacat gctggagttc agcacatctt ggggaatctt |
| 421 | tgtatgcagc ttgttttggg tattcccttg gaaatggtcc acaaaggcct ccgtgtgggg |
| 481 | ctggtgtacc tggcaggagt gattgcaggg tcccttgcca gctccatctt tgacccactc |
| 541 | agatatcttg tgggagcttc aggaggagtc tatgctctga tgggaggcta ttttatgaat |
| 601 | gttctggtga attttcaaga aatgattcct gcctttggaa ttttcagact gctgatcatc |
| 661 | atcctgataa ttgtgttgga catgggattt gctctctata gaaggttctt tgttcctgaa |
| 721 | gatgggtctc cggtgtcttt tgcagctcac attgcaggtg gatttgctgg aatgtccatt |
| 781 | ggctacacgg tgtttagctg ctttgataaa gcactgctga aagatccaag gttttggata |
| 841 | gcaattgctg catatttagc ttgtgtctta tttgctgtgt ttttcaacat tttcctatct |
| 901 | ccagcaaact ga |
| 12. DNAJC15 Gene | |
| A. Human DNAJC15 Polypeptide Sequence | |
| (SEQ ID NO: 23) | |
| MAARGVIAPVGESLRYAEYLQPSAKRPDADVDQQRLVRSLIAVGLGVAALAFA | |
| GRYAFRIWKPLEQVITETAKKISTPSFSSYYKGGFEQKMSRREAGLILGVSPSA | |
| GKAKIRTAHRRVMILNHPDKGGSPYVAAKINEAKDLLETTTKH | |
| B. Human DNAJC15 Nucleic Acid (mRNA) Sequence | |
| (SEQ ID NO: 24) | |
| 1 | agtctccggg ccgccttgcc atggctgccc gtggtgtcat cgctccagtt ggcgagagtt |
| 61 | tgcgctacgc tgagtacttg cagccctcgg ccaaacggcc agacgccgac gtcgaccagc |
| 121 | agagactggt aagaagtttg atagctgtag gcctgggtgt tgcagctctt gcatttgcag |
| 181 | gtcgctacgc atttcggatc tggaaacctc tagaacaagt tatcacagaa actgcaaaga |
| 241 | agatttcaac tcctagcttt tcatcctact ataaaggagg atttgaacag aaaatgagta |
| 301 | ggcgagaagc tggtcttatt ttaggtgtaa gcccatctgc tggcaaggct aagattagaa |
| 361 | cagctcatag gagagtcatg attttgaatc acccagataa aggtggatct ccttacgtag |
| 421 | cagccaaaat aaatgaagca aaagacttgc tagaaacaac caccaaacat tgatgcttaa |
| 481 | ggaccacact gaaggaaaaa aaaagagggg acttcaaaaa aaaaaaaaaa gccctgcaaa |
| 541 | atattctaaa acatggtctt cttaattttc tatatggatt gaccacagtc ttatcttcca |
| 601 | ccattaagct gtataacaat aaaatgttaa tagtcttgct ttttattatc ttttaaagat |
| 661 | ctccttaaat tctataactg atcttttttc ttattttgtt tgtgacattc atacattttt |
| 721 | aagatttttg ttatgttctg aattcccccc tacacacaca cacacacaca cacacacaca |
| 781 | cgtgcaaaaa atatgatcaa gaatgcaatt gggatttgtg agcaatgagt agacctctta |
| 841 | ttgtttatat ttgtaccctc attgtcaatt tttttttagg gaatttggga ctctgcctat |
| 901 | ataaggtgtt ttaaatgtct tgagaacaag cactggctga tacctcttgg agatatgatc |
| 961 | tgaaatgtaa tggaatttat taaatggtgt ttagtaaagt aggggttaag gacttgttaa |
| 1021 | agaaccccac tatctctgag accctatagc caaagcatga ggacttggag agctactaaa |
| 1081 | atgattcagg tttacaaaat gagccctgtg aggaaaggtt gagagaagtc tgaggagttt |
| 1141 | gtatttaatt atagtcttcc agtactgtat attcattcat tactcattct acaaatattt |
| 1201 | attgacccct tttgatgtgc aaggcactat cgtgcgtccc ctgagagttg caagtatgaa |
| 1261 | gcagtcatgg atcatgaacc aaaggaactt atatgtagag gaaggataaa tcacaaatag |
| 1321 | tgaatactgt tagatacaga tgatatattt taaaagttca aaggaagaaa agaatgtgtt |
| 1381 | aaacactgca tgagaggagg aataagtggc atagagctag gctttagaaa agaaaaatat |
| 1441 | tccgatacca tatgattggt gaggtaagtg ttattctgag atgagaatta gcagaaatag |
| 1501 | atatatcaat cggagtgatt agagtgcagg gtttctggaa agcaaggttt ggacagagtg |
| 1561 | gtcatcaaag gccagccctg tgacttacac tgcattaaat taatttctta gaacatagtc |
| 1621 | cctgatcatt atcactttac tattccaaag gtgagagaac agattcagat agagtgccag |
| 1681 | cattgtttcc cagtattcct ttacaaatct tgggttcatt ccaggtaaac tgaactactg |
| 1741 | cattgtttct atcttaaaat actttttaga tatcctagat gcatctttca acttctaaca |
| 1801 | ttctgtagtt taggagttct caaccttggc attattgaca tgttaggcca aataattttt |
| 1861 | tttgtgggag gtctcttgtg cgttttagat gattagcaat aatccctgac ctgttatcta |
| 1921 | ctaaagacta gtcgtttctc atcagttgtg acaacaaaaa tggttccaga tattgccaaa |
| 1981 | tgccctttag aggacagtaa tcgcccccag ttgagaacca tttcagtaaa actttaatta |
| 2041 | ctattttttc ttttggttta taaaataatg atcctgaatt aaattgatgg aaccttgaag |
| 2101 | tcgataaaat atatttcttg ctttaaagtc cccatacgtg tcctactaat tttctcatgc |
| 2161 | tttagtgttt tcacttttct cctgttatcc ttgtacctaa gaatgccatc ccaatcccca |
| 2221 | gatgtccacc tgcccaaagt ctaggcatag ctgaaggcca agctaaaatg tatccctctt |
| 2281 | tttctggtac atgcagcaaa agtaatatga attatcagct ttctgagagc aggcattgta |
| 2341 | tctgtcttgt ttggtgttac attggcaccc aataaatatt tgttgagcga aaaaaaaaaa |
| 2401 | aaaa |

1. A non-invasive method of identifying oocytes that are capable of giving rise to a viable pregnancy when fertilized comprising the following steps:
(i) obtaining at least one cumulus cell associated with an oocyte that is to be tested for pregnancy competency from a female donor or for other oocytes of said same donor;
(ii) assaying the expression of at least one gene by said at least one cumulus cell, the expression of which correlates to the capability of an oocyte associated with said cell to yield a viable pregnancy upon fertilization and transferal into a suitable uterine environment wherein said genes are selected from FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1), or their orthologs, splice or allelic variants or any combination of 1, 2, 3, 4, 5, 6, 7, 8, 9, 10, 11 or 12 of said genes; and
(iii) identifying, based on the level of expression of said at least one gene as compared to the characteristic level of expression by a cumulus cell associated with a pregnancy competent oocyte whether said oocytes or another oocyte derived from said female donor is potentially capable of yielding a viable pregnancy upon fertilization and transferal into a suitable uterine environment.

2-13. (canceled)

14. The method of claim 1, wherein:
(i) said oocyte and cumulus cell is mammalian.
(ii) said oocyte and cumulus cell is human.
(iii) said oocyte and cumulus cell is from a non-human primate oocyte.
(iv) the method of assaying gene expression uses a method that monitors differential gene expression;
(v) the method comprises indexing differential display reverse transcriptase polymerase chain reaction (DDRT-PCR);
(vi) the oocyte is obtained from a human female who is at least 25 years old;
(vii) the oocyte is obtained from a human female who is at least 30 years old.
(viii) the oocyte is obtained from a human female who is at least 35 years old;
(viii) the oocyte is obtained from a human female who is at least 40 years old;
(ix) the aberrant expression of said at least one gene is correlated to a condition selected from menopause, cancer, ovarian dysfunction, ovarian cyst, autoimmune disorder and hormonal dysfunction; and/or
(x) or any combination of the foregoing.

15-23. (canceled)

24. A method of assessing the efficacy of a fertility treatment comprising:
(i) treating a human female with a putative fertility enhancing treatment;
(ii) obtaining an oocyte and cumulus cells associated therewith from said human female after treatment and measuring the expression of at least one gene selected from those contained in Table 4 and further including FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834 m1), ND NUP133 (Hs00217272_m1), or their orthologs, splice or allelic variants by at least one cumulus cell associated with said oocyte or any combination of 1, 2, 3, 4, 5, 6, 7, 8, 9, 10, 11 or 12 of said genes and
(iii) evaluating whether said treatment is effective based on the level of expression of said at least one gene by said oocyte-associated cell as compared to the characteristic level of expression of said gene by a cumulus cell associated with a normal or pregnancy oocyte or other appropriate control.

25-36. (canceled)

37. The method of claim 24, wherein:
(i) said fertility treatment comprises hormonal therapy;
(ii) the subject is menopausal and the treatment comprises hormone replacement therapy;
(iii) gene expression is detected by real-time polymerase chain reaction (RT-PCR).
(iv) gene expression is detected differentially by indexing differential display reverse transcriptase polymerase chain reaction (DDRT-PCR);
(v) gene expression results are obtained using RNA from a cumulus cell; or
(vi) any combination of the foregoing.

38-42. (canceled)

43. A method of evaluating fertility potential in a subject comprising detecting the expression levels of specific pregnancy signature genes selected from those in Table 4, Table 12 or selected from FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1), or their orthologs, splice or allelic variants and ABCA6, NCAM1, OLFML3, PTPRA, SDF4, GPR137B, DDIT4, DUSP1, GPR137B, IDUA, KCTD5, NDNL2, SLC26A3, and TERF21P, or their orthologs, splice or allelic variants, or any combination of 1, 2, 3, 4, 5, 6, 7, 8, 9, 10, 11 or 12 of said genes, by a cumulus cell associated with an oocyte whose pregnancy potential is being evaluated or another oocyte collected from said subject, comparing said levels of expression to the characteristic levels of expression of said genes by cumulus cells which are associated with an oocyte capable of yielding a viable pregnancy; and determining whether said subject is potentially “pregnancy competent” based on whether said cumulus cell expresses one or more pregnancy signature genes at levels characteristic of pregnancy competent oocytes.

44-53. (canceled)

54. The method of claim 1, for selecting a competent oocyte or a competent embryo, further comprising a step of measuring the expression level of one or more genes selected from ABCA6, NCAM1, OLFML3, PTPRA, SDF4, GPR137B, DDIT4, DUSP1, GPR137B, IDUA, KCTD5, NDNL2, SLC26A3, and TERF21P or their orthologs, splice or allelic variant or any combination thereof by said cumulus cell or cumulus cells from the same female donor.

55. The method of claim 24, further comprising a step of measuring the expression level of one or more genes selected from ABCA6, NCAM1, OLFML3, PTPRA, SDF4, GPR137B, DDIT4, DUSP1, GPR137B, IDUA, KCTD5, NDNL2, SLC26A3, and TERF21P or their orthologs, splice or allelic variant or any combination thereof by said cumulus cell or cumulus cells from the same female donor.

56. The method of claim 1, wherein comparison of gene expression of the at least gene by the cumulus cell and the control is performed using a method selected from the group consisting of: weighted voting, Bayesian compound covariate, diagonal linear discriminant, nearest centroid, k-nearest neighbors, shrunken centroids, support vector machines, compound covariate, and any combination thereof.

57. The method of claim 56, wherein comparison of gene expression of the at least one gene by a cumulus cell associated with an oocyte that is to be tested for pregnancy competency to the characteristic level of expression by a cumulus cell associated with a pregnancy competent oocyte is performed using weighted voting.

58. The method of claim 1, further comprising producing an indicator that indicates whether said oocytes derived from said female donor is potentially capable of yielding a viable pregnancy upon fertilization and transferal into a suitable uterine environment.

59. The method of claim 58, wherein said indicator is provided as a report.

60. The method of claim 58, wherein said indicator is displayed on an electronic display.

61. The method of claim 58, wherein said indicator is provided as an electronic communication.

62. An array or detection kit composition for use in claim 1, containing at least 2 of the following genes, polypeptides encoded thereby, probes that specifically bind to the polypeptide or nucleic acid expression product at least 2 of said genes, primers that result in the specific amplification of mRNAs that encode at least 2 of the expression product of these genes, or antibodies that specifically bind to at least 2 of the polypeptides encoded by said genes wherein said genes are selected from: FGF12, (Hs00374427_m1), GPR137B (Hs00162803_m1), SLC2A9 (Hs00417125_m1), ARID1B (Hs00368175_m1), NR2F6 (Hs00172870_m1), ZNF132 (Hs01036387_m1), FAM36A (Hs00831105_s1), ZNF93 (Hs01656246_s1), RHBDL2 (Hs00384848_m1), DNAJC15 (Hs00387763_m1), MTUS1 (Hs00826834_m1), ND NUP133 (Hs00217272_m1), or their orthologs, splice or allelic variants or any combination of 1, 2, 3, 4, 5, 6, 7, 8, 9, 10, 11 or 12 of said genes.

63-67. (canceled)

68. The one or more array or detection kits according to claim 62 that includes one or more detectable labels.

69. The array or detection kits according claim 62, that includes directions in how to use in assays for detecting the level of expression of at least 2 of said 12 genes by cumulus cells associated with a donor woman's oocyte relative to a control which comprises the level of expression of the same genes by cumulus cells which are associated with normal oocytes (oocytes that are capable of giving rise to viable pregnancy naturally or in an IVF procedure).

70-75. (canceled)
